(19) United States
(12) Patent Application Publication
(10) Pub. No.: US 2023/0329561 A1
(43) Pub. Date: Oct. 19, 2023

(54) System and Method for Personalized Telehealth Management with Dynamic Monitoring

(71) Applicant: Zhiqiang CUI, Elmhurs, NY (US)

(72) Inventor: Zhiqiang Cui, Elmhurs, NY (US)

(21) Appl. No.: 18/022,167

(22) PCT Filed: Aug. 26, 2021
(86) PCT No.: PCT/US2021/047768

§ 371 Date: Feb. 20, 2023

Related U.S. Application Data
(60) Provisional application No. 63/070,599, filed on Aug. 26, 2020.

(51) Int. Cl. A61B 5/01; A61B 5/00

(52) U.S. Cl. CPC A61B 5/015; A61B 5/4854; A61B 5/0008; A61B 5/0022; A61B 2562/0271; A61B 2560/0242

(57) ABSTRACT

The present invention relates generally to a novel personalized telehealth management system and a novel method to synchronously detecting the dynamics of the temperature difference of symmetrical points of the human body for management of human health. Using high-resolution temperature sensors, combined with dynamic data of symmetrical changes in physiological indicators such as blood pressure, pulse, blood oxygen, and blood sugar, and the surrounding environment and symptom information, the proposed method is capable of judging physical and functional balance values through artificial intelligence. This invention provides a new clinical dynamic monitoring method of Chinese and Western medicine, and enables an accurate personalized health management system CWPAS, which is the abbreviation of check, warning, prevention, assessment and overall solution. Simply using an individual's left and right symmetrical temperature difference, the method is able to quantify disease and health care status, and design a personalized treatment plan based on the direction and absolute value of the symmetrical temperature difference as well as other symmetrical life information difference including pulse and blood pressure. The proposed method can be used to precisely monitor a patient's health status and dynamically evaluate the patient's treatment plan. With the use of the Internet of Things and artificial intelligence analysis, the method can be continuously updated and upgraded, resulting in greater social and economic benefits.

### FIELD OF THE INVENTION

The invention relates generally to a CWPAS health management system using symmetrical temperature difference of human body physiological indicators (CWPAS is the abbreviation of check, warning, prevention, assessment and overall solution). The point is that a healthy human body should be functionally balanced and symmetrical in shape. The disease of the human body first appears functional imbalance, the severity of the imbalance and the severity of the disease is related, this stage of the disease is still in the scope of functional lesions, if the disease is further aggravated, the asymmetry of the shape is produced namely organic lesions. Whether functional or organic, symmetrical differences in body temperature are reflected in the body, which can be detected and used for diagnosis. It is helpful for the treatment and rehabilitation of diseases to adjust the temperature of symmetrical parts of human body correspondingly. This theory was the first to suggest a symmetrical balance of body temperature, The present invention is the first to propose the theory of symmetry balance of body temperature. This theory is the first to put forward the idea of symmetrical balance of body temperature, creatively using the temperature difference of symmetrical points of the human body, using high resolution temperature sensor, to judge the balance value of human body's physiological indicators and function. A health management and clinical dynamic monitoring system that guides clinical and precise personalized lifestyle habits. In the same individual, the primary contradiction of qualitative disease and health care is quantified by grasping the simple temperature difference and calculating its correlation with other symmetrical vital information and external factors such as symptom environment. With the help of IoT tools, the system can keep on timely upgrading, and produce great social and economic benefits.

### BACKGROUND OF THE INVENTION

The health industry has so far not produced an authoritative leader or a standardized development model, because various interventions based on differentiated diagnoses yield discrepancy of effects on individuals at varied times and spaces, that is, the uncertainty of curative effect.

At present, medicine is based on the “statistical average level” of most patients, that is, the standardized medical model. Health evaluation is based on physical examinations, adopting the exclusion concept of “health is no diseases”. Due to a longtime span, it is unable to monitor the relationship between part and the whole, and observe the dynamic changes only by observing the local conditions of certain organs and components at a certain time, let alone the intricate correlation between the internal and external environments.

In daily life, we make good use of “analogy” to adopt minor adjustments and learn from what others do. Analogy provides a shortcut, free of heavy thinking, and improves efficiency.

However, in the field of health, if someone or some place's success model is copied in analogy, there may be uncertainty or disarray on the curative effects. That is because human health is affected by differences in time, space, and individual physique. To promote one experience to broad practice of lifestyle and health model will definitely result in differences. That is the fundamental reason for the result discrepancy of statistical analysis of collected data on lifestyles and medical behaviors.

There are no two leaves that are exactly the same, nor two individuals. Time passes by and circumstances are changing all the time, which is the objective and theoretical basis for individualized lifestyle and health interventions. Yet popularization requires standardization. The coupling of standardization and individualization is the optimal individualization.

As differences are ubiquitous, how can standards be formulated? Precision medicine takes DNA nuances as the standard. It is scientific, is being perfected, and it is the future direction. There are also theoretical generalizations and syndrome differentiation from the perspective of blood types or 9 physiques, as the standard. However, these are either lack of objectivity, lack of numeric expression, or lack of comprehensive correspondence, and therefore cannot be implemented to date.

Physique is an ancient and young medical proposition, and is the basis of individualization. Wikipedia's definition of The natural physique, or physical structure, of a person (see somatotype, anthropometry, body shape) focuses on the structure, reflecting Western medical views. “Cihai” defines it as “the inherent characteristics that are functionally and morphologically stable expressed by people on the basis of heredity and acquisition.” In fact, it is the integration of DNA gene expression in different environments, coping with that DNA expression is the basis for individualization. However, genetic testing is yet immature for health prediction, and the cost is relatively high. In the meantime, new wave of artificial intelligence is sweeping the world and quickly integrated into medical care. Therefore, for more than ten years, we have started with body surface life information, integrating the 21st century pioneer physics' symmetry breaking and the core of precision medicine “symmetrical difference and personalization” into physical fitness, and building “the objective standard of physical fitness” through more than 10,000 person times clinical practices. (1) At present, hospitals only measure unilateral temperature, blood pressure, pulse, etc. for comparison, which is a one-dimensional measurement. Symmetrical balance measures symmetrical life information at the same time, and diagnoses physique, health and disease status based on the information differences, forming a three-dimensional thinking mode with symmetrical information, information differences, and time. (2) Clinical experiments have confirmed that the left axillary body temperature corresponds to the temperature of the upper body, and the right axillary body temperature corresponds to that of the lower body. The physique is categorized into two types: when left axillary body temperature is higher than the right, “upper hot and lower cool”, and vice versa. The trend of symmetry breaking is a reflection of the type of physique and the direction of disease development and rehabilitation.
(3) The direction and value of temperature difference is the “balance coefficient”, which is the direction and value of physical fitness, rehabilitation and lifestyle. Physical fitness and the degree of imbalance are represented by numbers. If a part of the body is extremely warm, it should be cooled down, and vice versa, to make the body balanced and healthy. According to the direction and value of the temperature difference, the corresponding lifestyle, exercise types, rehabilitation methods and psychological balance are adopted.
(4) Symmetry and balance equal health. Diseases first cause imbalance at the functional level, and changes in quantity and quality result in structural and morphological changes.
(5) Symmetrical balance is tested concurrently, and basically under the same conditions, vital information and symmetrical differences deviate from the normal range as to reflect differences in the molecular structure and function of bilateral tissues, cells, genes, proteins, etc., just like hospital CT, MRI and various laboratory tests, ultrasound and other examinations to monitor diseases. The examinations focus on the normal range, and as long as it is within this range, health is stable.
(6) In the past twelve years, we measured the body temperature of more than 100 volunteer participants in the United States and China for 10 consecutive days. After statistical and mathematical modeling, we found that none of the values were the same, just as no one had the same DNA sequence. Under basic conditions, the temperature difference of healthy people is below ±0.2° C., the blood pressure difference between the two sides is below 10 Hgmm, and the left and right pulses are the same (about 7-8% of the population); in the case that the temperature difference exceeds ±0.2° C., the blood pressure difference is more than 10 mmHg, and the pulse difference is one time, it indicates a sub-health condition; whereas when the temperature difference exceeds ±0.3-0.35° C., the pulse difference is 2 times, the blood pressure difference is more than 20 mmHg, that is the disease stage; when the pulse difference is 3 times or the bilateral temperature difference is more than ±0.45° C., it is the life state imbalance and indicative of disease in aggravation. The more serious the disease, the greater the difference in symmetrical life information such as temperature difference, while change in reduction of the temperature difference suggests disease in relief. The change reflects the functional change and the quantitative relationship. The imbalanced sub-health state measurement can be found before the tissue morphology changes (except the 1-5‰ group of strong physical fitness).
(7) With the changes in physiological and pathological conditions of symmetrical parts, different symmetrical information is generated in the body and on the body surface. Collecting symmetrical information is only a process. The key, however, is to extract accurate symmetrical information difference (corresponding to DNA sequence difference and clinical testing), and analysis of its correlation with the above indicators, which is the “Da Vinci Code” or “Life Code”.

Due to the objective existence of differences, it is fundamental to health maintenance by establishing

classification standards on the basis of different time, space, and physical conditions using analogy. Take the Mediterranean diet for example, it generally refers to the lifestyle diet of the Mediterranean coast mainly composed of vegetables, fruits, fish, whole grains, legumes, and olive oil. Studies have found that it can reduce the risk of heart disease, protect the brain from vascular damage, and lower the risk of stroke and memory loss. Local statistical analysis has great effects. However, some experiments have been done in the United States, and the treatment evaluation index NNT is extremely low. It is hard to believe: 30 heart disease survivors adhered to the Mediterranean diet for 4 years yet only turned out prevention of 1 death, 61 people adhered to the Mediterranean diet and only 1 person avoided sudden heart failure. We opine that both conclusions may be viable, while using it in different regions and people has produced different results. The key explanation is that there is no objective standard for personalized assessment. Therefore, it is impossible to distinguish how different regions (including natural environment and social environment), race, and lifestyle should adopt different intervention methods, causing observational confusion and statistical mismatch. Symmetrical balance medicine is a work that changes medical cognitive mode. Through dynamic collection of symmetrical life information, it performs overall omni-quantification of the human body's symmetrical structure, natural endowment, physical condition, functionality, psychological state, information, energy, lifestyle, environmental factors, and intervention methods: (1). “Establishing objective standards for physical fitness”: The physique is categorized into two types. When the left axillary temperature is higher than the right—“upper hot and lower cool”, and vice versa. The direction and value of body temperature difference point to the direction and value of physical fitness, rehabilitation, and lifestyle. (2). Establishing a personalized digital measurement of health status: collect data on the symmetrical spots of the human body and extract life information contained therein at the same time and under basically similar conditions. Such includes measuring the symmetrical pulse and blood pressure difference through both wrists; measuring the left and right body temperature under both armpits, and so forth. At the same time, also record the concurrent health status and surrounding environment (temperature, humidity, etc.) as data for the person under test under that condition. (3). All-round dynamic digitization; the time of data acquisition is grouped into before- and after-collecting these basic symmetrical life information, before and after exercise, or that of rehabilitation, before and after psychological activities, etc. The symmetrical life information collected at different times reflects different life conditions and clinical significance, and the directions and values of information difference increase or decrease are the objective standards for health intervention. (4). The change of symmetrical life information difference corresponding to the time, space and physique is an objective indicator of the health changes. (5). Temperature is a statistical indicator of molecular average kinetic energy; symmetrical body temperature difference reflects the difference in average kinetic energy of symmetrical organs, tissues, and cells, and is the standardization basis for molecular average kinetic energy. Body temperature difference is the basic data to quantify life activities. (6). In case of major changes or qualitative changes in physical condition or illness, the correlation or causality is reflected by the information difference. According to their different directions and values, you may accurately assess whether the lifestyle, environmental factors, psychological activities, and intervention methods are beneficial or harmful, and identify hidden health hazards or bad habits. (7). The biggest factor that environment affects human being is the effect of cold and heat; the influence of environmental factors can be reflected by temperature. (8). The basis of precision diagnosis and targeted intervention: there are four seasons in a year, and the climate is lukewarm, hot, cool, and cold; the temperature difference has a direction, and the physical body has cold and hot; exercise heat dissipation is a vector; food has “four energy features” of lukewarm, hot, cool, and cold; temperament differs by “passionate or indifferent”; various aspects converge in direction of “cold and hot”, and subsequently the mapping or orientation of health stands out, ultimately forming “a unified expression of physique/lifestyle/environmental information for one intervention method”. (9). Quantifying the direction of rehabilitation: physical conditions and the data difference before and after rehabilitation can be matched to determine the treatment direction and achieve the long-term maintenance of curative effect. (10). Acupuncture quantification may solve the problem of “variable and inconsistent” evidence for the effectiveness of acupuncture under any circumstances. (11). Find out the greatest common divisor of various medical diagnoses in the world—body temperature, so that the integration and complementarity of various medicine disciplines becomes possible. (12). All-round dynamic digitization can organically integrate evidence-based medicine and artificial intelligence. This is the only method in the world that can objectively quantify lifestyle, environment, psychological activities, and intervention methods, as well as sustain empirical testing. The health management system of the present invention starts from the perspective of symmetrical balance, defines and monitors physical conditions from the basic laws of physics, and implements individualization: space-time is the basic definition of physics. Based on observing space-time and environmental information, collecting dynamic difference information of imbalance on morphology and functionality, then such information is used as criterion to distinguishing physical conditions, exercise pattern, lifestyle, and curative effect. It combines both function and dynamic structure. The quantitative change from functional imbalance to structural imbalance causes the transformation between the two. There is both time vector and space frame. The numerical value of the difference between left and right body temperature has overcome the abstraction of the concept “balance” as well as the sense of segregation or isolation of the modern morphological medicine. The difference objectivity, relevance, temporal and spatial dynamics, personalization, digitization, and standardization are all reflected, so as to quantify the dynamics of human health, lifestyle, and rehabilitation.

Currently in the Internet era, more and more data are collected for health management, and wearable medical devices and systems continue to debut, with huge development potential. Yet most of them are at the stage of information collection, display, transmission, and product sales. They are is mostly inaccurate, poor in stability, severe in homogeneity in the collection of vital information such as body temperature, pulse, blood pressure, blood sugar, ECG, etc., lacking in pertinence data support for the final clinical effect. Data processing is far from effective in supporting clinical practice. The relevance of collected data is not consistent with the efficacy of intervention, and there is no obvious advantage in reducing mortality and extending life span. How can emerging technologies better serve medicine?

The current medical system is built on morphology, and only morphological changes caused by diseases can be detected. While in the stage of changes in health imbalance, there is a lack of objective standards for diagnosis and normative intervention measures. The configuration of medical devices is merely catered to the needs of morphological observation.

The accuracy level does not need to be too precise. If the body temperature measures up to ±0.5° C., the device can be good enough to use as a Class 2 medical instrument. However, in the example of the female temperature, when follicle ruptures after ovulation, the body temperature drops by 0.04° C. After the rupture, progesterone is secreted, stimulates the central body temperature hub, in turn the body temperature rises by 0.4-0.6° C. Existing thermometers are limited to detect this process. Many other examples in similar illustration go on and so forth.

At present, the standardized medical model is shifting to the personalized medical model, and static testing is transformed into dynamic observation: physical, time, space, and dynamic observation methods, yet the existing medical framework with its localized, isolated, static, and segmented observation method has not evolved to a holistic, dynamic, and interconnected observation method, therefore inadequate to adapting to this development speed. There is a lack of core technologies and objective indicators for quantification and observation of physical condition, lifestyle, and health interventions that can be paired with one another.

Natural medicine has attracted more and more attention, yet more on the cultural perspective. For example, traditional Chinese medicine (TCM) emphasizes to account for broadness and profoundness, has survived a history of several thousand years, and relates great significance from the environment. However, with the doctrine belief of harmony between man and nature and syndrome differentiation and treatment,

TCM seldom applies clinical symptom quantification therefore affecting the curative effects. Acupuncture, massage therapy, self-exercise, physical activity, moxibustion, and an array of healthcare products are all self-promoted as the No. 1 cure in the world, with some success case being cited as proof. However, reports of cure rates, probability of curative effects, and sustainability are rarely seen, let alone the application of personalized classification.

At present, people have paid more and more attention to health, and timing for early detection of diseases is advanced greatly. People have become more aware of the importance of lifestyle toward health. However, different opinions emerge abundantly on how to obtain a scientific lifestyle, and some of them may even contradict to see people bewildered. Why is this happening? It's because only the importance of lifestyle is emphasized, and there is a lack of the principles of individualized treatment as well as the standards of individualized lifestyles.

The challenging issues of remote health management and the reasons why it cannot progress rapidly: (1) It is only used for the transmission of information and images. (2) It is limited to the collection and partial analysis of life information of standardized medical models. (3) It only focuses on the compliance of standardized treatment guidelines. 4) It only values the big data of life information collection, but fails to grasp the qualitative change data such as the transfer from medical treatment to health care and vice versa. 5) Disregarding the working effects of clinical application, all the documentation is copied blindly.

### BRIEF SUMMARY OF THE INVENTION

The present invention proposes the theory of symmetrical balance of body temperature for the first time and discloses a method for remote synchronous detection of dynamic characteristics of temperature difference of symmetrical points of human body. A high-resolution temperature sensor is adopted, combined with the dynamic data of symmetrical changes in physiological indicators such as blood pressure, pulse, blood oxygen and blood glucose, and the information of surrounding environment and symptoms is recorded at the same time, and the balance value of physique and function is judged by artificial intelligence. Add traditional Chinese and western medicine clinical dynamic monitoring means. Precise personalized life habits CWPAS health management system (CWPAS is abbreviation of Check, Warning, Prevention, Assessment and Solution). The simple symmetrical temperature difference between the right and the left of the same individual can be used to quickly assess the health status of the subjects and to identify the main contradictions between diseases and health care. The Internet of things is constantly updated and upgraded, resulting in greater social and economic benefits.

Thermohomeostasis: Changes in the temperature inside a cell change the way DNA works or how protein molecules work. To maintain the normal life activities of the human body and the necessary working efficiency, it is necessary to maintain a relative thermal balance. In our analysis of 26 natural medicines around the world, we found that the basic diagnostic items vary widely, but temperature testing is the only common thing. Therefore, body temperature is the foundation of the foundation and the common foundation of modern medicine and all natural medicine.

“Yin and Yang” is the general outline of TCM's eight cardinal principles of syndrome differentiation. It is a choice entry point for TCM internationalization to define Yin and Yang by quantifying cold and heat. It is often said that if you do not distinguish Yin and Yang, you will be wrong. In other words, to distinguish Yin and Yang is to enter the threshold of TRADITIONAL Chinese medicine. In the modern scientific platform to build the intelligent monitoring of traditional Chinese medicine, the guidance of different physique of rehabilitation therapy, personalized living habits and different physique of beauty guidance.

Detecting temperature information difference of human body symmetry is an early diagnosis of functional asymmetry in human body symmetry. Symmetry and dissymmetry are the contradictory unity of physiology and pathology, and the root cause of constant changes in health and disease. Develop the intelligence of accurate measurement and analysis to form a unified standard, adding an effective monitoring method for the treatment of diseases. Moreover, temperature monitoring is non-invasive, with high accuracy and resolution, and easy to integrate with IT.

The theory of symmetrical balance can be used to evaluate the efficacy of various treatments or drugs. By monitoring the treatment about or using drugs before and after the change of temperature difference judgment or the pros and cons of drug treatment. If the temperature difference between the left and right becomes smaller after the implementation of the treatment plan or drug, it indicates that the plan is effective. After the implementation, the temperature difference between the left and right increased, indicating that the scheme was unfavorable to the patients.

The theory of symmetrical balance can be used to guide rehabilitation. The positive and negative temperature difference can be used to judge whether the body is hot and cold or cold and hot. The positive and negative temperature difference reflects the direction of cold and heat, while the absolute value reflects the degree of deviation. According to the principle of giving heat to the cold, giving cold to the hot, taking the corresponding physical therapy and food traits, can effectively promote the rehabilitation of patients.

### DESCRIPTION OF DRAWINGS

FIG. 1A. Symmetrical wireless thermometer.

FIG. 1B. Functional Diagram of symmetrical wireless thermometer.

FIG. 1C. Schematic diagram of a 24 h symmetrical wireless thermometer probe

FIG. 1D. Schematic diagram of a symmetrical wireless thermometer.

FIG. 1E. Instructions for using the 24 h symmetric wireless clinical thermometer.

FIG. 1F. Schematic diagram of temperature difference for symmetrical balance quantization platform using smartphone APP for 24 h operation.

FIG. 1G. Clinical prototype of a 24-hour symmetrical wireless clinical thermometer.

Picture 1H. The backs of sensor and wearable device.

FIG. 2A. Symmetric Bluetooth thermometer for COVID-19 for both ears and right and left armpits.

FIG. 2B. Symmetrical Bluetooth thermometer ready to transmit information for COVID-19.

FIG. 3A. Power-on screen of wireless symmetric thermometer.

FIG. 3B. Body temperature monitoring.

FIG. 3C. Historical data.

FIG. 3D. Symmetrical multi-channel sensor with accuracy of ±0:02˜0:03° C.

FIG. 4A. Graph showing changes in temperature difference of a patient over time of using the claimed method.

FIG. 4B. Comparison chart of bilateral body temperature.

FIG. 5A. Comparison between stress echocardiogram and symmetric body temperature of a subject.

FIG. 5B. Comparison of stress echocardiogram and symmetric body temperature of subjects.

FIG. 5C. Part 1 of the electrocardiogram of a subject using the present invention.

FIG. 5D. Part 2 of the electrocardiogram of a subject using the present invention.

FIG. 5E. Part 3 of the electrocardiogram of a subject using the present invention.

FIG. 5F. Part 4 of the electrocardiogram of the subject using the present invention.

FIG. 5G. Part 5 of the electrocardiogram of a subject using the present invention.

FIG. 6. Non-contact infrared rapid temperature difference detector on both sides.

FIG. 7A. Double-sided multi-channel temperature recorder system.

FIG. 7B. Block diagram of a bilateral multi-channel temperature recorder.

FIG. 7C. The real-time monitoring interface.

FIG. 7D. Bilateral temperature recording.

FIG. 8. Health Cabin.

FIG. 8A. Health cabin for hand type.

FIG. 8B. Health cabin for foot type.

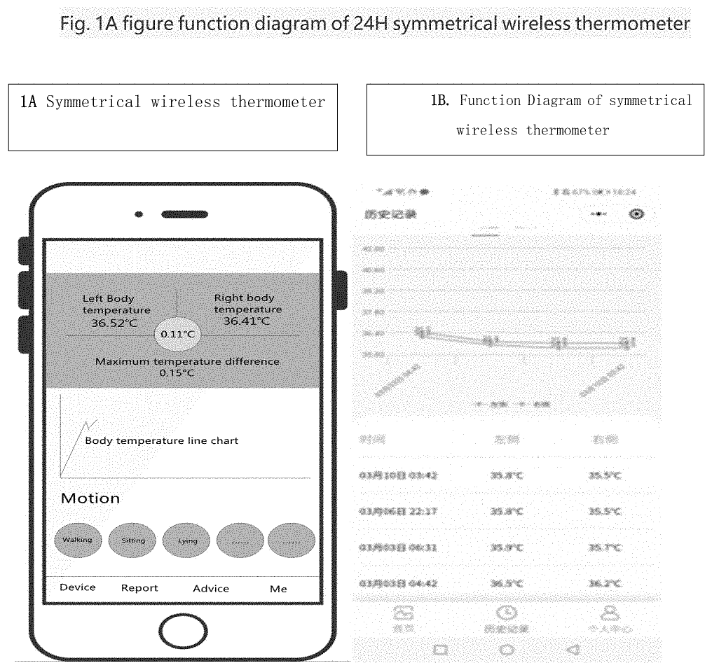

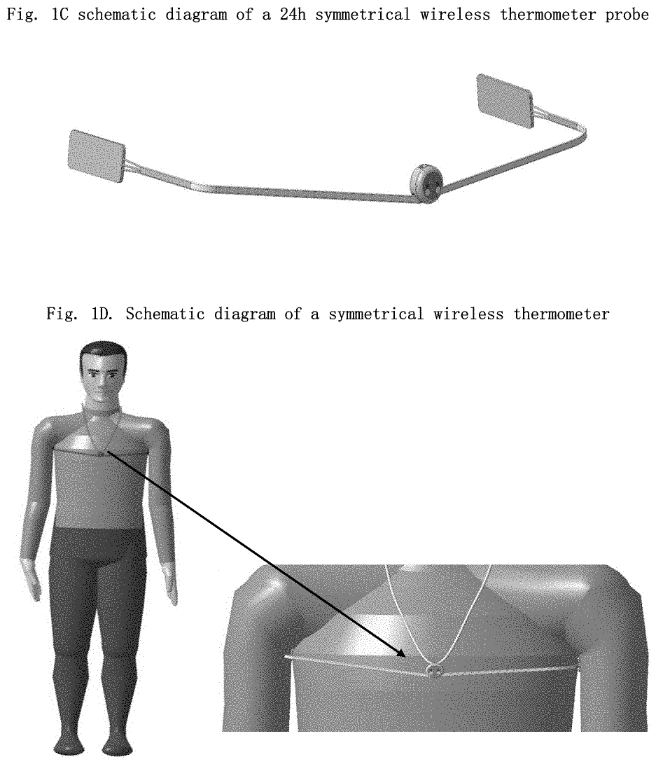

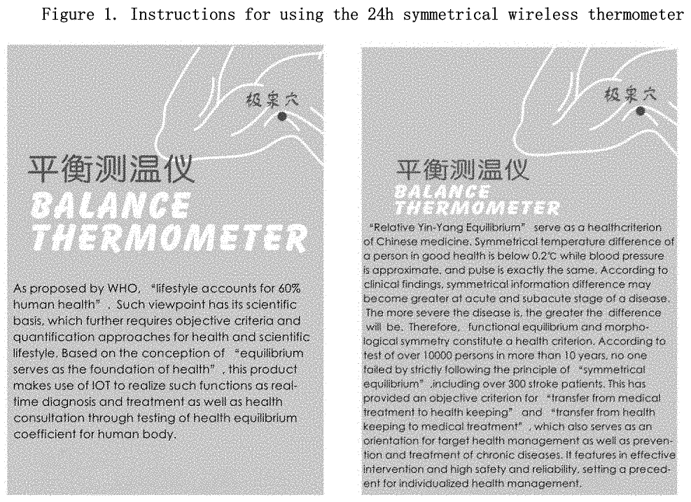

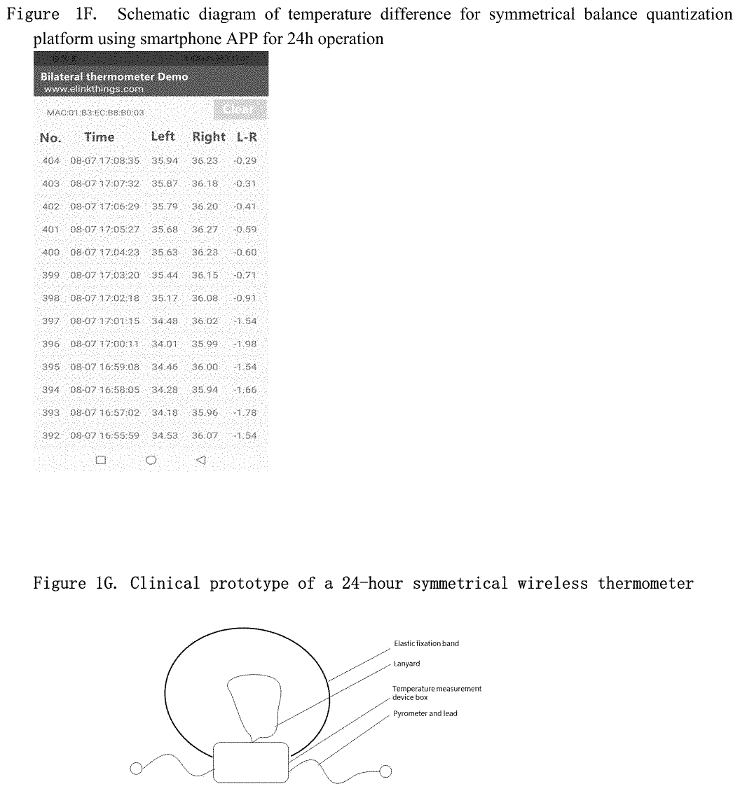

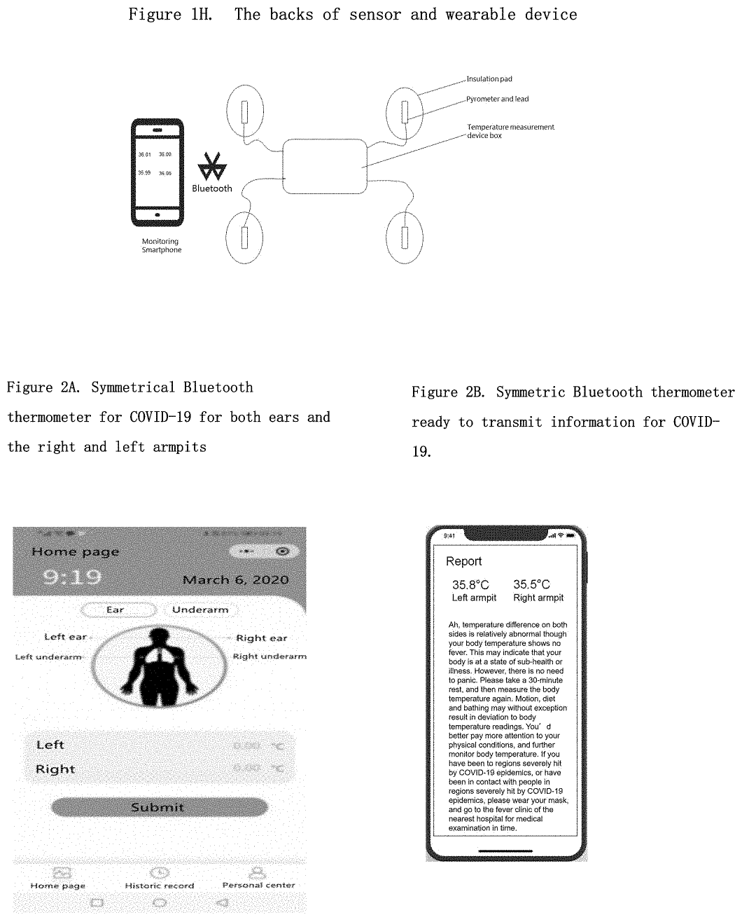

### DETAILED DESCRIPTION OF THE INVENTION

The present invention: Individualized remote health management system subjecting to dynamic observation, including data acquisition device for real-time measurement of such symmetrical physiological data as symmetrical body temperature, pulse, blood pressure, blood oxygen and blood glucose of users, and important physiological data on respiration and electrocardiograph as well as recording the environment, emotion, behavior and symptom information of users. The collected information is proceeded with real-time AI processing based on symmetrical balance principle to define physique of users, and stipulate individualized health intervention approaches. The symmetrical life information before and after health intervention is measured, the differences and physical coupling of which is used to seek the optimal individualized health intervention measures. The present invention is also expected to provide approaches for individualized health management and intervention using aforesaid systems.

Modern medicine has developed to the extent of uncanny workmanship. However, diseases and deaths incurred thereafter are increasing instead of decrease. Treatment of diseases by such natural medicines as traditional Chinese medicine is only an individual case. Such method may not be effective for treatment of the same patient, which is only provided with particularity other than universality. There are series of fine expressions in health field: Drink five cups of water per day; walk for 1000 steps; eat more vegetables and fruits. However, as indicated by practice, this may vary for different people and spatiotemporal conditions. It might be effective, ineffective, or even worse, which is unlikely to form criteria. Most of health care products take concept penetration or individual efficiency as the basis, which are in lack of individualized evidence basis. As a result, curative effect is instable, and reputation is unlikely to be established. This is just the doubt brought forth by individualization to medical health.

There are no two leaves or two persons that are exactly alike in the world. Time is passing all the time, and space is ever changing. This serves as an objective theoretical basis for essential individualization of life style and health intervention. However, promotion is also to be individualized. The coupling between standardization and individualization serves as an improvement to individualization.

The present invention involves the following specialized vocabularies. For other specialized vocabularies without specific definitions, their implications shall be in reference to that as generally understood by relevant ordinary staffs in the industry. Where there are no special indications, temperature measurement unit used in this thesis shall be centigrade (° C.). Symmetrical life information is arranged in an order of left before right in a standard mode. Symmetrical balance medicine is a new medicine mode, which serves as an emerging method for improvement of health and treatment of diseases in consideration of symmetrical life information of population, environment, life mode and individual differences. Health intervention scheme is to be customized based on symmetrical life information or physiology of patients. In other words, it means proceed with careful and accurate classification of cold and heat physiques of patients based on study of symmetrical life information discrepancies. Meanwhile, individualized health intervention is to be provided as per biological mechanism for cold and heat physiques. Lay emphasis on provision of different pertinent life styles and treatment modes for different patients. At the same time, stipulate uniform specifications for health management and treatment mode for people of the same physique (homo-subtype) so as to ensure coupling of individualization and standardization, and facilitate mass promotion. Create individualized diagnosis and treatment mode according to difference to symmetrical balance among people as well as differences to people in different natural and social environments.

Proceed with accurate test of symmetrical life information, and take information divergence as a parameter for such diagnostic judgments as judgment of physique, health status, life style and curative effect of health intervention. In this way, it is provided with an objective criterion.

Take basic symmetrical life information as a standard parameter for judgment of physique based on 10-day accurate test.

Body temperature and body temperature difference constitute the foundation of symmetrical balance, which serves as a yardstick for quantification of a variety of data on life activities. Its individualized theoretical basis is stated as follows: As disclosed by 242^{nd} American Chemical Society (ACS) National Meeting, variation to cell inside temperature may result in change of DNA working mode or operation mechanism of protein molecules. 2) Basic law of physics: Temperature is the statistical manifestation of average kinetic energy of gas molecules. Symmetrical body temperature difference reflects the difference to average kinetic energy of physiological functions of organs, tissues and cells of symmetrical parts, serving as the standardized basis for average kinetic energy of human molecules. 3) Mankind belongs to thermostatic animal. Dynamics of bilateral body temperature reflects its essential attributes. 4) Life styles such as clothing, food, shelter and transportation are also a representation of cold-heat effect in essence, which are directly associated with temperature. 5) Major or qualitative changes to physical conditions or state of an illness reflect correlation or causality based on temperature difference, P and BP. 6) Temperature serves as the greatest common divisor for each medical diagnosis. 7) Symmetrical body temperature makes use of two parameters as represented by space and time to constitute stereo information. 8) Cold-heat effect serves as the most major factor for impact of environment on people. 9) There are four seasons in a year, and the climate may be cold, hot, warm and cool; temperature difference has its orientation, and physique can be divided into coldness and warmness; motion-based heat dissipation is a vector; food can be cold, hot, warm and cool ones; emotion may be passionate and indifferent; such aspects are unified by coldness and warmness to set off health directionality. 10) The relative stability of all things attributes to functional balance and morphological symmetry, and the temperature difference is an individualized object of reference.

Collect offset data on left, right, upper and lower dynamic balance systems of individuals; The part above the thoracic diaphragm is defined as the upper part, and that below is defined as the lower part. According to the theory of traditional Chinese medicine and our clinical practice, the left side of human body corresponds to the upper part, and the right side corresponds to the lower part. Based on measurement of temperature on the left and right sides, orientation and “quantity” of the upper and lower temperature difference can be inferred. As “warm on the left and cool on the right” to human body is equivalent to “warm in the upper and cool in the lower”

various life modes and treatment approaches are used to make the upper cool and the lower warm, and vice versa. Diagnosis and treatment approaches of Western medicine and other natural therapeutics are to be integrated. On the basis of coldness and warmness, “uniform expression of physique/life style/environmental information and identical intervention method” are to be formed to provide corresponding health intervention measures and schemes. Discovery of health imbalance to users at the proper time aims to ensure coupling of individualization and standardization, and solve the problem of “what's the purpose” of data acquisition, which is an optimal method for prevention of diseases. It is also expected to “drive the market by taking curative effect as the pivot point”.

Based on real-time monitoring, we can quantify advantages and disadvantages brought forth by difference to temperature on both sides of human body to such life habits as lying, sitting, standing, running, defecation, eating, drinking and sleeping orientation as well as degrees of such advantages and disadvantages. Measure symmetrical life information on T, P, BP, blood oxygen and blood glucose as well as important individual life indicators, such as breathing and electrocardiograph. Balance trend is a reflection of health; whereas intensified asymmetry is a factor that is adverse to health. Life indicators approaching the center of normal value is a manifestation of a healthy life style, and vice versa. Such reaction is so obvious. From the point of view of real-time monitoring, such reflection will appear in about 1 minute. However, long-term effect of every individual item is to be defined with single parameter separation method through long-term comprehensive survey. Nevertheless, instantaneous survey has defined orientation of such mode as listed in the following table.

The symmetry of skeleton, organs, facial features and other organs represents a habitual practice for people to judge if physique or profile is symmetrical and normal; Western medicine believes that sympathetic nerve, parasympathetic nerve balance, psychological balance, acid-base balance and endocrine balance constitute important signs of health judgment According to traditional Chinese medicine, Yin-Yang balance is a symbol health judgment. Balance is ubiquitous inside body all the time. Accompanied by physiological, pathologic and environmental changes, variation to corresponding symmetrical information is to take place inside and outside body—symmetry-breaking difference (asymmetry). Presently, medical system is on the basis of morphology, and any disease can be discovered only in case of morphological changes. At quantitative change stage of health imbalance, there is in lack of objective criteria for judgment and rules for intervention measures. Symmetrical life information in different periods is a representation of different life states and clinical significance; orientation and value of information increment or decrement serve as objective criteria for health intervention.

Human body symmetry is relative and dynamic. Asymmetry is also in existence to any healthy person. The only difference lies in the fact that the degree is relatively low, and orientation is correct. This data serves as a criterion for health judgment. The contents of the patents and publications cited herein and the contents of documents cited in these patents and publications are hereby incorporated herein by reference to the extent permitted.

Without intending to be bound by any particular theory of operation, it is believed that the dynamic observation is an individualized expression of will. Take basal body temperature as a criterion to observe impact of space-time and intervention on curative effect according to space-time variation trend at present as well as before and after intervention so as to seek the optimal interventions, approaches and time. Symmetrical body surface information difference and data on its coupling with physique constitute the individualized and dynamic core technology. As acquisition time of body temperature transducer is 1 s-1 min, which is the same as change time of every motion of a person, it is applicable to real-time monitoring, supervision and intervention of various life modes, such as dinning, sports and mental state as well as symptoms and curative effect. In this way, it can realize transition from static test to dynamic observation to bring forth curative effect available for real-time quantification, and select prescriptions for diet, motion and psychological balance. Make use of different life styles, origination of intervention approaches and variation dynamics of life information in process to judge individualized advantages and disadvantages brought to the health as well as degrees. Establish digital measurement of impact of individualized life style and standard health intervention on the organism.

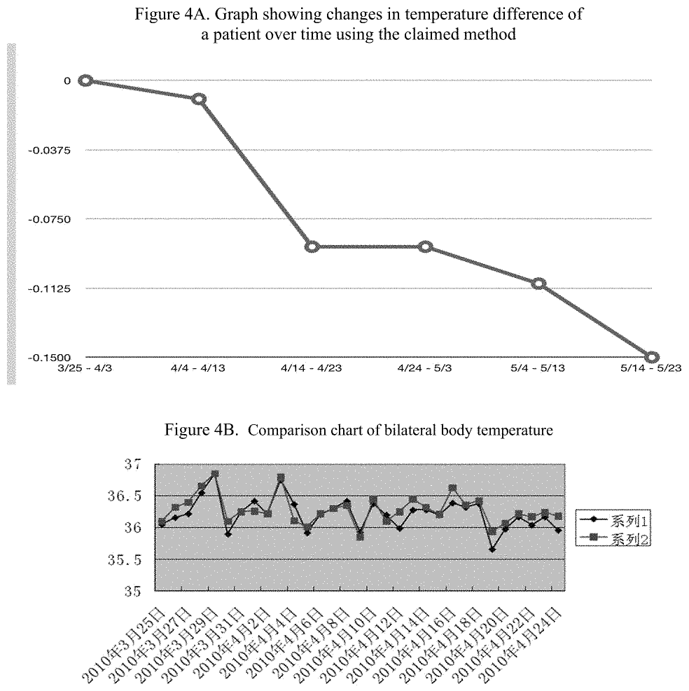

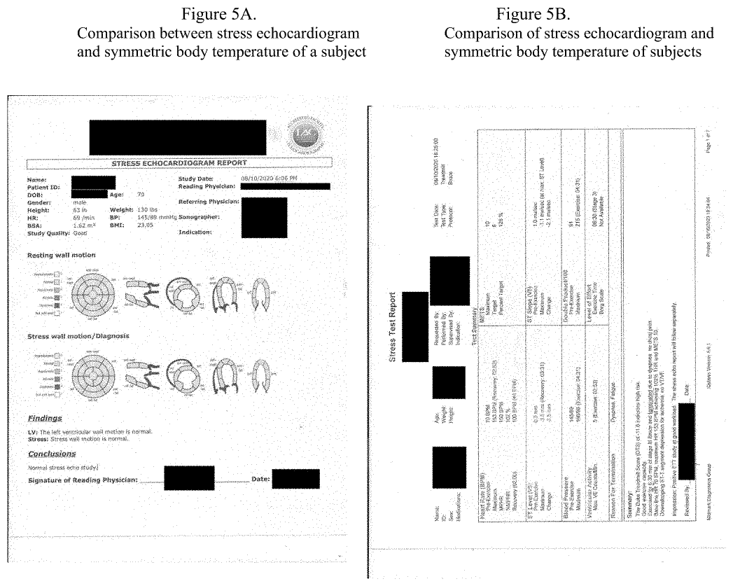

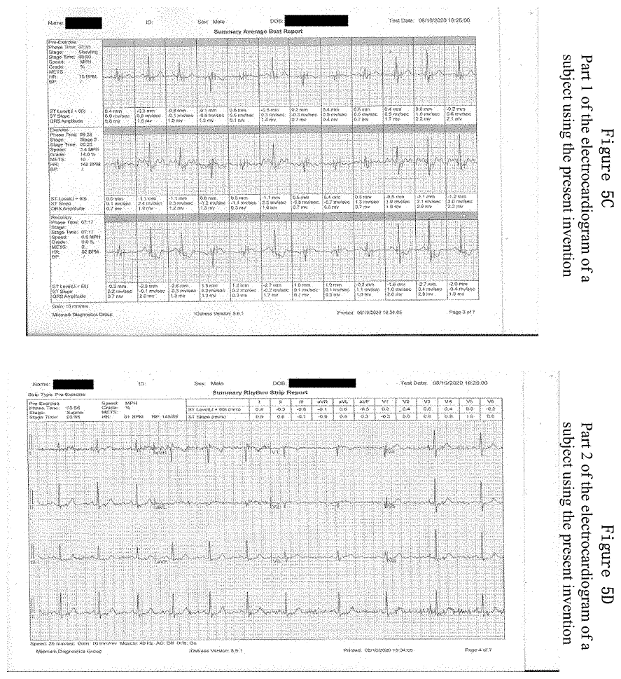

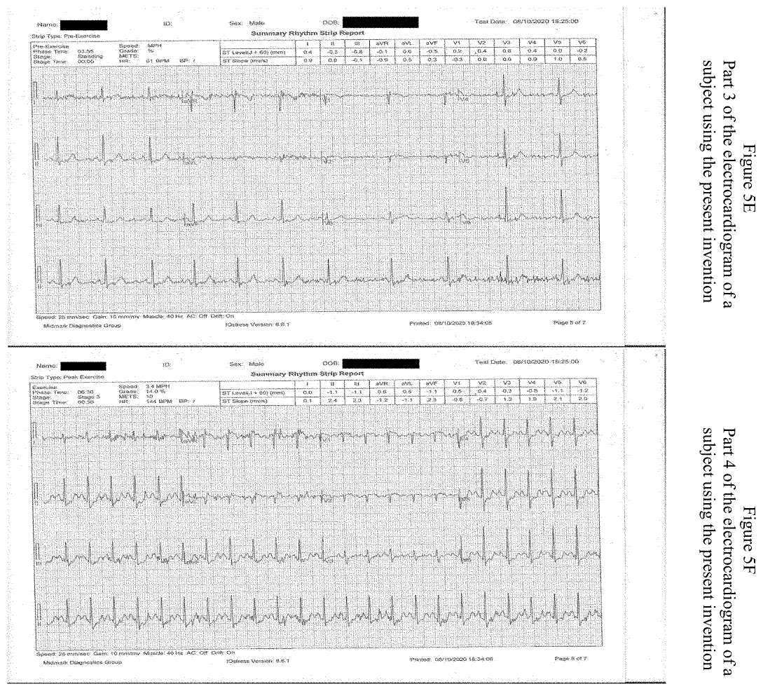

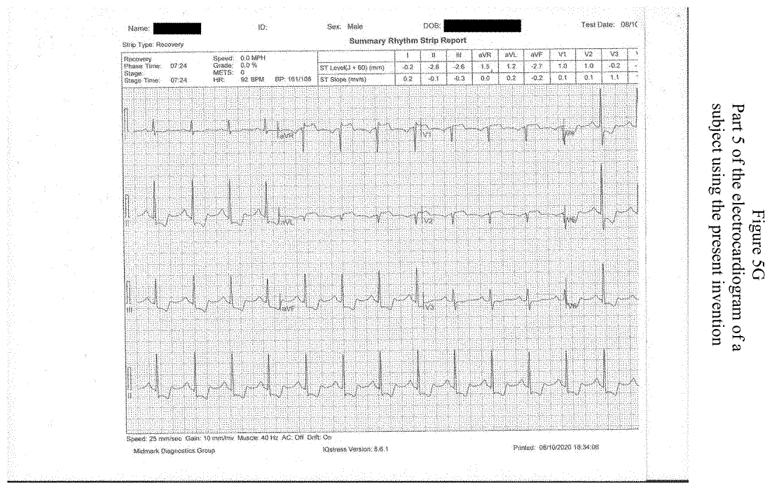

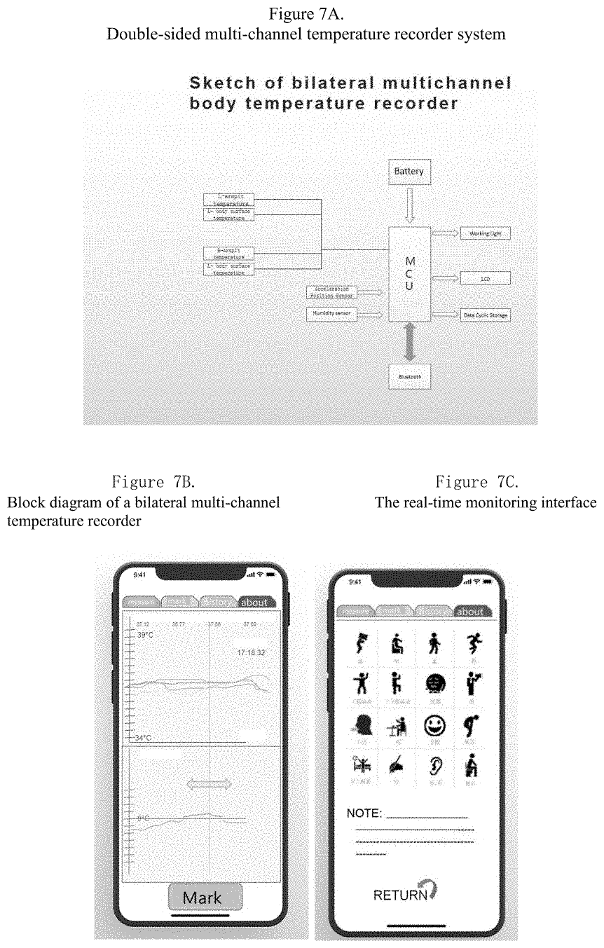

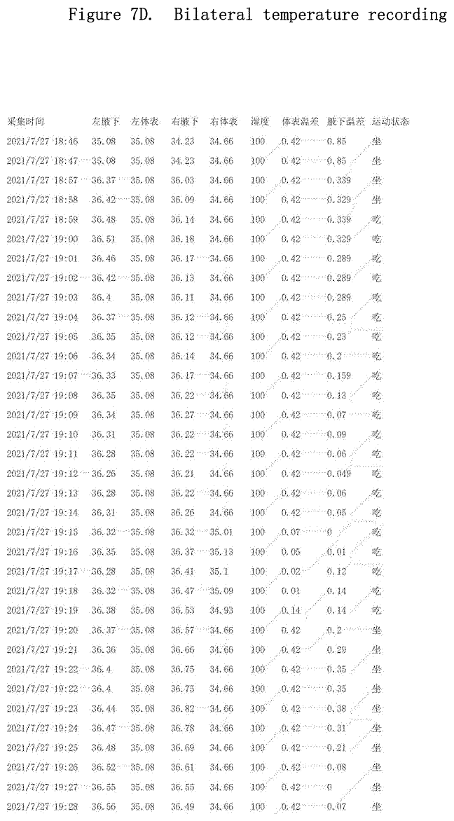

Set behavior/emotion input device FIG. 7D on the smartphone screen to input user's behavior and emotion information. Press “function box”/voice-operated switch. Observe dynamics to convert static test into big data on dynamic observation. Dynamic expression of symmetrical balance by IOT platform is stated as follows: 1) lying, 2) sitting, 3) walking, 4) running, 5) upper limb movement, 6) mixed movements, 7) listening and watching, 8) talking, 9) writing, 10) dinning, 11) fatigue, 12) joy and passion, 13) bitter suffered by us, 14) such modes as manual filling and voice input. Press function box when aforesaid behaviors/emotions start or occur, and press it again when finished. 1) press “lying” for sleeping, and press it again when you wake up; press “sitting” for sitting, and press it again for standing; 2) press “walking” for walking, and press it again for stop and rest; 3) press “running” for running, and press it again for stop (shift to walking or rest); 4) press it for upper limb movement, and press it again for stop; 5) function box for mixed movements of both upper and lower limbs, simultaneous movements of upper and lower limbs as well as dancing and so on; 6) before and after listening and watching, including listening to music, watching TV, entertainment programs and watching the scene of bustle; 7) before and after talking, including speaking, singing and speech; 8) writing: including that before and after writing articles and using computers; 9) before and after dinning; 10) fatigue, including uncomfortable feelings as occurred, such as dizziness, weakness and weary; 11) joy and passion occurred; 12) before and after defecation; 13) bitter occurred; 14) “function box” for input of items for current conditions in such modes as manual filling and voice input. (FIG. 4B)

In an embodiment, the data acquisition device in dynamic observation based individualized remote health management system according to the present invention further comprises a symptom input device. Acute and subacute attacks of chronic diseases may be accompanied by various symptoms. Such symptoms may result in dynamic variation to left and right body temperature, pulse, blood pressure, blood oxygen and blood glucose as well as differences among them. Set symptom input device on smartphone screen to facilitate input of user's symptoms and their intensity. Press “function box”/“voice operated” switch for dynamic observation of signs and dynamics of symptoms to acute and subacute attacks. Proceed with quantification of disease state and curative effect based on symptoms.

The user may press corresponding function box when aforesaid behaviors/emotions start or come to an end basically to acquire D-value to physiological data and environmental information at symmetrical part of body at the time of pressing. In this way, it will be easy to record interrelations among aforesaid movements, emotions, body temperature, temperature difference and variation to external environment as indicated to facilitate expression and computation in digital form. Thus, body temperature value and temperature difference are deemed to be their yardstick and object of reference. It is applicable to consider acquisition of continuous monitored body temperature value at start and completion, for example, every 20 s or three times per minute. By taking “walking” for 10 minutes for instance, it is applicable to monitor 30 data acquisition points in 10 minutes. This can further facilitate more detailed data analysis and monitoring of body dynamics. Software system comprises the following functions: A: receiving such life information as temperature via smartphone or computer, B: analysis of such life information as temperature, C: analysis of aforesaid ten movements as indicated, commencement of emotions, variation time as well as “basic” life information and information difference, intervention based life information at symmetrical part and information difference; this aims to establish a link between it and symmetrical life information and peripheral ambient temperature information so as to obtain analysis results; D: operating system software; time control system software. The following figure (see FIG. 4) is a diagram as plotted by me according to APP interface for simultaneous display of left and right temperature as well as real-time monitoring of temperature. APP may list out body temperature, ambient temperature and humidity of each period for background storage and design of AI algorithm for auto data analysis.

Establishment of numerous monitoring items does not mean inclusion of each person and item. IOT platform may request for use AI according to clinical demands when aforesaid behaviors/emotions start or come to an end basically. This aims to request for pressing of corresponding function box, and provide relevant intervention measures in time based on aforesaid designs, relevant countermeasures and life information data. Relevant information is to be fed back to customers via smartphone. For instance, when a customer with the symptom of “heat in the upper and cool in the lower” is running, the platform may request him to press “running” function box button to automatically record time, for example, 2019/8/16 15:22:47, L: 36.17° C., R: 36.05° C., temperature difference: +0.12 as well as ambient and adjacent temperature, humidity and meteorological conditions. 15 minutes after running, time recorded will be: 15:37:47, L: 36.26° C., R: 35.73° C., temperature difference: +0.53° C. The platform will send the following information to him according to AI computations: “Current exercise has an adverse impact on your health, you are recommended to stop it” and so on.

In an embodiment, equilibrium coefficient—orientation and value of dynamic symmetrical physiological data as well as orientation and value for increment and decrement of information difference as contained in dynamic observation-based health intervention approaches for physiological data serve as an objective criterion for health intervention. Use symmetrical balance AI for real-time processing, and compare equilibrium coefficient before and after specific behaviors to judge advantages and disadvantages brought by it to health individualization and corresponding degrees. Propose health intervention as per specific physique to realize individualized health management.

In an embodiment, the data acquisition device in dynamic observation based individualized remote health management system according to the present invention further comprises a “symptom” input device. Acute and subacute attacks of chronic diseases may be accompanied by various symptoms. Such symptoms may result in dynamic variation to left and right body temperature, pulse, blood pressure, blood oxygen and blood glucose as well as differences among them. Set symptom input device on smartphone screen to facilitate input of user's symptoms and their intensity. Press “function box” and “voice operated” switch for dynamic observation of signs and dynamics of symptoms to acute and subacute attacks. Proceed with quantification of disease state and curative effect based on symptoms.

In an embodiment, the data acquisition device in dynamic observation based individualized remote health management system according to present invention further comprises a synchronous ambient and adjacent environmental information acquisition device that is used to measure ambient and adjacent environmental information at the place where the user lives while using symmetrical balance acquisition device for measurement of physiological data. Such information is to be sent to IOT platform. It is applicable to quantify interrelation to ambient/adjacent environment at the said place, and express symmetrical balance dynamics to interrelation between dynamic and symmetrical physiological data difference affecting health and physiological state information corresponding to environmental information.

Take subjective symptoms of patients and measurement of symmetrical life information and its variation as individualized observation indicators. Take physiological and biochemical theory as a standard rule for orientation of movements and psychological equilibrium.

In an embodiment, equilibrium coefficient in health intervention method for dynamic observation of physiological data according to prevent invention further comprises semi-quantification of symptoms in case of acute and subacute attacks to chronic diseases. Taking the number from 1 to 10 as degree reference, it is 0, 10 and 5/10 respectively for insentience, unbearable feeling and moderate feeling. This can facilitate observation of quantitative relationship among physiological data, physiological data and symptom intensity to realize integration of quantification and semi-quantification.

Material-energy conversion has its orientation that serves as the direction of individualized quantification. Rules and direction of energy consumption and heat conversion are determined by body temperature difference, which serve as an individualized observation indicator. Intersection of symmetrical temperature curve and enlarged space represent significant or substantial changes to physical conditions and state of disease. Its evolution opposite to physical direction represents a health trend. Such test is more accurate in reflecting health as compared with mere measurement of body temperature and various life indicators in the hospital. Its correlation or causality serves as an effective approach for health monitoring. Trend and direction of dynamic symmetry breaking reflect the dynamics of relevant health changes; different physical types manifest the same direction for failed quantification of material-energy conversion, which can be used to determine advantages and disadvantages brought by relevant dynamics to health. Establish individualized digital measurement of health state: Acquire relevant data on symmetrical parts of human body at the same time, and extract life information contained under basically similar conditions. For instance, use both wrists to measure symmetrical pulse and blood pressure difference, measure left and right body temperature under both armpits and so on. Use transducer to acquire health state and ambient environmental conditions (temperature, humidity and so on) for comprehensive dynamic digitalization of data on the said person in this case: (1) Time, (2) climate, (3) region, (4) condition before and after exercise, (5) dinning habits, (6) condition before and after mood change, (7) condition before and after test, imaging and treatment, (8) condition before and after water drinking, defecation, smoking and wine drinking. Data acquisition is divided into that before and after basic symmetrical life information, movements, recovery and psychological activities in terms of time. Observe changes to symmetrical information difference before and after aforesaid contents to find corresponding correlation. Symmetrical life information in different periods represent different life conditions and clinical significance. Orientation and value for increment and decrement of information difference serve as objective criteria for health intervention. Constantly integrate information of emerging diseases to discover corresponding correlation. Changes to symmetrical life information difference of different time, space and physical conditions serve as an objective indicator for changes to time, space and physical conditions. Where significant or substantial changes take place to physical conditions or state of disease, information difference may reflect corresponding correlation and causality. Based on orientation and value of differences among them, it is applicable to make a precise judgment as to whether such life style, environmental factor, psychological activities and intervention approaches are favorable or harmful to you so as to identify potential hazards to health and unhealthy living habits. Orientation and value for increment and decrement to information difference serve as an objective criterion for health intervention.

To ensure better understanding of the prevent invention, it is applicable to refer to details of the following embodiments. Experienced professionals in relevant fields are requested to note that such preferred embodiments are only for explanation other than limitation on the prevent invention. For definitions in the prevent invention, please refer to “Claims” hereinafter.

Literatures or published articles are referred for the patent application. Literatures disclosed and articles published shall jointly constitute references for the application to facilitate more detailed description of the latest level of fields related to the prevent invention. Meanwhile, it is to be noted that the conjunctive word “comprise” and its synonyms have the implication of “all inclusive”. Therefore, other parts, methods or steps no mentioned are not to be excluded.

Without intending to be bound by any particular theory of operation, it is believed that rehabilitation orientation can be determined based on symmetrical information difference before and after rehabilitation. Physiotherapy and recovery are expected to enhance and restore physical conditions or physically handicapped functional capacity and living quality, which attach higher importance to dysfunction resulted from diseases. Functional restoration has its direction. As to the physique represented by heat in the upper and cool in the lower as proved by our clinical practice, functional restoration will inevitably result in temperature rise to lower body and temperature reduction to upper body to realize restoration of balance and vice versa. Match physical conditions with data before and after rehabilitation. Use health biomarker for quantification of rehabilitation curve of parameter group. Thus, it is applicable to define long-term maintenance of rehabilitation orientation and curative effect.

In an embodiment, Health intervention approaches in health management and diagnosis system driven by information on individualized remote health management system according to the present invention comprise recommendations on chills-fever balance, dinning habit, exercise methods and such rehabilitation approaches as physiotherapy, acupuncture, massage and so on. Orientation and value for increment and decrement in physiological data before and after health intervention serve as an objective criterion for rehabilitation intervention. Where equilibrium coefficient witnesses an increase after intervention, it means that such behavior is not applicable to patients, and such intervention is to be stopped. On the contrary, if equilibrium coefficient witnesses a decrease, it means that such intervention is favorable for patients, and can be continued.

In an embodiment, the diagnosis system of health management and diagnosis system driven by information on individualized remote health management system according to the present invention aims to measure symmetrical life information before and after use of different health intervention approaches, which is expected to seek the optimal individualized health intervention measures as per degree of coupling between relevant differences and physique.

In an embodiment, the data acquisition device in the dynamic observation based individualized remote health management system according to the present invention aims to realize symmetrical balancing of physique, dynamic data, rehabilitation, physiotherapy, targeted therapy and monitoring. This aims to seek orientation of targeted rehabilitation and AI for determination of prescription for targeted intervention to form integration of information acquisition, test, diagnosis and intervention.

Asymmetrical state of disease to any person may gradually become symmetrical accompanied by rehabilitation. However, it may subject to asymmetry in case of rehabilitation to a certain degree. The only difference is that it is just opposite to the physique orientation. Healing will raise symmetry from one level to a higher one, and thereby create new symmetry. symmetry to life information is a manifestation of health, and asymmetry opposite to the physique direction is the reflection of state of disease. New symmetry to state of disease is the reflection of health. In a certain quantity, asymmetry to life information opposite to physique direction is the reflection of gradual rehabilitation of disease.

Creation of metrological rehabilitation: Each patient is to be subject to quantified test of such symmetrical information as subjective symptoms, physical symptoms, musculoskeletal function, laboratory and imaging indicators, T, P, BP and so on before and after such rehabilitation approaches as physiotherapy, various therapies and acupuncture at gymnasium as well as massage. After that, the patient shall resort to mediated individual movements for correlation contrast before and after that. Actually, this aims to integrate qualitative test, semi-quantification and quantification with symmetrical quantification. However, relevant results can be expressed numerically to define rehabilitation directionality for real-time monitoring. This aims to seek treatment approaches that are most appropriate for the physique, which can provide quantified data for evolution and statistical inference, and enable to curative effect to make a leap. Impact of life style on health is natural. Test of symmetrical information difference before and after selection of a certain life style serves as an objective basis for establishment of a scientific life style.

Life depends on movements. This is the theoretical basis for establishment of various gymnasiums and playgrounds. As life comprises molecules that make up cells, life motion is just molecular motion. Everlasting irregular motion is physically called thermal motion. Variation to body temperature actually aims to judge the state of thermal motion, which can be deemed as quantified verification of advantages and disadvantages brought by motion to health for application to various sports occasions and gymnasiums. This aims to create conditions for syntropic and targeted fitness activities. From the perspective of individualization and physique, different people are requested to take part in different sports and fitness activities. Presently, there are numerous available sports equipment and increasing sports approaches. Motion is a vector, which has its directionality. Based on symmetrical balance monitoring, we can find what motions are favorable or unfavorable for you, what motions have synergistic effect and what motions are suitable for you? To make a simplified judgment, any data on balance trend and motion data in reverse inversion to physique represents a favorable reflection of your health based on results of symmetrical balance measurement. On the contrary, it will be an unfavorable reflection to you. This is expected to provide a clear objective criterion for gymnasium to ensure the health of its customers.

In an embodiment, the data acquisition device in the dynamic observation based individualized remote health management system according to the present invention further comprises an objective basis for judgment as to if life style/habit is correct. The conception proposed by WHO as to 60% lifestyles determined by health has been deeply rooted in the hearts of people. However, there is only speculative conceptual definition of lifestyle at present other than quantification dimension. Various conflicted opinions make people at a loss. What lifestyle is really appropriate for you? Due to the lack of quantification approaches and objective criterion, doctors may talk ambiguously regardless of patients who are at a loss. This will result in “homogenization” and conceptual disorder. Symmetrical equilibrium has solved this problem. 1. Make use of different lifestyles, start and completion of intervention approaches and variation trend of “equilibrium coefficient” of life information in process to judge advantage and disadvantages to health individualization and their degrees. Where difference to “balance coefficient” increases in the same direction, it means that this lifestyle is unfavorable for health, and vice versa. At the time of implementation, it is necessary to provide statistics on double significance to clinical efficacy. 2. Establish the biomarker parameter group of lifestyles. Take dietary rules of four properties and five tastes as proposed by Shen Nong's Herbal Classic and nutrition fundamentals of Western medicine as the basis for standard definition. 4. Take variation to symmetrical information before and after implementation of such interventions as motion, emotion and cultural arts as well as physique as individualized observation indicators. 5. As to aforesaid lifestyles, the use of symmetrical balance to verify and identify physical effect of information is favorable for improvement of targeted lifestyle.

Life styles such as clothing, food, shelter and transportation are also a representation of cold-heat effect in essence. The coupling between the nature and human physique will realize integration of health assessment, intervention and rehabilitation. Therefore, directionality and long-term nature of coupling constitute the core technology for individualized health management. 1. Actually, it is long-term evolution of incorrect lifestyle from quantitative change to qualitative one that brings into being all kinds of chronic diseases with such a high incidence rate. The only difference is its impact on health of human body is a gradual process that is just like dropping water through stone. Presently, precision medicine, the frontier of biomedical development, is benefited from difference to human genome, accurate diagnosis and targeted therapy. We use “unbalanced physical markers” formed by life information difference on the symmetrical body surface to supplement DNA sequence difference, and promote individualized medicine. Adherence to information difference principle, avoidance of astronomical number of gene differences and complicated sequencing are expensive and unavailable for real-time monitoring. Provide individualized prescriptions according to specific conditions of different people based on classification principles on cold, hot, warm and cool food and drugs, five properties and five flavors and channel tropism as stated in Shen Nong's Herbal Classic as well as coupling to physiques of coldness and warmness. 4. Provide prescriptions on individualized motion and psychological balance based on principles for adjustment of coldness and warmness by sports physiology and biochemistry. This aims to create a lifestyle that is coupled with physique, corresponding to quantification results and synchronized with seasonal changes, and achieve a harmonious unification. Convert original isolated treatment of patients into collaborative medicine, obtain valuable data for analysis and decision-making, and finally provide individualized intervention propositions. 5. Temporal and spatial changes, composite parameters and differences to personality may usually bring into being different situations. Just like a straight line between two points, a straight line is also to be generated through quantification of life information at two different time points. The difference between the two ends of this straight line is just its variation zone. Therefore, we shall measure symmetrical life information before and after intervention to lifestyle, and seek the optimal intervention measures according to relevant differences and physique coupling degree. Meanwhile, it is essential to establish correlation with physical examination indicators of Western medicine. Make use of advanced clinical statistic approaches to infer qualitative causality and balance trend model, and realize modernized and quantified diagnosis and rehabilitation. 6. Human body is fully unified, and symmetrical balance aims to realize comprehensive adjustment and control of lifestyle. In addition to good turn of major symptoms, such complications as hyperlipidemia, hyperglycemia and renal failure may also witness a good turn.

In an embodiment, individualized health management as stated in health intervention approaches for dynamic observation of physiological data according to present invention comprises provision of compatible prescriptions on diet, health care products and equipment, exercise methods and residence based on dynamic evaluation of physical signs, physique, lifestyle and environmental data as well as integration of human nature and clinical practice. This aims to realize individualized precise health management.

In an embodiment, behaviors as stated by health intervention methods for dynamic observation of physiological data according to the present invention comprises lifestyle, environmental factors, psychological activities, intervention approaches and so on. Where equilibrium coefficient increases after any behavior, it means that such behavior is not applicable to the patient; on the contrary, where equilibrium coefficient decreases, it means that such behavior is favorable for the patient, which can be continued.

Any normal person possesses a physiological mechanism and a psychological mechanism for self adjustment. Psychology refers to inner experience and external statements and actions. Psychology represents a process, a state, a function of the brain and a reflection of objective reality. The biggest difference among people lies in psychology. Despite of the fact that many diseases are represented by organ dysfunction and pathological changes, the underlying cause still lies in psychology. It is essential for individualized health management to quantify psychological factors. Quantitative analysis of psychological behavior is of vital importance. However, psychology is unavailable for quantification at present. Symmetrical equilibrium is the sole method for quantification and improvement of emotions worldwide.

Emotions include “excited”, “indifference”, “joy, anger, worry, thinking, sadness, fear and shock”, which are in correspondence to “coldness and warmness” relationship for quantification and issuance of corresponding prescriptions. Taking music therapy for instance, it is essential to measure left and right body temperature, pulse and blood pressure of patients in advance, record measurement time and data, and calculate equilibrium coefficient. Following music therapy, it is essential to measure body temperature on both sides, pulse and blood pressure of patients, record measurement time and data, and calculate equilibrium coefficient once again. After that, compare equilibrium coefficients before and after therapy. Where equilibrium coefficient increases, it means that the music used is not applicable to the patient, and is to be abandoned. On the contrary, if equilibrium coefficient decreases, it means that such music is favorable for the patient, which can be used continuously for therapy. Proceed with measurement at different period of a day, and define the period in which curative effect is the most satisfactory through comparison.

Take ten days as therapy unit to define the music and time that are most appropriate for the patient. Five internal organs according traditional Chinese medicine are both tangible and functional. Correlation between mind—five emotions: “joy, anger, thought, sadness, fear” and five internal organs is stated as follows: The heart, liver, spleen, lung and kidney govern joy, anger, thinking, worry and fear respectively. Radical emotions are harmful to five internal organs, and lesions to five internal organs may also result in abnormal emotions. Thus, morphology, function and mind are integrated. It is an enlightenment realized by integration of quantitative psychology and qualitative study. However, as there is no quantitative concept, only semi-quantification is available at most. Therefore, its development is slow. Corresponding quantification of “five internal organs”, “five emotions” and “coldness and warmness” will enable empirical psychology to make a leap.

According to clinical discovery of symmetrical balance to over 10000 persons in the past decade, symmetrical body surface information difference is associated with physique, curative effect and health state, which is directly related to emotions. Therefore, it is expected to lay down an empirical foundation for modern psychology. Five emotions are corresponding to left and right body temperature difference: As preliminarily discovered, “joy” may result in temperature rise on the left, and “melancholy” may result in temperature rise on the right. Thus, it is possible to provide a platform for the quantification of depression and suicidal tendency. We have issued corresponding emotional prescriptions for such patients during clinical treatments, proposed improvement approaches, and achieved good results. Severe depression is normally accompanied by syndrome of deficiency of vital energy in addition to stagnation of liver. The qualitative characteristic is fatigue, and semi-quantification characteristics is represented by fat tongue with tooth mark. This is due to the fact that people suffering from severe diseases may subject to local or systemic dysfunction. According to quantified measurement of pulse, right pulse is normally faster than left one. “Depression from stagnation and deficiency of vital energy” might be the underlying cause of depression. Victims of depression may become depressed and pessimistic, and may even lead to thoughts of suicide in severe case. In such case, temperature difference between both sides of the patient may exceed 0.40.6° C. accompanied by significant rise of platelet count and level of proteins, such as SAT1. Low spirit is the basic emotional symptom of depression. The more severe the it is, the greater the temperature difference between two sides will be.

According to principle of music-medicine homology, it is applicable to quantify symmetrical body surface information difference as well as advantages and disadvantages brought forth by music therapy by taking the physique as a guide before and after music therapy for the patient. This aims to realize individualized targeted rehabilitation, and establish a correlation with various scales for depression.

Victims of depression may have obvious clinical symptoms and even suicidal and violent thoughts when temperature difference between both sides is up to 0.45° C. This will provide scientific data support for prevention and treatment of depression and suicidal tendency, and realize integration of diagnosis and treatment of depression and suicidal tendency in terms of theory on physique of coldness and warmness.

The device can find the objective reference value directly related to the physiological and pathological process and the basis of life information, as well as the normal value range by physical, chemical, electronic and other means from the perspective of statistics and artificial intelligence (AI) for the diagnosis, therapy and efficacy of medical devices. These values are directly related to the life information of human body, which can increase the efficacy, reflect a more life-vital process and has more intuitive curative effect.

The newly developed multi-channel wireless symmetric thermometer adopts multiple NTC thermistors for temperature acquisition, such as L1 for the left axillary's temperature, L2 for the left shell temperature, R1 for the right axillary's temperature and R2 for the right shell temperature . . . and X for the on-body temperature. The temperature resolution is 0.01° C., and the temperature data is transmitted to the mobile APP through Bluetooth to form the core body temperature, shell temperature, left-right temperature difference curve and on-body temperature curve. The device will transmit the data to the workstation, introduce AI for processing and judgment, study the curve characteristics and combine them with the body movement state to obtain the physical condition of the patient and give treatment suggestions. With constant and ubiquitous monitoring, it will remind the patient of the pros and cons of the ongoing exercise, job and health status through SMS. And remind users to correct the wrong movement and work and lifestyle at any time.
A. The axillary's temperature reflects the core body temperature, but it changes with movement and turning, which will affect the continuity and stability of the monitoring data. The shell temperature probe is placed in the acromioclavicular joint and other body surface parts that are less affected by movement and posture changes. These parts will be affected by the conduction and convection of the outside air, resulting in the temperature difference between the axillary's temperature and shell temperature, but the temperature difference is relatively constant. (Figure x) The axillary's temperature cannot reflect the real temperature due to turning and movement, but the shell temperature is relatively constant. Therefore, the axillary's temperature can be inferred through the accumulation of big data and AI, to carry out monitoring continuously and accurately and ensure the correctness of diagnosis and the effectiveness of monitoring.
B. Input the user's behavioral action and emotional information; press the “Function Box”/“Voice Control” switch; observe the dynamic changes and transform real-time static testing into dynamic observation big data. Through more than half a year of research, we found that the start/stop time of movement can be judged by gyroscopes and acceleration sensors. Instead of pressing a button, this can be done automatically using AI. This will allow you to find out how the temperature difference changes and determine the health effects of sleeping on the left and right side and turning over. The health effects of other habitual left/right movements can also be readily addressed.

The core of our technology is to measure the temperature difference, and the accuracy of the temperature difference is required to reach 0.05-0.01° C. Therefore, the temperature sensor is developed to directly measure the temperature difference electromotive force and then calculate the temperature difference. Based on the thermal noise error limit theory, the patent technology designs a set of measurement methods to realize the measurement of weak signal (temperature difference), proposes a method to achieve the conventional temperature difference measurement index, and even proposes a method to approach the theoretical temperature difference measurement limit. The whole set of technology includes temperature sensing material, connection mode, weak signal digital processing method and temperature difference calibration method. To make a high-resolution measurement, the essence is to carry out weak signal measurement of voltage, current and resistance. (As shown in the figure) We installed a normal temperature sensor on the left side of the sensor. This temperature plus the temperature difference is the body temperature on the right. Direct measurement of temperature difference can improve accuracy and speed up transmission. The costs can also be reduced. We have built a prototype, and further research is required.

In one embodiment, the data acquisition device in the personalized remote health management system for dynamic observation of the invention also combines symmetrical balanced physique, dynamic data, modern medical instrument monitoring, and monitoring movement to find the direction of targeted therapy and optimal lifestyle.

A kind of equipment for directional control of the in vitro environment of human body—“Health Balance Cabin”, through the artificial external environment, regulates the cold and heat, water, electrolyte, interstitial pressure, etc. in different parts of the in vivo environment of human body, to order a disordered in vivo environment and achieve the balance of thermodynamics and physiology. By changing the in vivo and in vitro environment of human body—change the microenvironment of cancer cells and other disease states, to create an in vivo and in vitro environment that is favorable to the body and unfavorable to the disease state. (Table 5. Cool on top and hot on bottom “Balance Cabin” for bowel cancer treatment)

The “Health Balance Cabin” is a large container that you can sit in, with a partition set around your chest diaphragm. According to the treatment needs, the device is divided into type A and type B for patients with different physiques. Type A and type B “Health Balance Cabin” are similar in structure, and the difference is that the high and low temperature zones are opposite. Type A is used for patients cool on top and hot on bottom, and type B is used for patients hot on top and cool on bottom. For the new generation of intelligent “Health Balance Cabin”, the upper and lower temperature zones can be independently adjusted according to the prescription.

The internal space allows the patient to sit inside, and there is a transverse thermal insulation device (3) around the thoracic diaphragm to divide the internal space into upper and lower parts. Manual directional control can be carried out, including: a. Temperature: the top, bottom, left, right, front, back six directions can be set between 0° C.-50° C., and patients cold on top and hot on bottom can use devices (see Figure x) hot on top (1. with spectrum and other heating equipment) and cold on bottom (2. with a variety of refrigeration equipment) for free adjustment. The opposite is true for patients hot on top and cold on bottom; b. temperature; c. wind power and wind direction; d. concentration and proportion of air components, such as oxygen; e. voice (including music, stories, talk shows, soap operas, etc. that make people happy, sad, angry, sad, worried, thinking, frightened and afraid); f. vision (including plays and movies, etc. that make people happy, sad, angry, sad, worried, thinking, frightened and afraid); g. the amount of trace elements, natural animal and plant products, and chemicals in contact with the human body; h. contact tightness, pressure and quantity of the above items.

The above items can be easily controlled by the customer or the operator according to our guidance and their own feelings with a button or remote control, to influence and regulate the body cold, heat, water electrolyte, interstitial pressure and other in vivo environment and the mood, change the surrounding environment, keep the patient out of contact with undesirable irritants and only come in contact with the in vitro environment that is beneficial to the body. For control of different items, the wearable 5-channel sensors. The user is measured to determine their physical fitness, can be divided into type A and type B for patients with different physical physiques according to the treatment needs.

### Example 1. Symmetrical Balance-Based Quantification of Curative Effect of Traditional Chinese Medicine

Treatment of a positively imbalanced patient with heat on the top and cold on the bottom, with persistent pain of 5-7 on a scale of 10. According to the symmetrical balance syndrome differentiation treatment, taking Chinese medicine for 20 days, the overall body temperature gradually to the health of the transformation, the overall body temperature to the negative transformation. In the first 7 days, 40 minutes after taking Chinese medicine each time, the negative value decreased on the other hand. After adjustment, 13 days after taking Chinese medicine, the negative value increased, the pain was significantly improved, and the negative value increased and weight loss, which double confirmed the accuracy of the drug.

TABLE 1
Data on Curative Effect of Symmetrical Balance Based Diagnosis and Treatment
Based on Overall Analysis of Illness and Patient's Condition

| | | Body temperature | | | | | |
|---|---|---|---|---|---|---|---|
| | | before oral | | | 40 min after oral | | |
| | Date | administration (° C.) | | | administration (° C.) | | |
| | 2010 | L | R | Difference | L | R | Difference |
| 10-kidney | 3/25 | 36.05 | 36.1 | −0.05 | 36.07 | 36.07 | 0 |
| 1-gallbladder | 3/26 | 36.16 | 36.32 | −0.16 | 36.37 | 36.45 | −0.08 |
| 2-liver | 3/27 | 36.22 | 36.4 | −0.18 | 36.3 | 36.38 | −0.08 |
| 3-small | 3/28 | 36.55 | 36.66 | −0.11 | 36.56 | 36.66 | −0.1 |
| intestine | | | | | | | |
| 4-heart | 3/29 | 36.85 | 36.85 | 0 | 36.8 | 36.98 | −0.18 |
| 5-stomach | 3/30 | 35.9 | 36.1 | −0.2 | 36.35 | 36.49 | −0.14 |
| 6-spleen | 3/31 | 36.25 | 36.25 | 0 | 36.97 | 36.81 | 0.16 |
| 7-large | 4/1 | 36.42 | 36.26 | 0.16 | 35.64 | 35.58 | 0.06 |
| intestine | | | | | | | |
| 8-lung | 4/2 | 36.22 | 36.22 | 0 | 36.3 | 36.45 | −0.15 |
| 9-bladder | 4/3 | 36.75 | 36.8 | −0.05 | 36.86 | 36.89 | −0.03 |
| | | 36.337 | 36.396 | −0.06 | 36.422 | 36.476 | −0.05 |
| 10-kidney | 4/4 | 36.37 | 36.11 | 0.26 | 36.15 | 36.25 | −0.1 |
| 1-gallbladder | 4/5 | 35.92 | 36.01 | −0.09 | 36.26 | 36.33 | −0.07 |
| 2-liver | 4/6 | 36.21 | 36.22 | −0.01 | 36.36 | 36.64 | −0.28 |
| 3-small | 4/7 | 36.3 | 36.3 | 0 | 36.08 | 36.15 | −0.07 |
| intestine | | | | | | | |
| 4-heart | 4/8 | 36.42 | 36.35 | 0.07 | 36.76 | 36.75 | 0.01 |
| 5-stomach | 4/9 | 35.93 | 35.85 | 0.08 | 36.51 | 36.65 | −0.14 |
| 6-spleen | 4/10 | 36.38 | 36.45 | −0.07 | 36.35 | 36.48 | −0.13 |
| 7-large | 4/11 | 36.2 | 36.1 | 0.1 | 36.31 | 36.28 | 0.03 |
| intestine | | | | | | | |
| 8-lung | 4/12 | 35.99 | 36.25 | −0.26 | 36.3 | 36.3 | 0 |
| 9-bladder | 4/13 | 36.28 | 36.45 | −0.17 | 36.6 | 36.82 | −0.22 |
| | | 36.2 | 36.209 | −0.01 | 36.368 | 36.465 | −0.1 |
| 1-gallbladder | 4/14 | 36.28 | 36.32 | −0.04 | 36.71 | 36.6 | 0.11 |
| 2-liver | 4/15 | 36.2 | 36.21 | −0.01 | 36.49 | 36.56 | −0.07 |
| 3-small | 4/16 | 36.39 | 36.63 | −0.24 | 35.97 | 36.1 | −0.13 |
| intestine | | | | | | | |
| 4-heart | 4/17 | 36.32 | 36.36 | −0.04 | 36.22 | 36.37 | −0.15 |
| 5-stomach | 4/18 | 36.38 | 36.43 | −0.05 | 36.52 | 36.57 | −0.05 |
| 6-spleen | 4/19 | 35.66 | 35.94 | −0.28 | 36.22 | 36.42 | −0.2 |
| 7-large | 4/20 | 35.98 | 36.07 | −0.09 | 36.23 | 36.37 | −0.14 |
| intestine | | | | | | | |
| 8-lung | 4/21 | 36.17 | 36.22 | −0.05 | 36.36 | 36.51 | −0.15 |
| 9-bladder | 4/22 | 36.04 | 36.17 | −0.13 | 36.11 | 36.06 | 0.05 |
| | | 36.1578 | 36.2611 | −0.1 | 36.3144 | 36.3956 | −0.08 |
| 10-caprine | 4/23 | 36.17 | 36.24 | −0.07 | 36.54 | 36.67 | −0.13 |
| kidney | | | | | | | |
| 1-gallbladder | 4/24 | 35.96 | 36.18 | −0.22 | 36.26 | 36.44 | −0.18 |
| 2-liver | 4/25 | 36.05 | 36.17 | −0.12 | 36.45 | 36.2 | 0.25 |
| 3-small | 4/26 | 36.3 | 36.3 | 0 | 36.3 | 36.3 | 0 |
| intestine | | | | | | | |
| 4-heart | 4/27 | 35.8 | 35.92 | −0.12 | 36.46 | 36.26 | 0.2 |
| 5-stomach | 4/28 | 36.7 | 36.65 | 0.05 | 36.22 | 36.36 | −0.14 |
| 6-spleen | 4/29 | 36.6 | 36.5 | 0.1 | None | None | N/A |
| 7-large | 4/30 | 36.54 | 36.73 | −0.19 | None | None | N/A |
| intestine | | | | | | | |
| 8-lung | 5/1 | 36.11 | 36.16 | −0.05 | 36.23 | 35.76 | 0.47 |
| 9-bladder | 5/2 | 35.1 | 35.3 | −0.2 | 35.7 | 35.9 | −0.2 |
| | | 36.13 | 36.21 | −0.08 | 35.97 | 35.83 | 0.14 |
| 10-caprine | 5/3 | 36.29 | 36.33 | −0.04 | 35.8 | 36 | −0.2 |
| kidney | | | | | | | |
| 1-gallbladder | 5/4 | 36.1 | 36.3 | −0.2 | 35.4 | 35.6 | −0.2 |
| 2-liver | 5/5 | 36.36 | 36.5 | −0.14 | 36.55 | 36.55 | 0 |
| 3-small | 5/6 | 36.9 | 37 | −0.1 | 36.74 | 36.9 | −0.16 |
| intestine | | | | | | | |
| 4-heart | 5/7 | 36.12 | 36.23 | −0.11 | 36.79 | 36.82 | −0.03 |
| 5-stomach | 5/8 | 35.98 | 36.09 | −0.11 | 36.77 | 36.98 | −0.21 |
| 6-spleen | 5/9 | 36.2 | 36.28 | −0.08 | 37.05 | 37.1 | −0.05 |
| 7-large | 5/10 | 36.27 | 36.32 | −0.05 | 36.52 | 36.68 | −0.16 |
| intestine | | | | | | | |
| 8-lung | 5/11 | 36.27 | 36.27 | 0 | 36.52 | 36.61 | −0.09 |
| 9-bladder | 5/12 | 36.1 | 36.17 | −0.07 | 36.7 | 36.77 | −0.07 |
| | | 36.259 | 36.349 | 0.09 | 36.484 | 36.601 | −0.12 |
| 10-caprine | 5/13 | 35.97 | 36.12 | −0.15 | 36.23 | 36.34 | −0.11 |
| kidney | | | | | | | |
| 1-gallbladder | 5/14 | 36.83 | 36.96 | −0.13 | 36.95 | 37.05 | −0.1 |
| 2-liver | 5/15 | 36.46 | 36.59 | −0.13 | 36.85 | 37.1 | −0.25 |
| 3-small | 5/16 | 36.21 | 36.48 | −0.27 | 36.5 | 36.74 | −0.24 |
| intestine | | | | | | | |
| 4-heart | 5/17 | 36.33 | 36.44 | −0.11 | 36.36 | 36.53 | −0.17 |
| 5-stomach | 5/18 | 36.04 | 36.35 | −0.31 | 36.74 | 37 | −0.26 |
| 6-spleen | 5/19 | 36.48 | 36.6 | −0.12 | 36.5 | 37 | −0.5 |
| 7-large | 5/20 | 36.68 | 36.88 | −0.2 | 36.35 | 36.62 | −0.27 |
| intestine | | | | | | | |
| 8-lung | 5/21 | 36.36 | 36.4 | −0.04 | 37.12 | 37.27 | −0.15 |
| 9-bladder | 5/22 | 37.12 | 37.27 | −0.15 | 36.47 | 36.57 | −0.1 |
| | | 36.448 | 36.609 | −0.16 | 36.607 | 36.822 | −0.22 |
| 10-caprine | 5/23 | 36.69 | 36.69 | 0 | 36.89 | 37.17 | −0.28 |
| kidney | | | | | | | |
| 1-gallbladder | 5/24 | 36.52 | 36.84 | −0.32 | Withdrawal | Take | N/A |
| | | | | | | Common | |
| | | | | | | Goldenrop | |
| 2-liver | 5/25 | 36.55 | 36.62 | −0.07 | Withdrawal | Take | N/A |
| | | | | | | Common | |
| | | | | | | Goldenrop | |

### Example 2. A Symmetrical Balance can be Used to Identify the Effectiveness of Chemical Drugs

Chemo chemotherapy can turn physique of a colorectal cancer patient from the positive to the negative despite of extremely short maintenance period. The following table records the measurement results of aforesaid American colorectal cancer patient of white race during chemotherapy at New Jersey Cancer Hospital on 2009, Sep. 2-4. Positive difference reduced by 0.15° C., 0.6° C. and 0.3° C. respectively following the 1st, 2nd and 3rd chemotherapy. As proved by symmetrical balance theory, chemotherapy is effective despite of extremely short maintenance period—1 day.

TABLE 2
Measurement Results during Chemotherapy

| | | | L- | R- | Temperature | |
|---|---|---|---|---|---|---|
| Date | Time | | temperature | temperature | difference | Remark |
| 2-Sep | 6:16 | am | 36.6° C. | 36.45° C. | 0.15° C. | chemo today |
| 2-Sep | 11 | pm | 36.65 | 36.65 | 0 | Positive difference |
| | | | | | | reduced by 0.15° C. |
| | | | | | | following chemotherapy. |
| 3-Sep | 6 | am | 36.15 | 36.1 | 0.05 | nausea, tired, 2 nd |
| | | | | | | chemotherapy |
| 3-Sep | 10 | pm | 36.15 | 36.7 | −0.55 | out of control emotions, |
| | | | | | | conversion to the |
| | | | | | | negative by |
| | | | | | | 0.6° C. |
| 4-Sep | 6 | am | 36.65 | 36.4 | 0.25 | chemo affecting me 3^{rd} |
| | | | | | | chemotherapy |
| 4-Sep | 10 | pm | 36.75 | 36.8 | −0.05 | chemo affecting me, |
| | | | | | | conversion to the |
| | | | | | | negative by 0.3° C. |
| 5-Sep | 7 | am | 35.9 | 36.15 | −0.25 | sweated all night |
| 5-Sep | 10:30 | pm | 36.9 | 36.4 | 0.5 | calmer, more energy |
| 6-Sep | 6:44 | am | 36.9 | 36.4 | 0.5 | |
| 6-Sep | 9:40 | pm | 37 | 36.95 | 0.05 | |
| 7-Sep | 7:10 | am | 36.4 | 36.45 | −0.05 | |
| 7-Apr | 9:30 | pm | 36.85 | 36.9 | −0.05 | |
| 8-Sep | 6:21 | am | 36.45 | 36.55 | −0.1 | |
| 9-Sep | 10:30 | pm | 37 | 36.95 | 0.05 | sweated heavily |
| | | | | | | throughout night |
| 9-Sep | 6:41 | am | 36.25 | 36.15 | 0.1 | |

Combination of symmetrical body temperature with modern medical instruments may establish a data correlation for precise definition of intervention orientation and quantity, which can improve curative effect without incurring damages, pain and side effect.

### Example 3. Symmetrical Balance-Based Quantification of Curative Effect of Acupuncture Therapy

Function restoration is directional. As proved by us during clinical practice, for physique that is heat in the upper and cool in the lower, functional restoration may inevitably temperature rise to upper body and temperature decrease to lower body. It is to be balanced before restoration and vice versa. Matching between physique and data difference before and after acupuncture is available for definition of therapy orientation, which is expected to find a method for improvement of curative effect.

TABLE 3
Data on Symmetrical Balance Based Quantitative Acupuncture Therapy

| | | | | | | Blood | | |
|---|---|---|---|---|---|---|---|---|
| | | | | | | Pressure | | |
| Date | Temperature, ° C. | | | Pulse/min | | (mmHg) | | Sleep |
| 2021 | L | R | Diff. | L | R | L | R | quality |
| 15-Jan | 36.16 | 36.15 | 0.01 | 52 | 54 | | | 2 |
| Data | | | 0 | | | | | 10 |
| sheet 1 | | | | | | | | |
| 16-Jan | 36.1 | 36.3 | −0.2 | 56 | 55 | | | 5 |
| 17-Jan | 35.72 | 36.18 | 0.46 | 53 | 55 | | | 5 |
| 18-Jan | 36.2 | 36.25 | 0.05 | | | | | 4 |
| 19-Jan | 35.95 | 35.8 | 0.15 | | | | | 3 |
| 20-Jan | 35.9 | 36.12 | 0.22 | | | | | 5 |
| 21-Jan | 36.38 | 36.27 | 0.11 | | | | | 5 |
| 22-Jan | 35.96 | 36.03 | 0.07 | | | | | 4 |
| 23-Jan | 36.1 | 36.39 | 0.29 | | | | | 5 |
| 24-Jan | 35.95 | 35.8 | 0.15 | | | | | 5 |
| 25-Jan | 36.2 | 36.3 | -0.1 | | | | | 6 |
| 26-Jan | 36.15 | 36.2 | 0.05 | | | | | 6 |
| 27-Jan | 36.23 | 36.33 | -0.1 | | | | | 2 |
| 28-Jan | 35.59 | 35.75 | 0.16 | | | | | 2 |
| 29-Jan | 36.25 | 36.26 | 0.01 | | | | | 5 |
| 30-Jan | 36.04 | 35.9 | 0.14 | | | | | 5 |
| 31-Jan | 35.92 | 36 | 0.08 | | | | | 5 |
| 1-Feb | 36.1 | 36.33 | 0.23 | | | | | 4 |
| | 36.04 | 36.13 | 0.09 | | | | | 40 |
| 2-Feb | 36.07 | 36.17 | −0.1 | | | | | |
| 3-Feb | 36.05 | 36.29 | 0.24 | | | | | 4 |
| 4-Feb | 36.05 | 36.07 | 0.02 | | | | | 2 |
| 5-Feb | 36.55 | 36.6 | 0.05 | | | | | 0 |
| 6-Feb | 36.22 | 36.27 | 0.05 | | | | | 2 |
| 9-Feb | 36.17 | 36.3 | 0.13 | 72 | 70 | 100/ | 109/ | 6 |
| | | | | | | 81 | 78 | |
| 10-Feb | 36.2 | 36.25 | 0.05 | 64 | 64 | 97/ | 99/ | 8 |
| | | | | | | 69 | 73 | |
| 11-Feb | 36.01 | 36.04 | 0.03 | 60 | 65 | 98/ | 99/ | 1 |
| | | | | | | 71 | 70 | |
| 12-Feb | 36.19 | 36.1 | 0.09 | 62 | 65 | 94/ | 92/ | 5 |
| | | | | | | 73 | 58 | |
| | 36.09 | 36.17 | 0.06 | | | | | |
| 13-Feb | 36.4 | 36.17 | 0.23 | 63 | 60 | 98/ | 95/ | 7 |
| | | | | | | 62 | 65 | |

### Example 4. Comparison Between Symmetrical Balance Based Quantified Therapy and Curative Effect of Renowned Acupuncture Doctor

Treating rectal cancer patients with little signs of life. Body temperature in 10 days has decreased. Temperature difference has increased by −0.01° C., and temperature has turned to positive by 0.01° C. (See Table 4).

TABLE 4
Acupuncture Free Life Information on Rectal Cancer Cases

| Date | Temperature of Armpit, ° C. | | | Pulse, #/min | | Blood Pressure | | |
|---|---|---|---|---|---|---|---|---|
| 2021 | L | R | Dif. | L | R | L | R | Dif. |
| 15-Feb | 36.14 | 36.22 | 0.08 | 60 | 60 | 103/65 | 107/65 | 2 |
| 16-Feb | 36.36 | 36.3 | 0.06 | 57 | 60 | 98/61 | 98/64 | 7 |
| 17-Feb | 36.41 | 36.45 | 0.04 | 60 | 65 | 103/69 | 98/65 | 7 |
| 18-Feb | 36.3 | 36.3 | 0 | 60 | 65 | 103/71 | 100/73 | 0 |
| 19-Feb | 36.37 | 36.38 | −0.01 | 80 | 83 | 119/84 | 120/83 | 5 |
| 20-Feb | 36.22 | 36.42 | −0.2 | 60 | 60 | 106/73 | 105/69 | 7 |
| 21-Feb | 36.17 | 36.25 | −0.08 | 66 | 72 | 106/70 | 100/73 | 7 |
| 22-Feb | 36.51 | 36.41 | 0.1 | 65 | 65 | 98/65 | 99/66 | 0 |
| 23-Feb | 36.37 | 36.17 | 0.2 | 66 | 65 | 103/63 | 99/57 | 6 |
| 24-Feb | 36.36 | 36.55 | −0.19 | 78 | 65 | 105/65 | 102/65 | 6 |
| 25-Feb | 36.25 | 36.15 | 0.1 | 63 | 68 | 102/65 | 100/68 | 6 |
| 2,15-25 | 36.315 | 36.327 | −0.01 | 65 | 66.2 | | | |
| 26-Feb | 36.46 | 36.3 | 0.16 | 61 | 63 | 100/72 | 106/67 | 7 |
| 27-Feb | 36.23 | 36.15 | 0.08 | 60 | 62 | 98/65 | 98/68 | 5 |
| 28-Feb | 36.1 | 36 | 0.1 | 61 | 58 | 92/65 | 94/64 | 3 |
| 1-Mar | 36.4 | 36.54 | −0.14 | 72 | 70 | 99/67 | 99/66 | 2 |
| 2-Mar | 36.2 | 36.22 | −0.02 | 61 | 66 | 102/71 | 100/70 | 7 |
| 3-Mar | 36.37 | 36.4 | −0.03 | 62 | 60 | 102/76 | 107/76 | 7 |
| 4-Mar | 36.3 | 36.4 | −0.1 | 60 | 64 | 97/73 | 99/71 | 2 |
| 6-Mar | 36.08 | 36.15 | −0.07 | 61 | 65 | 99/70 | 97/70 | 2 |
| 7-Mar | 36.51 | 36.45 | 0.06 | 66 | 64 | 108/78 | 105/74 | 2 |
| 8-Mar | 36.28 | 36.32 | −0.04 | 62 | 60 | 107/75 | 104/75 | 8 |
| 9-Mar | 36.31 | 36.21 | 0.1 | 63 | 65 | 104/78 | 102/70 | 2 |
| 2/26- | 36.29 | 36.29 | 0.01 | 63 | 63.5 | | | |
| 3/9 | | | | | | | | |
| 10-Mar | 36.17 | 36.19 | −0.02 | 67 | 71 | 112/77 | 111/76 | 6 |
| 11-Mar | 36.41 | 36.25 | 0.16 | 67 | 71 | 112/77 | 111/76 | 6 |
| 12-Mar | 36.42 | 36.29 | 0.13 | 73 | 75 | 114/73 | 107/75 | 6 |

### Example 5. Acupuncture is Effective for Treatment of Tumors as Proved by Symmetrical Data

Following the commencement of test for symmetrical life information, the patient suffering from chronic disease took medicine and received injection every day. Furthermore, her spouse contacted me for treatment, and the patient was in a high spirit. Therefore, equilibrium coefficient of body temperature difference decreased. Temperature difference was so limited, and was maintained at a positive value. Acupuncture was carried out by a renowned acupuncture doctor in Shenzhen. Ten needles for acupuncture were pricked per time, and acupuncture was carried out for 28 times by 2/1. Average body temperature in 10 days was 36.09° C. with temperature difference and pulse difference maintained at −0.109° C. and 0.5 respectively. Temperature difference to the patient gradually turned to the negative by −0.21° C. following acupuncture, and symptoms at abdominal also witnessed a good turn. Furthermore, edema and other clinical symptoms also witnessed a favorable turn, and the face became florid. According to aforesaid theory, it can be judged that acupuncture is effective. Based on curative effect of 1-month therapy by the acupuncture doctor, symmetrical life information was tested for comparison. Average body temperature, temperature difference and pulse difference on that day were 36° C., 0.1° C. and 2 respectively, serving as a benchmark data. Ten needles for acupuncture by the doctor were pricked per time, and acupuncture was carried out for 28 times. Average body temperature in 10 days was 36.09° C., which increased by 0.09° C. as compared with benchmark data; temperature difference was −0.109° C., which increased by −0.21° C. as compared with benchmark data; pulse difference decreased as compared with previous one. Similarly, 1-month data was taken for symmetrical balance based quantified therapy. Average temperature from 3, 25-4 to 23 (see FIG. 11-13) was 36.2° C., which increased by 0.2° C. as compared with benchmark data; temperature difference was −0.01° C., which increased by −0.19 as compared with benchmark data; pulse difference was 0.2. As compared with acupuncture doctor, converted temperature difference was relatively low. However, it was stable in terms of assurance of balance of cardiovascular system and rise of body temperature. Moreover, due to cumulative effect, converted temperature difference was up to −0.15° C. following therapy for 2 months, which increased by 0.25° C. as compared with benchmark data or by −0.04° C. as compared with the renowned acupuncture doctor in Shenzhen. Average body temperature in 10 days was 36.59° C., which increased by 0.59° C. as compared with benchmark data. Pulse difference was 0, indicating the balance of cardiovascular system. In view of overall favorable turn to general conditions, we believe that the effect of symmetrical balance based quantitative therapy is comprehensive, stable and reliable.

### Example 6. Acupuncture is Effective for Treatment of Tumors as Proved by Symmetrical Data

To improve curative effect, acupuncture frequency was increased to 40 needles/time/day. Average body temperature, temperature difference and pulse difference in 10 days were 36.09° C., −0.109° C. and 1.5 respectively. Negative value of temperature difference to patient after acupuncture was −0.0° C., which decreased by 0.049° C. as compared with average value in previous 10 days; pulse difference increased by 1, which was less than previous one according to judgment based on aforesaid theory. The patient suffered from pain to left hip and legs, and such clinical symptoms as edema also become severe. This indicates that increased acupuncture frequency is unlikely to improve curative effect (see datasheet 1 for life information). Such clinical symptoms as pain to right hip and legs of the patient, edema and abdominal distention became gradually severe accompanied by loss of acupuncture efficacy. Meanwhile, general conditions also became worse. Accordingly, temperature difference gradually turned to positive, and reached +0.23° C. following acupuncture to the patient. 10-day average temperature difference changed from −0.064 at the completion of acupuncture to −0.013° C.-0.0091° C.

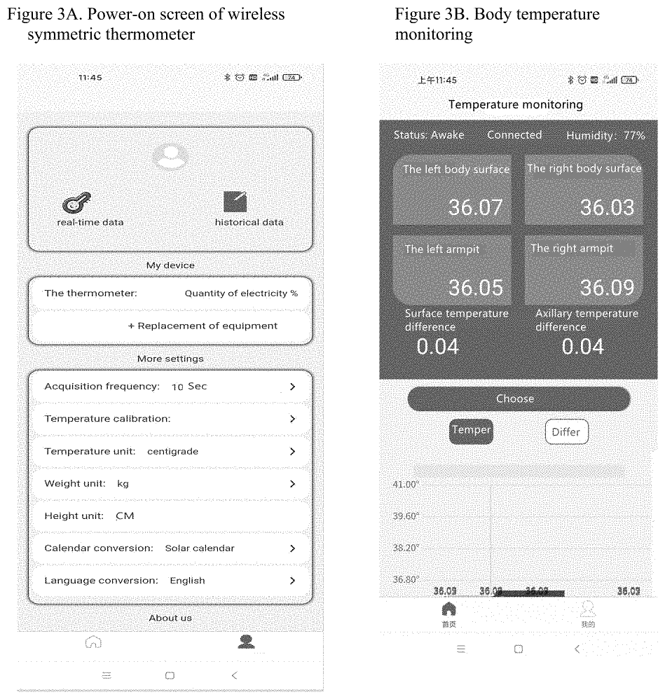

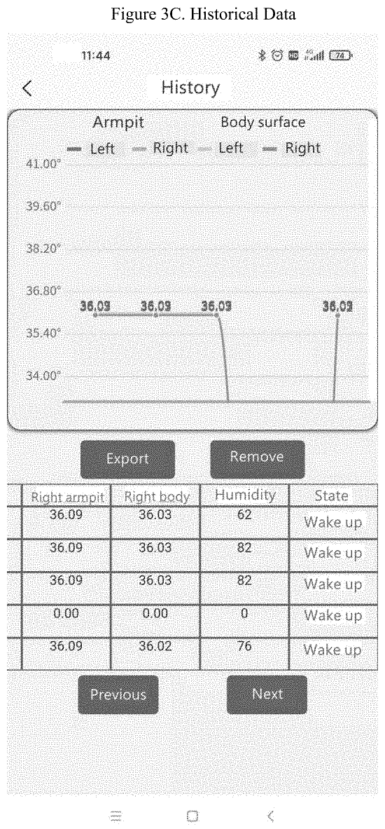

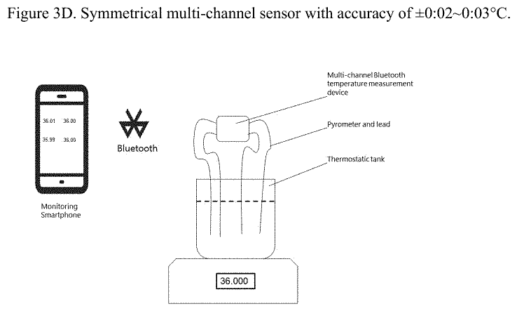

“Life monitor” serves as front life monitoring terminal of body & Environment sensor network (BESN), which is linked up with life quantification platform. By taking this theory as guide, we can realize setup of such environmental factors as comprehensive automatic regulation of temperature and humidity, electrolyte (including trace elements and various drugs), voice, color and image. We also take symmetrical balance-based quantification as guide for indoor design of pulley available for manual or auto regulations and “health space generator) for such sports approaches as dumbbell and pedal driving, and thereby take them as approaches for health intervention and therapy. (See FIG. 3A-3D).

### Example 7. Health Space Generator for Treatment of Colorectal Cancer

A patient suffering from colorectal cancer can make use of health generator for oriented control of body temperature, humidity, air circulation direction, size, sound, vision, color and other external stimuli. Furthermore, the patient can select such upper limb motions as pulling of pulley and lifting of dumbbell, and proceed with observation before and after therapy and rehabilitation as well as view of temperature difference measurement records. The patient received therapy for 51 days. The patient suffered from pain on 5/10 before treatment, and started to receive treatment of cool in the upper and heat in the lower by using “health space generator” on March 28. The infrared spectrum electric heating plate was used to regulate the temperature, the sauna steam generator was used to regulate the humidity, and the CD was used to play the rhythmic music. Indoor generator is in light yellow, of which upper and lower temperature was 9-21° C. and 48-52° C. respectively. The patient pulled two plastic lifting rings with both hands for sliding on the pulley. Taking the left hand as a reference, up and down motion for one time constitute a round of pull. Weight of each dumbbell is 1.51b. Lifting and back pulling with both hands constitute a round of pull. Frequency of pain before treatment was 5/10. Average value of life information at symmetrical parts in the first 10 days was 36.22, and average information difference was 0; frequency of pain was 1-1.5/10; temperature difference was −0.11. Average value of life information at symmetrical parts in the second 10 days was 36.21, and average information difference was −0.01; average frequency of pain was 1.3. Average value of life information at symmetrical parts in the third 10 days was 36.15, and average information difference was −0.09; average frequency of pain was 1.4. Average value of life information at symmetrical parts in the fourth 10 days was 36.2, and average information difference was −0.09; average frequency of pain was 1.4. Average value of life information at symmetrical parts in the fifth 10 days was 36.26, and average information difference was −0.11; average frequency of pain was 1.49. Average value of life information at symmetrical parts in the sixth 10 days was 36.58, and average information difference was −0.15; average frequency of pain was 1.51. Following treatment by “health space generator: for more than 50 days, frequency of pain suffered by the patient decreased from 5/10 to 1-1.5/10. Edema to the right leg was significantly alleviated; sleep witnessed a gradual improvement; appetite was enhanced, and white blood cells increased. General conditions and Immune mechanism were being improved. As to aforesaid “health space generator”, it has significant efficacy in treatment of advanced cancer that is the most difficult for human beings. The conception provided by such generator has been used for treatment of numerous patients suffering from advanced caners, and has achieved a satisfactory curative effect, not to mention other diseases. This is due to the fact that it is based on fundamental mechanism to ensure overall balance of human body. Moreover, it aims to provide solutions with natural or simulated natural methods, which has no side effect, and can significantly reduce health insurance while safeguarding health of human beings. (See Table 5).

TABLE 5
Health Space Generator of Cool in the Upper and Heat in the Lower for
Treatment of Intestinal Cancer

| | Pulley | | | | | | | | | |
|---|---|---|---|---|---|---|---|---|---|---|
| | pull | | | | Body temperature | | | Body temperature | | |
| | time | Dumb | Temperature | | before use | | | after use | | |
| Date | Min/ | bell | ° C. | | | | Dif- | | | Dif- |
| 2010 | time | Time | Up | Down | L | R | ference | L | R | ference |
| 25-Mar | | | | | | | | | | |
| 26-Mar | | | | | | | | | | |
| 27-Mar | | | | | | | | | | |
| 28-Mar | | | 48 | 18 | | | | | | |
| 29-Mar | | | 48 | 17 | | | | | | |
| 30-Mar | | | 48 | 19 | | | | | | |
| 31-Mar | | | 48 | 18 | | | | | | |
| 1-Apr | | | 48 | 18 | | | | | | |
| 2-Apr | | | 48 | 17 | | | | | | |
| 3-Apr | 25/1400 | 300 | 48 | 16 | 37.2 | 36.67 | 0.53 | 35.98 | 35.96 | 0.02 |
| 4-Apr | 25/1400 | 500 | 52 | 16 | 36.15 | 36.25 | −0.1 | 35.02 | 35.23 | −0.21 |
| 5-Apr | 25/1400 | 600 | 50 | 21 | 36.62 | 36.85 | −0.23 | 34.65 | 35.65 | −1 |
| 6-Apr | 25/1400 | 500 | 50 | 21 | 36.36 | 36.64 | −0.28 | 35.27 | 35.95 | −0.68 |
| 7-Apr | 25/1400 | 400 | 50 | 21 | 36.08 | 36.15 | −0.07 | 35.1 | 35.14 | −0.04 |
| 8-Apr | 25/1400 | 500 | 50 | 16 | 36.76 | 36.75 | 0.01 | 35.54 | 35.62 | −0.08 |
| 9-Apr | 25/1400 | 100 | 50 | 16 | 36.51 | 36.65 | −0.14 | 36.74 | 36.89 | −0.15 |
| 10-Apr | 25/1400 | 500 | 52 | 16 | 36.35 | 36.48 | −0.13 | 35.46 | 35.86 | −0.4 |
| 11-Apr | 25/1400 | 100 | 52 | 9 | 36.31 | 36.28 | 0.03 | 36.12 | 36.17 | −0.05 |
| 12-Apr | 20/1200 | 100 | 52 | 12 | 36.3 | 36.3 | 0 | 36.11 | 36.26 | −0.15 |
| 13-Apr | 20/1200 | 100 | 53 | 12 | 36.6 | 36.82 | −0.22 | 35.7 | 35.9 | −0.2 |
| 14-Apr | 20/1000 | 100 | 53 | 14 | 36.71 | 36.6 | 0.11 | 36.04 | 35.97 | 0.07 |
| 15-Apr | 20/1000 | 800 | 53 | 12 | 36.49 | 36.56 | −0.07 | 36.45 | 36.32 | 0.13 |
| 16-Apr | 20/1000 | 100 | 53 | 12 | 35.97 | 36.1 | −0.13 | 36.55 | 36.6 | −0.05 |
| 17-Apr | 20/1000 | 1000 | 57 | 12 | 36.22 | 36.37 | −0.15 | 34.85 | 35.32 | −0.47 |
| 18-Apr | 20/1000 | 1200 | 57 | 12 | 36.52 | 36.57 | −0.05 | 35.93 | 35.9 | 0.03 |
| 19-Apr | 15/800 | 1200 | 42 | 12 | 36.22 | 36.42 | −0.2 | 35.61 | 35.73 | −0.12 |
| 20-Apr | 15/800 | 1200 | 42 | 14 | 36.23 | 36.37 | −0.14 | 35.5 | 35.7 | −0.2 |
| 21-Apr | 15/800 | 1200 | 42 | 14 | 36.36 | 36.51 | −0.15 | 36.1 | 36.22 | −0.12 |
| 22-Apr | 15/800 | 1200 | 42 | 14 | 36.11 | 36.06 | 0.05 | 36.05 | 36.35 | −0.3 |
| 23- | 15/800 | 1200 | 42 | 12 | 36.54 | 36.67 | −0.13 | 36.24 | 36.29 | −0.05 |
| Apr | | | | | | | | | | |
| 24- | 15/800 | 1200 | 42 | 14 | 36.26 | 36.44 | −0.18 | 36.14 | 36.24 | −0.1 |
| Apr | | | | | | | | | | |
| 25- | 10/500 | 500 | 42 | 14 | 36.45 | 36.2 | 0.25 | 35.3 | 35.4 | −0.1 |
| Apr | | | | | | | | | | |
| 26- | 10/500 | 500 | 42 | 14 | 36.47 | 36.51 | −0.04 | 35.05 | 35.02 | 0.03 |
| Apr | | | | | | | | | | |
| 27- | 10/500 | 800 | 42 | 14 | 36.42 | 36.26 | 0.16 | 36.41 | 36.37 | 0.04 |
| Apr | | | | | | | | | | |
| 28- | None | None | None | None | 36.22 | 36.36 | −0.14 | None | None | |
| Apr | | | | | | | | | | |
| 29- | None | None | 42 | 14 | None | None | #VALUE | None | None | |
| Apr | | | | | | | | | | |
| 30- | None | None | None | None | 36.34 | 36.5 | −0.16 | 36.05 | 36.25 | −0.2 |
| Apr | | | | | | | | | | |
| 1- | 10/500 | 400 | 42 | 15 | 36.12 | 36.42 | −0.3 | 36.4 | 36.6 | −0.2 |
| May | | | | | | | | | | |
| 2- | 10/500 | 800 | 42 | 15 | 35.7 | 35.9 | −0.2 | 36.3 | 36.5 | −0.2 |
| May | | | | | | | | | | |
| 3- | 10/500 | 400 | 42 | 15 | 35.8 | 36 | −0.2 | 35.4 | 35.6 | −0.2 |
| May | | | | | | | | | | |
| 4- | 10/400 | 600 | None | None | None | None | #VALUE | None | None | #VALUE |
| May | | | | | | | | | | |
| 5- | 10/400 | 600 | 42 | 14 | 36.55 | 36.55 | 0 | 36.46 | 36.55 | −0.09 |
| May | | | | | | | | | | |
| 6- | 10/400 | 600 | 42 | 14 | 36.6 | 36.7 | −0.1 | 36.6 | 36.7 | −0.1 |
| May | | | | | | | | | | |
| 7- | 10/401 | 600 | 42 | 14 | 36.78 | 36.78 | 0 | 36.44 | 36.57 | −0.13 |
| May | | | | | | | | | | |
| 8-May | 10/400 | 800 | | | | | 0 | | | 0 |
| 9-May | 10/500 | 1000 | 42 | 14 | 37.05 | 37.1 | −0.05 | 36.95 | 37.15 | −0.2 |
| 10- | 10/500 | 1000 | 42 | 14 | 36.68 | 36.87 | −0.19 | 36.27 | 36.38 | −0.11 |
| May | | | | | | | | | | |
| 11- | 10/500 | 1000 | 42 | 14 | 35.54 | 36.82 | −1.28 | 36.34 | 36.68 | −0.34 |
| May | | | | | | | | | | |
| 12- | | | 42 | 13 | 36.7 | 36.77 | −0.07 | 36.36 | 36.5 | −0.14 |
| May | | | | | | | | | | |
| 13-May | None | | | | | | 0 | | | 0 |
| 14-May | None | | | | | | 0 | | | 0 |
| 15-May | 10/500 | 800 | 42 | 15 | 36.85 | 37.1 | −0.25 | 36.25 | 36.35 | −0.1 |
| 16- | 10/500 | 800 | 42 | 15 | 36.5 | 36.74 | −0.24 | 36.55 | 36.68 | −0.13 |
| May | | | | | | | | | | |
| 17- | 10/500 | 1000 | | | | | 0 | | | 0 |
| May | | | | | | | | | | |
| 18- | 10/500 | 800 | 42 | 16 | 36.74 | 37 | −0.26 | 36.54 | 36.68 | −0.14 |
| May | | | | | | | | | | |
| 19- | 10/500 | 1000 | | | | | | | | |
| May | | | | | | | | | | |
| 20-May | | | | | | | | | | |
| 21- | 10/1000 | 500 | | | | | | | | |
| May | | | | | | | | | | |
| 22-May | 10/500 | 800 | | | | | | | | |
| 23-May | 10/500 | 500 | | | | | | | | |
| 24-May | 10/500 | 500 | | | | | | | | |
| 25-May | 10/500 | 500 | | | | | | | | |

### Example 8. Individualized Object of Reference for Temporal and Spatial Impact

Impact of time and space as well as climate on health accounts for 7% of the total, which is in lack of comprehensive quantification. It is applicable to proceed with quantification of such natural environments as climate, geography, air temperature, humidity, wind and rain as well as location and orientation of people, advantages and disadvantages over health and their degrees.

Sky, earth, time and space are factor affecting individualization. Wherein, impact of the sun on weather and climate on the earth is the major aspect: 1) Thermal state of the earth and atmosphere serves as the basic factor for weather changes as well as underlying cause for variation to chills and fever of human body. Climatic energy on the earth is mainly from solar radiation. 2) Uneven distribution of solar radiation in radial direction may result in changes to global atmospheric circulation and climate. 3) Clouds in the sky blocks sunshine to make upper body temperature rise and negative value increase; thick clouds (including that before rain) may result in increase in negative value and decrease in positive value.

In an embodiment, diagnosis mode as stated in health intervention approaches for dynamic observation of physiological data according to present invention comprises use of Heavenly Stems data. 10 days constitute a Heavenly Stem (from the first day to the tenth day of the lunar calendar), representing internal organs of the body. It is applicable to precisely confirm the physique of a person based on 10-day basic body temperature and specific mathematical model.

### Example 9. Impact of the Moon on the Earth and Human Psychology

As proposed by ancient medical experts, rise and fall of Qi and blood in human body is corresponding to the moon. The cycle of waxing and waning of the full moon and new moon has tremendous impact on the growth and development of ecological things on the earth. For instance, on the 1^{st} day of lunar calendar, the moon moves to the position between the earth and the sun, and the illuminated hemisphere is just back to the earth. This is so called new moon. The moon covers 9.4 million km² solar radiation with its cross-sectional area to the extent of resulting in significant reduction of thermal radiation effect, relatively low land surface temperature and reduced temperature of Yongquan point of foot—intensified cool in the lower part of body (increase in positive value of heat in the upper part of the body accordingly). For this reason, patients of esophageal cancer feel more comfortable and have the lowest mortality accompanied by deteriorated conditions of patients of colorectal cancer. On the third or the fourth day of the lunar calendar, the moon moves from the position between the earth and the sun to take the form of crescent moon to the east of the sun. The half-moon illuminated by the sun is oriented towards the west, part of which faces the earth. As a result of it, the earth bears lunar reflection in addition to solar radiation. Meanwhile, thermal radiation from the earth increases, resulting in rise of sole temperature. On this account, increased negative value on the third or fourth day of the lunar calendar may alleviate the syndrome of heat in the upper and cool in the lower, and patients of colorectal cancer will feel relatively comfortable. On the contrary, the syndrome of cold in the upper and heat in the lower is to be intensified, resulting in increased negative value, progression of esophageal cancer and significant increase of mortality.

Just like the tide, menstruation of the female is also closely related to the moon. The menstrual period is more concentrated before and after the new moon period; whereas ovulation period is closer to the full moon period. The gravity of the moon may produce biological tide in human body. As human body is composed of 70% liquid, when the lunar gravity acts on the pineal gland, it will affect the “micro tide” to endocrine. According to similar theory as proposed by Professor Ross Maris, periodic waxing and waning of the moon has an impact on health and even behavior, indicating that blood vessels may rupture more easily than at other times, and the frequency of heart attacks may increase during the period of full and crescent moon.

Cold and heat are most important influential factor of environment on individualization; whereas celestial movement is the most important influential factor on temperature change. Different human species, races and ethnic groups as well as crowds of the same ethnic group in different regions manifest different adaptabilities to temperature, and there are great differences to the critical temperature of health fluctuation as incurred by temperature factors in different places. Impact of air temperature on human body is mainly represented by regulation of body temperature. Climatic factors, such as rainwater and humidity, are directly associated with individualization of symmetrical equilibrium” 1) After rainfall, water drops from the sky to the ground to make the lower part of the body cool. This is favorable for reduction of negative value and rise of positive value; 2) Rise of humidity and atmospheric temperature will make it difficult for human body dissipate heat to the extent of resulting in rise of body temperature; 3) this is harmful to patients with negative value, which may make the illness even worse.

Basis of precise diagnosis and targeted intervention: There are four seasons in a year, and the climate may be cold, hot, warm and cool; temperature difference has its orientation, and physique can be divided into coldness and warmness; motion-based heat dissipation is a vector; food can be cold, hot, warm and cool ones; emotion may be passionate and indifferent; such aspects are unified by cold and heat to set off health directionality. This will bring into being “uniform expression of physique, lifestyle and environmental information as well as a uniform intervention method”.

In an embodiment, the data acquisition device of health management and diagnosis system driven by information on individualized remote health management system according to the present invention further comprises a synchronous environment and close-in environmental information acquisition device that is used to measure and send local environment and close-in environmental information of the user to IOT platform when the symmetrical equilibrium acquisition device is used to measure physiological data. It is available for quantification of correlation to local/close-in environment and expression of symmetrical equilibrium trend of interrelation between differences to dynamic symmetrical physiological data with impact on health and information on physiological state corresponding to environmental information.

### Example 10. Health Digitalization is a Complicated Systematic Engineering Process

Precise and digitalized health management is the most complicated systematic engineering work. So many disciplines are to be integrated, so many differences and reference factors are to be considered, and precise test is to be implemented. Human beings are lazy and convenience dependent in nature. Most people are unwilling to use aforesaid precise and complicated programs. Furthermore, a new habit is established based on overcoming the old one. Any bad habit that is known to the public may still exist due to the law of inertia.

Only accompanied by accelerated development of IT industry and establishment of AI industry in the 21^{st} century, can aforesaid processes be realized based on obvious effect achieved by using sensor and AI for auto acquisition and real-time feedback of information via mobile APP. For this purpose, we have been working hard and persistently for 12 years.

Presently, health management is mainly based on advantages of IT industry to improve transmission speed of disease information, storage function, quality as well as pre-warning and real-time positioning of critical life information. It is also expected to realize remote sales of health products and prescription drugs in an effort to improve efficiency and service quality, shorten medical treatment time, create convenient conditions for patients, and control medical expenses for cost reduction. Information transmission is the common ground for both IT and medical industries, which represents the early stage of tele-medicine.

There exists a great gap between IT and the thought of medicine. Presently, any party can only understand regulatory thought of the other party, and is in lack of perceptual experiences on individualized performance, objects of reference for judgment and improvement of curative effect and parameters for prevention of medical errors. In view of response of clinical medicine to lifestyle, I have spent more than ten years to inquire into approaches for equipment to reflect the thought of medicine, and have incorporated abnormal behaviors and habits of patients into hardware and software. I have managed to transform digitalization of doctor's language into IT programs to make use of AI to realize integration of IT and the thought of medicine. This aims to solve the problem of “what can be done” by data acquisition, realize real-time health intervention, reflect symmetrical equilibrium, improve precision, and implement real-time monitoring of life activities for integration and standardization. This can either ensure accuracy of health state assessment and treatment of diseases or satisfy user's requirements for diversification. Realize digitalized survey of impact of individualized lifestyle and standardized health management on the organism, and change the mode of “calculation of average level” for most of people in existing medical structure, namely standardized medical mode and the mechanical mode of “elimination of diseases means health”. Use big data for health expression, and proceed with comprehensive assessment and forecast by referring to statistical requirements of NIH and FDA so as to ensure its dual significance of safety and efficiency in terms of medicine and statistics.

Symmetrical equilibrium medicine can be deemed as a criterion of curative effect for assurance of timely diagnosis and treatment. To establish a biomedical network and new disease classification system for symmetrical equilibrium medicine, it is necessary to classify diseases and physiques in addition to establishment of a dynamic data network integrating symmetrical information test with health and lifestyle records. Only in this way can we solve the problem with integration of individualization and standardization, and produce enormous economic benefits. 1. It is not limited to description of diseases, which directly aims at profound understanding and numerical description of mechanism, pathogenic mechanism and intervention approaches in combination with symmetrical information difference. 2. Symmetrical quantification technology is expected to eliminate aforesaid bottleneck in an attempt to change isolated treatment into collaborative medicine, acquire valuable data for analysis and decision making, and eventually provide individualized intervention propositions. 3. Provide lifelong health archive, and establish interrelation with physical examination indicators to provide quantitative basis for disease. 4. Market initiation by right of curative effect is beyond the reach of other health management enterprises—the basis for establishment of customer service system that aims to occupy the market in membership mode. 5. OEM takes processing of various food and sports supplies with registered trademark as a profit earning approach; aforesaid commodities subject to market orientation, individualized selection and quantification, which cannot be replaced by other commercial circles—the basis for long-term maintenance of market. 6. Individualized food is always based on the principle of small profit and quick turnover, which is characterized by high nutrition pertinence and relatively cheap price. 7. Integration of remote management, short-distance training and service—global face-to-face training, will be characterized by high diversification, large varieties and increased revenue accompanied by Rehabilitation and health care, tourism and health care services. 8. Establish an integrated channel for stroke emergency and health management; promote rehabilitation and health management based on miraculous effect in treatment of acute and severe diseases, and obtain the key for curative effect-based market initiation—expand family rehabilitation, and make use of miracle in treatment of known diseases to bring into being conception on treatment of unknown diseases. 9. Adhere to the market principle of cloud network and coverage expansion; take clinically acute and severe diseases and high-end customers as the start point; take membership and network as a whole as well as promotion platform; take the market as the basis. Use the minority to bring along the majority to occupy global market.

Take the nature of lifestyle and curative effect as a criterion for classification of big data based on aforesaid thought; this can ensure objectiveness of test while maintaining individualized characteristics. Sufferer's physiological parameters as collected remote medical monitoring system and such information as video, audio and images are to be sent in time to monitoring center via the communication network for dynamic tracking of pathogenic development and assurance of timely diagnosis and treatment.

The symmetrical equilibrium quantification platform according to the present invention is available for operation via smart phone APP. Measure armpit temperature, and transmit measurement results via the Bluetooth in low power consumption mode to realize continuous measurement of body temperature. It is applicable to make a judgment of physique and health state based on temperature on both sides of human body as well as temperature difference. For a person in good health, temperature difference is below ±0.2° C., and blood pressure difference on both sides is below 10 mmHg while pulse remain unchanged (accounting for 7-8% population). Sub-health is represented by the following facts: temperature difference exceeds ±0.2° C.; blood pressure difference is over 10 mmHg; pulse difference occurs for 1 time. Meanwhile, disease stage is represented by the following facts: temperature difference exceeds ±0.3-0.35° C.; pulse difference occurs for 2 times; blood pressure difference is over 20 mmHg. Where pulse difference occurs for 3 times, or temperature difference on both sides is over ±0.45° C., it means that life state becomes imbalanced, and disease becomes even worse. The more severe the disease is, the greater the temperature difference will be; when illness is alleviated, temperature difference is to become small; such change reflects functional variation and quantitative relationship. Unbalanced sub-health state measurement can be identified before the change of tissue morphology (except for 1-5% strong physique).

### Example 11. Provide Digitalized Objective Basis for Overall and Long-Term Health

Reference values directly related to anti-aging performance: For symmetrical equilibrium health management, we can proceed with tracking and management for 10 years to the maximum. It is discovered that it is against normal physiological rules, which serves as an anti-aging approach. Symmetrical equilibrium health monitoring management was carried out for XXX ten years ago. Basal body temperature during the first ten days is stated as follows: Temperature of L and R armpits was 35.73° C. and 35.53° C. respectively (average body temperature is 35.68° C.), and difference was +0.24° C. Health conditions were poor at that time, represented by Left carotid plaque, premature ventricular contraction and left ventricular high voltage. Symmetrical equilibrium health management was commenced accordingly. On Jun. 29, 2019, temperature of L and R armpits was 35.897° C. and 35.96° C. respectively (average body temperature was 35.93° C.), and difference was −0.06° C. As compared with those ten years before, body temperature increased by 0.25° C., and temperature difference decreased by −0.3 cm. Health state was significantly improved. The patient was energetic and vigorous, seemed to be 50 or 60 years old. Body weight was 58K, waist circumference was 82 points, and ECG was basically normal. In view of normal physiological conditions, temperature difference will become greater with increasing age. However, scientific individualized health management has broken the normal physiological conventions. This is exactly an anti-aging performance.

TABLE 6
Reference Values Directly Related to Anti-aging Performance

| | (Name) | Male | Age | 69 | |
|---|---|---|---|---|---|
| | Time | | Body | | |
| | | Time of | temperature° C. | | |
| | Date | temperature | L | R | |
| | M D Y | measurement | armpit | armpit | |
| | Data | Time | Ltemp | Rtemp | |
| 8-lung | 2019 June 20 | 6:00 | 36.4 | 35.94 | 0.46 |
| 9-bladder | 2019 June 21 | 4:45 | 36.31 | 35.93 | 0.38 |
| 10-kidney | 2019 June 22 | 7:35 | 36.37 | 36.45 | −0.08 |
| 1-liver | 2019 June 23 | 7:20 | 35.71 | 35.84 | −0.13 |
| 2- | 2019 June 24 | 6:30 | 35.68 | 35.57 | 0.11 |
| gallbladder | | | | | |
| 3-small | 2019 June 25 | 7:00 | 36.08 | 36.21 | −0.13 |
| intestine | | | | | |
| 4-heart | 2019 June 26 | 6:40 | 35.5 | 35.74 | −0.24 |
| 5-stomach | 2019 June 27 | 8:40 | 35.09 | 35.68 | −0.59 |
| 6-spleen | 2019 June 28 | 6:53 | 35.95 | 36.24 | −0.29 |
| 7-large | 2019 June 29 | 8:10 | 35.88 | 36 | −0.12 |
| intestine | | | | | |
| | | | 35.897 | 35.96 | −0.063 |

### Example 12. Unity of Equipment Precision, IT and Thought of Medicine Serves as the Basis of Precise Medicine. Impact of Moving Probe on Symmetrical Temperature

In an embodiment, it is discovered that left paster moves downward by 1-1.5 cm due to the motion of upper limbs. Therefore, the left paster is affixed again and shifted upwards to the armpit. When left paster probe moves downwards by 0.5 cm, and then the paster is revealed for upward movement, it is discovered that temperature on the left side of the body gradually rises; 3 minutes later, temperature on both sides becomes equal, and turns to positive value; Jiquan point at armpit is the center, which serves as a benchmark point.

| | 09-28 | Left | Right | Temperature |
|---|---|---|---|---|
| No. | Time: 09-28 | temperature | temperature | Difference |
| 108 | 19:52:05 | 36.42 | 36.63 | −0.21 |
| 107 | 19:51:03 | 36.42 | 36.60 | −0.18 |
| 106 | 09-28 19:49:59 | 36.40 | 36.57 | −0.17 |
| 105 | 09-28 19:48:56 | 36.37 | 36.53 | −0.16 |
| 104 | 09-28 19:47:53 | 36.33 | 36.53 | −0.20 |
| 103 | 09-28 19:46:51 | 36.48 | 36.52 | −0.04 |
| 102 | 09-28 19:45:47 | 36.48 | 36.52 | −0.04 |
| 101 | 09-28 19:44:44 | 36.47 | 36.52 | 0.05 |
| 100 | 09-28 19:43:42 | 36.46 | 36.53 | 0.07 |
| 99 | 09-28 19:42:39 | 36.44 | 36.52 | 0.08 |
| 98 | 09-28 19:41:35 | 36.42 | 36.47 | −0.05 |
| 97 | 09-28 19:40:32 | 36.41 | 36.56 | −0.15 |
| 96 | 09-28 19:39:29 | 36.39 | 36.54 | −0.15 |
| 95 | 09-28 19:38:26 | 36.37 | 36.52 | −0.15 |
| 94 | 09-28 19:37:24 | 36.33 | 36.51 | −0.18 |
| 93 | 09-28 19:36:20 | 36.45 | 36.48 | 0.03 |
| 92 | 09-28 19:35:17 | 36.44 | 36.44 | 0.00 |

In an embodiment, an electric fan is located to the left of the testee, which is about 1 m away from the left upper arm. The electric fan has reduced temperature on the left side. As discovered through repeated observation, our transducer is in series connection, of which both sides are on the same chip. Therefore, when air is blown from outside in an attempt to reduce left-side temperature, right-side temperature will compensatively rise. Consequently, right-side temperature will rise to 37.43° C. instead. Once the electric fan is turned off, left-side temperature will rise simultaneously with right-side temperature. On one hand, we can see impact of ambient temperature on body temperature of the testee; on the other hand, we can notice the chain effect produced by two temperature pasters using the same transducer.

In an embodiment, the electric fan is shifted to the right side that is 0.5 m away from right hand to reduce right-side temperature. When air is blown from outside in an attempt to reduce right-side temperature, right-side temperature will compensatively rise. Consequently, left-side temperature will rise to 37.43° C. instead.

As requested by APP, only left-side temperature and temperature difference (it will be positive value if left-side temperature is greater than right-side one, and vice versa) are to be displayed on the screen to save the space. Carry out processing on hardware to make sure that smartphone screen is corresponding to temperature measurement data at the time when different functional blocks are pressed. Furthermore, time and values of corresponding testing facilities are to be displayed on the platform. Left body temperature and temperature difference before and after pressing of functional block are to be transmitted to the platform for display. Left temperature and temperature difference after sports minus that before sport are to be transmitted to the platform for display. APP is to be in Chinese and English versions available for mutual conversion. Date of Gregorian calendar and the lunar calendar (the lunar calendar might be concealed on the smartphone, which can be displayed on the platform) is to be displayed simultaneously. 1-10^{th} day of the lunar calendar corresponds to the viscera. When the body temperature continues to rise for 20 minutes in the morning, the starting body temperature is deemed to be the basal body temperature. If it is possible to list it in together with current ambient temperature/humidity, and calculate the mean value in 10 consecutive days for display on the platform? When temperature difference on both sides of the user is over 0.35° C., or temperature difference on one side is below 35° C. or over 38° C., or pulse difference on both sides occurs for over 3 times, buzz alarm is to be given to remind the user to pay attention to the hazard incurred by current behavior/emotion on the health, and stop current behavior/emotion.

The cold and heat environment formed by air conditioning, heating installation and electric fans may change people's cold-heat environment to the extent of incurring significant impact on human health. For therapeutic effect on human beings, it is similar to physical heating and cooling. Comprehension of heating, cooling and sleeping posture is an important point for understanding of lifestyle. APP may provide a method that is most efficient for health according to relevant information.

### Example 13. Symmetrical Equilibrium Digitalization can Provide Instructions on Individualized Design of Medical Instruments

The data on which medical instruments, diagnosis and treatment methods and curative effect are based shall be closely related to microclimate environment. The macro-environment of local external climate is a general character, and microenvironment closed to each person is a specific character. As to individualization, it is essential to consider individualized difference to macro-environment and micro-environment closed to each person in addition to individualized difference to each person. In this way, it is applicable to precisely obtain individualized data on 7% action as imposed by climatic environment on human health.

Among wearable equipment for remote health management, close-in microenvironment has more direct impact on diagnosis, treatment methods and curative effect. Presently, equipment for regulation of various environmental conditions is becoming increasingly developed, for instance, temperature and humidity affecting the health. More importantly, it refers to indoor environment. After all, temperature and humidity inside clothes are different from external differences.

TABLE 7
Data on Real-time Monitoring of Microclimate Environment

| | L | R | Temperature |
|---|---|---|---|
| Date and time | temperature | temperature | difference |
| Aug. 23, 2020 12:11 | 36.39 | 36.75 | −0.36 |
| Aug. 23, 2020 12:11 | 36.36 | 36.68 | −0.32 |
| Aug. 23, 2020 12:11 | 36.35 | 36.47 | −0.12 |
| Aug. 23, 2020 12:10 | 36.36 | 36.88 | −0.52 |

New adhesive tape of transducer was affixed to the armpit again

| | | | |
|---|---|---|---|
| Aug. 23, 2020 12:10 | 36.35 | 37.07 | −0.72 |
| Aug. 23, 2020 12:10 | 36.35 | 37.05 | −0.7 |
| Aug. 23, 2020 12:09 | 36.37 | 37.04 | −0.67 |
| Aug. 23, 2020 12:09 | 36.35 | 37.04 | −0.69 |
| Aug. 23, 2020 12:09 | 36.33 | 37.11 | −0.78 |
| Aug. 23, 2020 12:08 | 36.3 | 37.1 | −0.8 |

When the transducer was unplugged, it was discovered that the adhesive
tape was wet, and the right side was wetter than the left side

| | | | |
|---|---|---|---|
| Aug. 23, 2020 12:08 | 36.28 | 37.08 | −0.8 |

Omron measurement: 36.16-36.46° C., 141/79.74-143/92.74.

| | | | |
|---|---|---|---|
| Aug. 23, 2020 12:08 | 36.23 | 37.03 | −0.8 |

According to examination by touching with both hands, the adhesive tape
became wet, resulting in increased conduction of sensor piece. See the
photo.

| | | | |
|---|---|---|---|
| Aug. 23, 2020 12:07 | 36.25 | 37.17 | −0.92 |

15 minutes later, temperature at right armpit increased by 1° C.
unexpectedly.

| | | | |
|---|---|---|---|
| Aug. 23, 2020 12:07 | 36.2 | 37.13 | −0.93 |
| Aug. 23, 2020 12:07 | 36.43 | 37.13 | −0.7 |
| Aug. 23, 2020 12:06 | 36.71 | 37.13 | −0.42 |
| Aug. 23, 2020 12:06 | 36.71 | 37.13 | −0.42 |
| Aug. 23, 2020 12:05 | 36.71 | 37.13 | −0.42 |
| Aug. 23, 2020 12:05 | 36.69 | 37.11 | −0.42 |
| Aug. 23, 2020 12:05 | 36.68 | 37.1 | −0.42 |
| Aug. 23, 2020 12:04 | 36.67 | 37.09 | −0.42 |
| Aug. 23, 2020 12:04 | 36.66 | 37.09 | −0.43 |
| Aug. 23, 2020 12:04 | 36.65 | 37.08 | −0.43 |
| Aug. 23, 2020 12:03 | 36.63 | 37.07 | −0.44 |
| Aug. 23, 2020 12:03 | 36.62 | 37.06 | −0.44 |
| Aug. 23, 2020 12:03 | 36.6 | 37.04 | −0.44 |
| Aug. 23, 2020 12:02 | 36.58 | 37.03 | −0.45 |
| Aug. 23, 2020 12:02 | 36.57 | 37.01 | −0.44 |
| Aug. 23, 2020 12:02 | 36.55 | 37.01 | −0.46 |
| Aug. 23, 2020 12:01 | 36.53 | 37 | −0.47 |
| Aug. 23, 2020 12:01 | 36.51 | 36.98 | −0.47 |
| Aug. 23, 2020 12:01 | 36.5 | 36.96 | −0.46 |
| Aug. 23, 2020 12:00 | 36.47 | 36.95 | −0.48 |
| Aug. 23, 2020 12:00 | 36.45 | 36.94 | −0.49 |
| Aug. 23, 2020 12:00 | 36.42 | 36.92 | −0.5 |
| Aug. 23, 2020 11:59 | 36.4 | 36.91 | −0.51 |
| Aug. 23, 2020 11:59 | 36.38 | 36.89 | −0.51 |
| Aug. 23, 2020 11:58 | 36.35 | 36.87 | −0.52 |
| Aug. 23, 2020 11:58 | 36.33 | 36.85 | −0.52 |
| Aug. 23, 2020 11:58 | 36.31 | 36.83 | −0.52 |
| Aug. 23, 2020 11:57 | 36.3 | 36.8 | −0.5 |
| Aug. 23, 2020 11:57 | 36.3 | 36.79 | −0.49 |
| Aug. 23, 2020 11:57 | 36.28 | 36.76 | −0.48 |
| Aug. 23, 2020 11:56 | 36.25 | 36.74 | −0.49 |
| Aug. 23, 2020 11:56 | 36.23 | 36.72 | −0.49 |
| Aug. 23, 2020 11:56 | 36.2 | 36.69 | −0.49 |
| Aug. 23, 2020 11:55 | 36.12 | 36.67 | −0.55 |
| Aug. 23, 2020 11:55 | 36.07 | 36.64 | −0.57 |
| Aug. 23, 2020 11:55 | 36.1 | 36.6 | −0.5 |
| Aug. 23, 2020 11:54 | 36.2 | 36.58 | −0.38 |
| Aug. 23, 2020 11:54 | 36.21 | 36.53 | −0.32 |
| Aug. 23, 2020 11:54 | 36.21 | 36.52 | −0.31 |
| Aug. 23, 2020 11:54 | 36.21 | 36.52 | −0.31 |
| Aug. 23, 2020 11:54 | 36.21 | 36.51 | −0.3 |
| Aug. 23, 2020 11:54 | 36.21 | 36.5 | −0.29 |
| Aug. 23, 2020 11:53 | 36.22 | 36.5 | −0.28 |
| Aug. 23, 2020 11:53 | 36.22 | 36.49 | −0.27 |
| Aug. 23, 2020 11:53 | 36.22 | 36.48 | −0.26 |
| Aug. 23, 2020 11:53 | 36.22 | 36.48 | −0.26 |
| Aug. 23, 2020 11:53 | 36.22 | 36.47 | −0.25 |
| Aug. 23, 2020 11:53 | 36.22 | 36.46 | −0.24 |
| Aug. 23, 2020 11:53 | 36.23 | 36.45 | −0.22 |
| Aug. 23, 2020 11:53 | 36.23 | 36.45 | −0.22 |
| Aug. 23, 2020 11:53 | 36.24 | 36.44 | −0.2 |
| Aug. 23, 2020 11:53 | 36.24 | 36.43 | −0.19 |
| Aug. 23, 2020 11:53 | 36.24 | 36.42 | −0.18 |
| Aug. 23, 2020 11:52 | 36.25 | 36.41 | −0.16 |
| Aug. 23, 2020 11:52 | 36.25 | 36.4 | −0.15 |
| Aug. 23, 2020 11:52 | 36.25 | 36.4 | −0.15 |
| Aug. 23, 2020 11:52 | 36.25 | 36.38 | −0.13 |
| Aug. 23, 2020 11:52 | 36.25 | 36.37 | −0.12 |
| Aug. 23, 2020 11:52 | 36.25 | 36.35 | −0.1 |
| Aug. 23, 2020 11:52 | 36.25 | 36.34 | −0.09 |
| Aug. 23, 2020 11:52 | 36.25 | 36.32 | −0.07 |
| Aug. 23, 2020 11:52 | 36.24 | 36.3 | −0.06 |
| Aug. 23, 2020 11:52 | 36.24 | 36.28 | −0.04 |
| Aug. 23, 2020 11:52 | 36.23 | 36.24 | −0.01 |

2 minutes later, external temperature started to rise while right-side
temperature was continuously rising

| | | | |
|---|---|---|---|
| Aug. 23, 2020 11:52 | 36.23 | 36.22 | 0.01 |
| Aug. 23, 2020 11:51 | 36.24 | 36.2 | 0.04 |
| Aug. 23, 2020 11:51 | 36.3 | 36.13 | 0.17 |
| Aug. 23, 2020 11:51 | 36.29 | 36.15 | 0.14 |
| Aug. 23, 2020 11:51 | 36.28 | 36.24 | 0.04 |
| Aug. 23, 2020 11:51 | 36.27 | 36.27 | 0 |
| Aug. 23, 2020 11:51 | 36.26 | 36.31 | −0.05 |
| Aug. 23, 2020 11:51 | 36.25 | 36.29 | −0.04 |

To measure the body temperature, and guard against impact from air
temperature, electric fan and air conditioner were turned off

| | | | |
|---|---|---|---|
| 2020 Aug. 23.1151 | 36.25 | 36.27 | −0.02 |

11:51 When getting up to work and measure the body temperature, it was discovered that the adhesive tape was wet after unplugging of the sensor. The right side was wetter than the left side; the horizontal bar referred to normal adhesive tape.

Measurement of close-in ambient temperature: Install close-in air temperature transducer to the transducer chip. As the transducer is hung on the chest, its temperature is just the temperature with clothes on as well as the ambient temperature directly affecting people's health. According to our test, the difference between it and armpit temperature is normally 2˜3° C. It is applicable to estimate turn-over during sleep, and adjust real-time monitoring data based on difference between close-in ambient temperature and armpit temperature.

As discovered by us, the body temperature on both sides of a person is different, and sweat amount on both sides is also varied. As to the patient with the syndrome of heat in the upper and cool in the lower, sweat amount at right armpit is relatively higher than that at the left armpit. Therefore, right armpit temperature may rise significantly. At 11:51, 2020 Aug. 23, to measure the body temperature, and eliminate influence from air temperature, the tester turned off the electric fan and air conditioner. Body temperature measured at that time was 36.25-36.29, and temperature difference was 0.04° C. 2 minutes later, external temperature started to rise accompanied by continuous rise of left-side temperature. 15 minutes later, armpit temperature on the right increased by 1° C. unexpectedly (74-12:07:43.36.25-37.17.); temperature difference was −0.92° C. According to examination by touching with both hands, the adhesive tape became wet, resulting in increased conduction of sensor piece. As a result of it, the temperature increased (76-12:08:26:36.28-37.08); temperature difference was −0.80° C.

The temperature measured with Omron thermometer was 36.16-36.46° C., which was lower than it. This was due to the fact that Omron thermometer was directly attached to the skin for measurement while the transducer was fixed below the adhesive tape. We unplugged the transducer and found that the adhesive tape was wet; meanwhile, humidity on the right was higher than that on the left. (see the photo attached); for this reason, the humidity was relatively high. Therefore, the body temperature was higher than that as measured with Omron thermometer (normally, body temperature measured with Omron thermometer is higher than it). As the thermometer was inserted deeply into the skin, it was only required to wipe off sweat as noticed during use. Despite of the fact that any sweat noticed during use of thermometer can be wiped off, Bluetooth thermometer for real-time monitoring was to be worn for several or even more than 10 hours. Consequently, sweating and other phenomena would frequently occur.

Under the same temperature, the higher the relatively humidity is, the more significant rise of apparent temperature will be. As sweating may inevitably affect precision of data acquisition, we propose to install humidity transducer chip into the temperature sensor piece. Based on AI calculation, it is applicable to find relationship between humidity and temperature, and temperature acquired is more in compliance with actual physiological state. It is applicable to design all medical instruments for remote health management based on such conception.

### Example 14. Symmetrical Equilibrium Digitalization can Significantly Improve Curative Effect

Normal temperature difference was below 0.2° C.; the syndrome was so obvious accompanied by increase of temperature difference—judgment of curative effect for improvement of references for curative effect. Following symmetrical equilibrium intervention, the syndrome was significantly alleviated, and temperature difference was reduced or reversed. Acute and severe stroke was characterized by asymmetry with temperature difference before intervention up to ±0.4° C.2° C. Following proper treatment, temperature difference reduced by ±0.4° C.-0.8° C. to form a new symmetry; the severe state was eliminated, and vice versa—effective approaches for assessment of degree of hazards and treatment measures. By taking this as a guide, curative effect can be improved by several times, and severe and acute state can be eliminated in half month. 80% patients may witness a good turn from bedridden state to walking—a breakthrough point to open the market. As to patients of chronic diseases deemed to be abnormal through clinical physical examination at hospital, physical examination center and nursing home, objective examination indicators may witness a significant improvement, or become normal through symmetrical equilibrium health intervention for 3-6 months; meanwhile, clinical syndromes will witness an obviously good turn to produce a sense of well-being again. Human body is a unified whole, and symmetrical equilibrium is available for comprehensive regulation and control of lifestyle. In addition to major syndromes, complications such as diabetes and renal failure will also witness an obviously good turn. According to more than 200 stroke cases treated in strict accordance with symmetrical equilibrium approach, no failed case was discovered. Provide compatible prescriptions on diet, health care products and equipment, exercise methods and residence based on dynamic evaluation of physical signs, physique, lifestyle and environmental data as well as integration of human nature and clinical practice. This aims to realize individualized precise health management.

### Example 15. Viewing Symmetrical Equilibrium Medicine Based on Clinical Cases

Symmetrical equilibrium medicine aims at scientific design and excavation of life information on symmetrical parts of human body, information difference and extension of parameters to realize organic integration with time-space acquisition and processing as well as digitalization. It is connected to U-Health system via transducer node to realize “all-round and full-time” monitoring of human body health and environmental conditions. Proceed with careful and precise classification of cold and heat physiques, and implement individualized health intervention through test of data presented. Any change in lifestyle, psychological activities, surrounding natural and social environment will result in changes to health. Through measurement of physique and symmetrical body temperature, pulse, blood pressure and changes to left-right differences before and after occurrence of one certain variation factor, symmetrical equilibrium medicine aims to make a precise judgment as to whether it is favorable or harmful based on orientation and value of differences so as to identify potential hazards to health and unhealthy living habits. This is the only method that is available for quantification of data on lifestyle, environment and intervention approaches and acceptance of test of practice worldwide. Provide different prescriptions for diet, exercise method, drinking water temperature, water volume, dress, sleep and residence according to specific conditions based on biological mechanism and data on cold and heat physiques as well as nature of human beings and clinical practice. This aims to form a diagnosis and targeted intervention scheme, and proceed with individualized health intervention. Lay emphasis on provision of different pertinent lifestyles and treatment approaches for different people. Such diagnosis is somewhat related to disease name of Western medicine, which can serve as auxiliary diagnosis of diseases in terms of Western medicine—despite of the fact that specificity is inferior to Western medicine, its results can constitute a targeted intervention scheme, which can be directly used for clinical practice and health care. Therefore, it is more in compliance with objective existence of human body, which can accelerate feedback. Two completely different treatment schemes for treatment of the same disease are presented through cold-heat quantification of existing diseases—It has discovered underlying causes as to why the curative ratio of modern western medicine cannot exceed 36% in treatment of various chronic diseases. It has also found a way out for cancer, diabetes, stroke sequelae, weight reduction and other modern medical difficulties as well as a way for realization of harmony between human beings and nature. As limited by specific conditions, most of medical records collected are incomplete and unavailable for contrast with medium and large samples as well as statistical processing; it is only applicable to use the following method for statement by taking cancer and diabetes for instances. furthermore, horizontal comparison is to be replaced by vertical one: 1. Try to take diseases deemed to be incurable by modern medicine for instances; in this way, existing cases for treatment of such diseases will form a contrast group objectively to facilitate the reviewer to think in a rational manner; 2. Use vertical comparison to replace horizontal one: a. Symmetrical equilibrium medicine is not taken as the guide for treatment, which is only used for information acquisition and test. b. As to the same patient, symmetrical equilibrium medicine is used for information acquisition and test to provide a guidance on treatment for contrast with a. c. Normally, it is applicable to prove validity of control conditions based on different between treatment of symmetrical equilibrium medicine and that of “health space generator”. 3. Symmetrical equilibrium medicine was once used for treatment of cases of colorectal cancer; accompanied by life maintenance and good turn of disease, values gradually turned to negative; this has manifested the originality and orderliness of diagnosis and treatment in terms of symmetrical equilibrium medicine. 4. Proceed with classification of various diseases in terms of “positive” and “negative” (namely cold-heat attributes), and compare individual cases of illness state and symmetrical life information data before and after treatment to seek a common rule for outcome data. 5. Seek a common rule for valid outcome data in view of different disease spectrums, such lifestyles as recovery and sports as well as such treatment approaches as drug and chemotherapy. 6. Sports, diet, psychological equilibrium as well as drugs and health care facilities may result in changes to body temperature; however, left-right variation may be in different directions. Any of such directions determines the direction of treatment and diagnosis. This is the orientation of symmetrical equilibrium medicine.

In an embodiment, a rectal cancer patient, female, 39 years old, height: 156 cm, weight: 43 kg. Description of medical history: In December, 2007, hematochezia occurred to the patient without any inducement; as indicated by enteroscopy and CT, there was a 2×2 cm lump at 4-5 o'clock position of rectum that is 3 cm away from the anus; such lump was characterized by slightly rigid by pressing, rough surface, ulceration and bleeding; it was determined as rectal cancer through diagnosis. The 1^{st} chemotherapy was carried out on 2008 Jan. 4; local resection of rectal cancer+ileostomy at trans-sacral was carried out on 2008 Jan. 22. A 3×3 cm lump was discovered during operation, belonging to ulcerative type with unclear boundary. Pathological diagnosis: rectal adenocarcinoma, highly or moderately differentiated; cancer cells infiltrated into the superficial muscular layer, part of which infiltrated to medium and deep muscular layer. Immunohistochemistry (IHC): cerb (+), CEA (+), VEGF (−), assay: cea15, wbc4.81. The patient frequently suffered from abdominal pain in the past three and half years, and received chemotherapy for 6 times. In view of emotional entanglement with his spouse, state of disease became worse in the past one year, accompanied by transfer to lymph node. In recent four months, the right thigh has become swollen accompanied by dull pain (5-6/10) that may become worse when lying flat; it was also accompanied by cramp and spasm to right calf. Assay: CEA, 68. Assay on December 30: wbc2.0 (4.0-10.); Rbc4.12; albumin: 13.7 (11.0-16.0) GRAN1.2 (2.0-7.8); hemoglobin content: 33.2 (26-32); mononuclear: 3(4-13); other indicators are normal. From then on, the patient has suffered from pain to both lower limbs, especially on the left side; The pain from the sole of the foot to the outside of the leg as well as anterior superior spine in front of left bone of buttock is so obvious; the frequency is 5-7/10 approximately; such pain is persistent; abdominal discomfort or distension often result in paroxysmal colic and intestinal obstruction. At the end of 2010, his spouse contacted me and asked me to go to Shenzhen for formal symmetrical equilibrium treatment for him.

Dynamic monitoring data: 1. Benchmark data: Conditions& symmetrical life information before symmetrical equilibrium treatment shall serve as the reference for treatment in the future. When I arrived in Shenzhen on Mar. 25, 2010, the patient suffered from pain to right hip, knees and right thigh at a frequency of 5-6/10; the patient was unable to sleep at night accompanied by such syndromes as loose stool and swelling to right lower limbs. Measurement indicators are indicated as follows:

TABLE 8
Measurement Indications

| | | L | R | | | | | | |
|---|---|---|---|---|---|---|---|---|---|
| | Time of | temperature | temperature | Temperature | | | L | | Pulse |
| | measurement | ° C. | ° C. | difference | L BP | R BP | pulse | R | difference |
| 25- | 7:45 | 36.38 | 36.28 | 0.10 | 95/62 | 98/63 | 65 | 64 | 1 |
| Mar | | | | | | | | | |
| 26- | 9:20 | 36.38 | 36.28 | 0.10 | 99/58 | 99/60 | 66 | 61 | 5 |
| Mar | | | | | | | | | |

Viewing from Table 8, we can see benchmark data at the commencement of treatment: Average body temperature is 36° C.; temperature difference is 0.10; pulse difference has occurred for 3 times; average blood pressure: average systolic blood pressure on the left is 97 with average diastolic pressure up to 60; average systolic blood pressure on the right is 98 with average diastolic pressure up to 61.5; average left-right systolic blood pressure difference is −1, and average left-right diastolic pressure difference is −0.15.

### Example 16. Heavenly Stems Data

Heavenly Stems data: 10 days constitute a Heavenly Stem (from the first day to the tenth day of the lunar calendar), representing internal organs of the body. It is applicable to precisely confirm the physique of a person based on 10-day basic body temperature and specific mathematical model. Orientation and values for increase or decrease of information difference serves as an objective criterion for health intervention. Following symmetrical equilibrium treatment from March 25 to April 3 and from May 14 to May 23, Heavenly Stems temperature difference changed as follows: 0, −0.01, −0.09, −0.11 and −0.15° C. It can be seen clearly that values are gradually turning to negative accompanied by good turn of illness; therefore, such diagnosis has its originality.

TABLE 9
Average Body Temperature in 10 Days

| | Body | | | | | | | | | | | | |
|---|---|---|---|---|---|---|---|---|---|---|---|---|---|
| | tem- | | | | | | | | | | | | |
| | perature | ° C. | | | | | | | | | | | |
| | L | R | | | | | | | | | | | |
| | armpit | armpit | | | | | | | | L-P | L | | Pulse |
| Date | ° C. | ° C. | | L-P | | | R-P | | | difference | pulse | R pulse | difference |
| 25-Mar | 36.21 | 36.21 | −0.00 | 100 | 65 | 35 | 101 | 64.7 | 36.7 | −1.7 | 63.9 | 62.4 | 1.5 |
| to 3-Apr | | | | | | | | | | | | | |
| 4-Apr to | 36.20 | 36.21 | −0.01 | 104 | 71 | 33 | 104 | 73.5 | 30.9 | −2.2 | 64.3 | 64.3 | 0 |
| 13-Apr | | | | | | | | | | | | | |
| 14-Apr | 36.13 | 36.22 | −0.09 | 98.7 | 69 | 30 | 99.8 | 67.1 | 32.6 | | 63.4 | 63.2 | 0.2 |
| to 23- | | | | | | | | | | | | | |
| Apr | | | | | | | | | | | | | |
| 24-Apr | 36.15 | 36.24 | −0.09 | 93.9 | 63 | 31 | 95.3 | 64.4 | 30.9 | | 68.11 | 68.44 | 0.33 |
| to 3- | | | | | | | | | | | | | |
| May | | | | | | | | | | | | | |
| 4-May | 36.21 | 36.32 | −0.11 | 100 | 67 | 33 | 100 | 67.9 | 33 | | 70.9 | 70.7 | 0.2 |
| to 13- | | | | | | | | | | | | | |
| May | | | | | | | | | | | | | |
| 14-May | 36.52 | 36.67 | −0.15 | 100 | 68 | 32 | 100 | 66.6 | 33.4 | | 70.111 | 70.111 | 0 |
| to 23- | | | | | | | | | | | | | |
| May | | | | | | | | | | | | | |
| 24-May | 36.52 | 36.84 | −0.32 | 98 | 64 | 34 | 97 | 66 | 31 | | 66 | 66 | 0 |
| 25-May | 36.55 | 36.62 | −0.07 | 100 | 71 | 29 | 99 | 63 | 36 | | 62 | 64 | −2 |

Viewing from Table 9, it can be seen clearly that average body temperature in 10 days has gradually decreased from 0.1° C. on March 25 to −0.15 on 5/14-23; meanwhile, the state of disease has also witnessed a good turn accordingly. It is proved that cancer treatment and recovery process is in compliance with principle for symmetrical equilibrium quantification. By 14-25/5, left pulse was 70, exactly the same as right pulse. Average blood pressure in 10 days: average systolic pressure on the left was 100 with average diastolic pressure up to 68; average systolic pressure on the right was 100 with average diastolic pressure up to 66.6; average left-right systolic pressure difference was 0, and average left-right diastolic pressure difference was 1.4. Following treatment, average systolic pressure on the left and right became fully identical (balanced); average left-right diastolic pressure was 1.4. Furthermore, the patient had a healthy appetite in addition to slightly increase in body weight and good turn of sleep. Leukocyte index in hemogram also witnessed a rise.

Basic system data: the sympathetic and parasympathetic nerves are balanced when wake is regained, which features in minimum interference to symmetrical life information and the highest accuracy. It is applicable to accurately confirm the physique of a person based on basal body temperature in 10 days, symmetrical life information and specific mathematic model. Orientation and values for increase or decrease of information difference serves as an objective criterion for health intervention. Major or qualitative changes to physical conditions or state of an illness reflect correlation or causality based on temperature difference. Based on orientation and value of differences among them, it is applicable to make a precise judgment as to whether such life style, environmental factor, psychological activities and intervention approaches are favorable or harmful to you so as to identify potential hazards to health and unhealthy living habits. Table 10 shows that body temperature on both sides of the patient, pulse and blood pressure are also correlated. Thus, it can be seen that symmetrical equilibrium quantification is efficient in monitoring and treatment of cancers.

### Example 17. Life Information Data Collection

Life information data (see Table 10): According to the case of rectal cancer, the value changed from 0.1° C. on March 25 to −0.15° C. in the period from May 14 to May 23. The state of disease also witnessed a good turn.

TABLE 10
Life Information Data

| | L | R | | | | | | | | | | | | |
|---|---|---|---|---|---|---|---|---|---|---|---|---|---|---|
| | armpit | armpit | Temperature | | | | | | | | | L | R | |
| Date | ° C. | ° C. | difference | L-P | | | R-P | | | | | pulse | pulse | |
| 25- | 36.38 | 36.28 | 0.10 | 95 | 62 | 33 | 98 | 63 | 35 | | | 65 | 64 | 1 |
| Mar | | | | | | | | | | | | | | |
| 26- | 36.38 | 36.28 | 0.10 | 99 | 58 | 41 | 99 | 60 | 39 | | | 66 | 61 | 5 |
| Mar | | | | | | | | | | | | | | |
| 27- | 36.13 | 36.20 | −0.07 | 97 | 59 | 38 | 98 | 58 | 40 | | | 59 | 58 | 1 |
| Mar | | | | | | | | | | | | | | |
| 28- | 36.16 | 36.20 | −0.04 | 99 | 66 | 33 | 99 | 65 | 34 | | | 66 | 65 | 1 |
| Mar | | | | | | | | | | | | | | |
| 29- | 35.90 | 36.00 | −0.10 | 98 | 65 | 33 | 95 | 63 | 32 | | | 63 | 65 | −2 |
| Mar | | | | | | | | | | | | | | |
| 30- | 35.90 | 36.10 | −0.20 | 104 | 66 | 38 | 105 | 70 | 35 | | | 65 | 64 | 1 |
| Mar | | | | | | | | | | | | | | |
| 31- | 36.25 | 36.25 | 0.00 | 104 | 69 | 35 | 108 | 68 | 40 | | | 67 | 61 | 6 |
| Mar | | | | | | | | | | | | | | |
| 1- | 36.42 | 36.25 | 0.17 | 97 | 67 | 30 | 102 | 63 | 39 | | | 65 | 61 | 4 |
| Apr | | | | | | | | | | | | | | |
| 2- | 36.22 | 36.22 | 0.00 | 102 | 69 | 33 | 104 | 70 | 34 | | | 62 | 62 | 0 |
| Apr | | | | | | | | | | | | | | |
| 3- | 36.40 | 36.38 | 0.02 | 109 | 70 | 39 | 106 | 67 | 39 | | | 61 | 63 | −2 |
| Apr | | | | | | | | | | | | | | |
| | 36.21 | 36.22 | −0.00 | 100 | 65 | 35 | 101 | 64.7 | 36.7 | −1.7 | 0.4 | 63.9 | 62.4 | 1.5 |
| 4- | 36.37 | 36.11 | 0.26 | 104 | 68 | 36 | 104 | 74 | 30 | | | 64 | 63 | 1 |
| Apr | | | | | | | | | | | | | | |
| 5- | 35.90 | 36.01 | −0.11 | 109 | 76 | 33 | 109 | 77 | 32 | | | 69 | 68 | 1 |
| Apr | | | | | | | | | | | | | | |
| 6- | 36.21 | 36.22 | −0.01 | 111 | 73 | 38 | 104 | 76 | 28 | | | 63 | 65 | −2 |
| Apr | | | | | | | | | | | | | | |
| 7- | 36.30 | 36.30 | 0.00 | 101 | 71 | 30 | 100 | 71 | 29 | | | 62 | 61 | 1 |
| Apr | | | | | | | | | | | | | | |
| 8- | 36.42 | 36.35 | 0.07 | 108 | 74 | 34 | 102 | 78 | 24 | | | 66 | 65 | 1 |
| Apr | | | | | | | | | | | | | | |
| 9- | 35.93 | 35.85 | 0.08 | 104 | 74 | 30 | 107 | 70 | 37 | | | 62 | 63 | −1 |
| Apr | | | | | | | | | | | | | | |
| 10- | 36.38 | 36.45 | −0.07 | 101 | 62 | 39 | 102 | 68 | 34 | | | 63 | 63 | 0 |
| Apr | | | | | | | | | | | | | | |
| 11- | 36.20 | 36.10 | 0.10 | 102 | 70 | 32 | 104 | 69 | 35 | | | 66 | 66 | 0 |
| Apr | | | | | | | | | | | | | | |
| 12- | 35.99 | 36.25 | −0.26 | 103 | 71 | 32 | 106 | 76 | 30 | | | 60 | 60 | 0 |
| Apr | | | | | | | | | | | | | | |
| 13- | 36.28 | 36.45 | −0.17 | 101 | 74 | 27 | 106 | 76 | 30 | | | 68 | 69 | −1 |
| Apr | | | | | | | | | | | | | | |
| | 36.20 | 36.21 | −0.01 | 104 | 71 | 33 | 104 | 73.5 | 30.9 | −2.2 | | 64.3 | 64.3 | 0 |
| 14- | 36.28 | 36.32 | −0.04 | 102 | 71 | 31 | 105 | 69 | 36 | | | 60 | 60 | 0 |
| Apr | | | | | | | | | | | | | | |
| 15- | 36.20 | 36.21 | −0.01 | 101 | 72 | 29 | 101 | 70 | 31 | | | 60 | 60 | 0 |
| Apr | | | | | | | | | | | | | | |
| 16- | 36.06 | 36.18 | −0.12 | 112 | 79 | 33 | 107 | 73 | 34 | | | 60 | 60 | 0 |
| Apr | | | | | | | | | | | | | | |
| 17- | 36.32 | 36.36 | −0.04 | 97 | 68 | 29 | 101 | 70 | 31 | | | 64 | 64 | 0 |
| Apr | | | | | | | | | | | | | | |
| 18- | 36.38 | 36.43 | −0.05 | 101 | 67 | 34 | 103 | 69 | 34 | | | 62 | 62 | 0 |
| Apr | | | | | | | | | | | | | | |
| 19- | 35.66 | 35.94 | −0.28 | 95 | 68 | 27 | 96 | 64 | 32 | | | 64 | 64 | 0 |
| Apr | | | | | | | | | | | | | | |
| 20- | 35.98 | 36.1 | −0.12 | 95 | 66 | 29 | 98 | 66 | 32 | | | 60 | 60 | 0 |
| Apr | | | | | | | | | | | | | | |
| 21- | 36.17 | 36.22 | −0.05 | 97 | 69 | 28 | 99 | 67 | 32 | | | 66 | 66 | 0 |
| Apr | | | | | | | | | | | | | | |
| 22- | 36.04 | 36.17 | −0.13 | 91 | 64 | 27 | 98 | 60 | 38 | | | 67 | 66 | 1 |
| Apr | | | | | | | | | | | | | | |
| 23- | 36.17 | 36.24 | −0.07 | 96 | 66 | 30 | 96 | 68 | 28 | | | 71 | 70 | 1 |
| Apr | | | | | | | | | | | | | | |
| | 36.13 | 36.22 | −0.09 | 98.7 | 69 | 30 | 99.8 | 67.1 | 32.6 | | | 63.4 | 63.2 | 0.2 |
| 24- | 35.96 | 36.18 | −0.22 | 92 | 61 | 31 | 92 | 59 | 33 | | | 73 | 72 | 1 |
| Apr | | | | | | | | | | | | | | |
| 25- | 36.05 | 36.17 | −0.12 | 94 | 65 | 29 | 94 | 65 | 29 | | | 70 | 69 | 1 |
| Apr | | | | | | | | | | | | | | |
| 26- | 36.30 | 36.30 | 0.00 | 95 | 65 | 30 | 96 | 65 | 31 | | | 69 | 67 | 2 |
| Apr | | | | | | | | | | | | | | |
| 27- | 35.8 | 35.92 | −0.12 | 99 | 70 | 29 | 94 | 65 | 29 | | | 69 | 70 | −1 |
| Apr | | | | | | | | | | | | | | |
| 28- | 36.12 | 36.28 | −0.16 | 92 | 62 | 30 | 99 | 68 | 31 | | | 61 | 61 | 0 |
| Apr | | | | | | | | | | | | | | |
| 29- | 36.7 | 36.65 | 0.05 | | | | | | | | | | | |
| Apr | | | | | | | | | | | | | | |
| 30- | 36.6 | 36.5 | 0.10 | 96 | 57 | 39 | 96 | 61 | 35 | | | 60 | 61 | −1 |
| Apr | | | | | | | | | | | | | | |
| 1- | 36.54 | 36.73 | −0.19 | 86 | 58 | 28 | 94 | 65 | 29 | | | 67 | 68 | −1 |
| May | | | | | | | | | | | | | | |
| 2- | 35.1 | 35.3 | −0.20 | 96 | 65 | 31 | 98 | 69 | 29 | | | 73 | 75 | −2 |
| May | | | | | | | | | | | | | | |
| 3- | 36.29 | 36.33 | −0.04 | 95 | 64 | 31 | 95 | 60 | 35 | | | 71 | 73 | −2 |
| May | | | | | | | | | | | | | | |
| | 36.15 | 36.24 | −0.09 | 93.9 | 63 | 31 | 95.3 | 64.4 | 30.9 | | | 68.1 | 68.4 | −0.33 |
| 4- | 36.1 | 36.3 | −0.20 | 103 | 67 | 36 | 104 | 78 | 26 | | | 71 | 73 | −2 |
| May | | | | | | | | | | | | | | |
| 5- | 36.36 | 36.5 | −0.14 | 97 | 71 | 26 | 94 | 64 | 30 | | | 68 | 66 | 2 |
| May | | | | | | | | | | | | | | |
| 6- | 36.74 | 36.9 | −0.16 | 113 | 81 | 32 | 115 | 78 | 37 | | | 78 | 77 | 1 |
| May | | | | | | | | | | | | | | |
| 7- | 36.12 | 36.23 | −0.11 | 103 | 72 | 31 | 104 | 78 | 26 | | | 72 | 72 | 0 |
| May | | | | | | | | | | | | | | |
| 8- | 35.98 | 36.09 | −0.11 | 103 | 69 | 34 | 105 | 73 | 32 | | | | 75 | 0 |
| May | | | | | | | | | | | | | | |
| 9- | 36.2 | 36.28 | −0.08 | 97 | 54 | 43 | 92 | 61 | 31 | | | 72 | 74 | −2 |
| May | | | | | | | | | | | | | | |
| 10- | 36.27 | 36.32 | −0.05 | 106 | 70 | 36 | 99 | 67 | 32 | | | 63 | 63 | 0 |
| May | | | | | | | | | | | | | | |
| 11- | 36.27 | 36.27 | 0.00 | 90 | 63 | 27 | 93 | 59 | 34 | | | 72 | 73 | −1 |
| May | | | | | | | | | | | | | | |
| 12- | 36.1 | 36.17 | −0.07 | 97 | 64 | 33 | 97 | 61 | 36 | | | 71 | 69 | 2 |
| May | | | | | | | | | | | | | | |
| 13- | 35.97 | 36.12 | −0.15 | 93 | 62 | 31 | 99 | 60 | 39 | | | 67 | 65 | 2 |
| May | | | | | | | | | | | | | | |
| | 36.211 | 36.32 | −0.11 | 100 | 67 | 33 | 100 | 67.9 | 33 | | | 70.9 | 70.7 | 0.2 |
| 14- | 36.83 | 36.96 | −0.13 | 100 | 71 | 29 | 99 | 67 | 32 | | | 70 | 68 | 2 |
| May | | | | | | | | | | | | | | |
| 15- | 36.46 | 36.59 | −0.13 | 98 | 65 | 33 | 97 | 58 | 39 | | | 60 | 60 | 0 |
| May | | | | | | | | | | | | | | |
| 16- | 36.21 | 36.48 | −0.27 | 98 | 65 | 33 | 97 | 58 | 39 | | | 60 | 60 | 0 |
| May | | | | | | | | | | | | | | |
| 17- | 36.33 | 36.44 | −0.11 | 99 | 62 | 37 | 93 | 64 | 29 | | | 69 | 70 | −1 |
| May | | | | | | | | | | | | | | |
| 18- | 36.04 | 36.35 | −0.31 | | | | | | | | | | | |
| May | | | | | | | | | | | | | | |
| 19- | 36.48 | 36.6 | −0.12 | 94 | 65 | 29 | 97 | 64 | 33 | | | 80 | 80 | 0 |
| May | | | | | | | | | | | | | | |
| 20- | 36.68 | 36.88 | −0.20 | 99 | 68 | 31 | 100 | 69 | 31 | | | 78 | 80 | −2 |
| May | | | | | | | | | | | | | | |
| 21- | 36.36 | 36.4 | −0.04 | 106 | 74 | 32 | 110 | 67 | 43 | | | 67 | 67 | 0 |
| May | | | | | | | | | | | | | | |
| 22- | 37.12 | 37.27 | −0.15 | 100 | 71 | 29 | 100 | 74 | 26 | | | 67 | 68 | −1 |
| May | | | | | | | | | | | | | | |
| 23- | 36.69 | 36.69 | 0.00 | 106 | 75 | 3 | 110 | 78 | 32 | | | 80 | 78 | 2 |
| May | | | | | | | | | | | | | | |
| | 36.52 | 36.66 | −0.15 | 100 | 68 | 32 | 100 | 66.6 | 33.4 | | | 70.1 | 70.1 | 0 |
| 24- | 36.52 | 36.84 | −0.32 | 98 | 64 | 34 | 97 | 66 | 31 | | | 66 | 66 | 0 |
| May | | | | | | | | | | | | | | |
| 25- | 36.55 | 36.62 | −0.07 | 100 | 71 | 29 | 99 | 63 | 36 | | | 62 | 64 | −2 |
| May | | | | | | | | | | | | | | |

In the same way, the symmetrical balance collection device was used for measurement, and then the data of one month from March 25 to April 23 were taken (see the figures below). The average body temperature in ten days was 36.2° C., which was 0.2° C. higher than the benchmark data; the temperature difference was −0.01° C., which was −0.19 higher than the benchmark data; and the pulse difference was 0.2, and compared with the famous acupuncturist, the speed of temperature change was lower, but it was more stable for the balance of cardiovascular system and the rise of body temperature. Besides, Cumulative effects were observed. After 2 months of treatment, the temperature change reached −0.15° C., and compared with the benchmark data, the negative value was increased by −0.25° C. and −0.04° C. higher than that of the famous acupuncturist. The average body temperature in ten days was 36.59° C., which increased by 0.59° C. compared with the benchmark data; and the pulse difference was 0, indicating that the cardiovascular system was balanced. The overall situation has improved. Therefore, we believe that the symmetrical balance treatment effect is comprehensive, stable and reliable

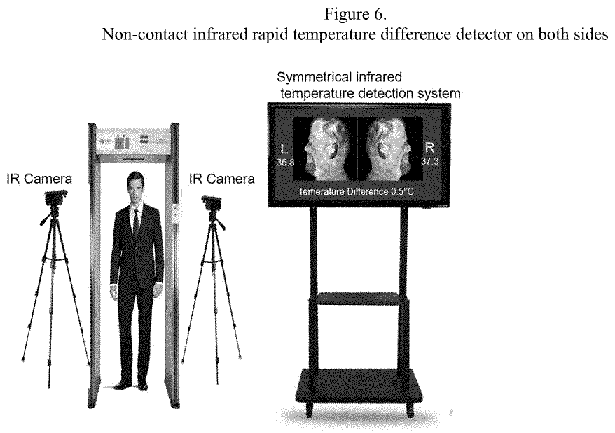

The relationship between maximum temperature difference to the patient in the first 10 days and organs corresponding to Heavenly Stems of lunar calendar can be used for auxiliary diagnosis and monitoring. The maximum value of the patient in the first 10 days is 0.17° C., which occurred on the seventh day of lunar calendar; Qigeng is corresponding to large intestine, which is in compliance with diagnosis of colorectal cancer. Accompanied by progress of treatment, negative value gradually decreased/turned to negative on the 7th, 17th and 271 day of each month (see the block), indicating that functions of large intestine were in the process of recovery. By May 20, 2010 (April 7 of the lunar calendar), temperature difference has reached −0.2° C.; meanwhile, functions of large intestine were witnessing a significant recovery (see Table 11 and FIG. 6).

TABLE 11
Life Information Data

| | L | R | Difference | | | | | | | L | R | |
|---|---|---|---|---|---|---|---|---|---|---|---|---|
| Date | armpit° C. | armpit° C. | ° C. | L-P | | Difference | R-P | | Difference | pulse | pulse | Difference |
| 1-Apr | 36.42 | 36.25 | 0.17 | 97 | 67 | 30 | 102 | 63 | 39 | 65 | 61 | 4 |
| 11-Apr | 36.20 | 36.10 | 0.10 | 102 | 70 | 32 | 104 | 69 | 35 | 66 | 66 | 0 |
| 20-Apr | 35.98 | 36.1 | −0.12 | 95 | 66 | 29 | 98 | 66 | 32 | 60 | 60 | 0 |
| 30-Apr | 36.6 | 36.5 | 0.10 | 96 | 57 | 39 | 96 | 61 | 35 | 60 | 61 | −1 |
| 10-May | 36.27 | 36.32 | −0.05 | 106 | 70 | 36 | 99 | 67 | 32 | 63 | 63 | 0 |
| 20-May | 36.68 | 36.88 | −0.20 | 99 | 68 | 31 | 100 | 69 | 31 | 78 | 80 | −2 |

### Example 18. Test Result on Patients in Shengzhen

With regard to diagnosis and disease monitoring of the patients in Shenzhen, body temperature on both sides, pulse and indoor temperature/humidity (° C.) were measured every 1-3 times according to quantitative medical theory for registration and calculation (see the table attached). 2. Adjustment to treatment and health intervention scheme based on calculations: A: drinking cool or cooled water with temperature below 20° C.; B: relevant exercises for the physique that is heat in the upper and cool in the lower, such as pulley pulling and dumbbell lifting; C: Eat a diet that raises (turns negative) the body temperature on the right side; D: Silk, wool and linen clothes other than cotton ones; E: sleeping with head towards the southeast; F: 1 h cold-heat equilibrium treatment via health generator at 11.30 a-12.30 p every day; G: Dialectical treatment and acupuncture for more than 20 times and administration of Chinese herbal medicine for more than 30 dosages. 3. We measured bilateral T/P/BP as well as difference value of left and right T/P/BP before and after the above health intervention behavior. This has overcome the abstract of “equilibrium” and the sense of separation and isolation of morphological medicine. It has manifested relevant reflection of objectivity, relevance, temporal and spatial dynamics, personalization, digitization and standardization of differences, which is available for comprehensive quantification of human body health trend, lifestyle and recovery orientation. 4. Curative effect after treatment: A: the pain suffered by the patient was alleviated following two-day treatment; B: temperature difference started to turn negative (it turned to −0.07 and −0.04 in a direction that is favorable for recovery); C: pulse difference on both sides became balanced; D: treatment of the syndrome of cool in the upper and heat in the lower was commenced on March 28 with the help of health generator; upper temperature was 9-21° C., and lower temperature was 48-57° C.; E: frequency of pain changed to 3/10 from April 2, which further changed to 1-1.5/10 from April 5; F: temperature difference was −0.11° C.; G: as to Chinese herbal medicine for promotion of blood circulation and removal of blood stasis, four blood stasis indicators observed, namely MCV, RDW-cv, MPV and PDW was in a trend of decrease from 3/24; meanwhile, tongue ecchymosis also witnessed a good turn. By the end of May, the pain (lasted for 2 months) had been controlled at 1-1.5/10 basically, and temperature difference had been maintained at −0.1-0.15 basically; the stool had become normal basically.

### Example 19. Treatment of a Caucasian Male with Advanced Colorectal Cancer

A Caucasian male suffering from advanced colorectal cancer was tested and treated. The patient has suffered from sigmoid cancer for five and half years with the state of disease worsened for two years. In 2005, the patient was diagnosed as sigmoid colon cancer; in July, 2009, the patient was admitted to hospital for abdominal pain and distension; recurrent sigmoid mass adjacent to the pelvis was found; such mass was 9×6 cm in size, which was in a process of transfer to lung and liver cancer. Meanwhile, colonic obstruction was discovered in addition to the left-side tumor resulting in obstructive nephropathy; for this reason, stent was installed to prevent left-side tumor from pressing against the left kidney. The patient received Colon-abdominal fistula to divert stool from the right lower abdomen. After operation, the patient suffered from severe edema to both lower limbs accompanied by sharp pain and spasm to both legs. The patient was inconvenient in walking, and became staggered. The patient once received chemotherapy, but failed. The patient had to use narcotic drugs to control the pain, and became addicted for this reason. The patient had such syndromes as sweating and chill. According to laboratory test, CFA was 36. As indicated by X-ray photos, cancer cells were transferring to the spinal cord, resulting in numbness to both legs. The patient sometimes loses consciousness and balance accompanied by numbness to right middle toe. Since late April, the patient had suffered pain, numbness and swelling to the left leg and stiffness to calf muscle—lymphedema. Diagnosis: Sigmoid colon cancer—stage IV sigmoid colon cancer. 2. Ureteral edema, hydronephrosis to left renal pelvis. As determined by a cancer hospital in the United States, the patient has only three-month survival time, and is requested to make a dying will. Following life quantification-based treatment for six and half months, the patient witnessed an obvious good turn in mental state. Body weight of the patient increased by 5 lbs accompanied by normal appetite and defecation. However, there was still severe pain to the left lower limb; the size of colorectal cancer Liver and kidney metastases and tumors were reduced by 30%; there was still a small amount of metastasis in the spinal cord, CEA16. Left-right temperature difference changed from positive one with left side greater than right side to the negative one with right side greater than left side. The following photo shows this process in more than half year. Accompanied by good turn of disease, changes to body temperature, pulse and blood pressure and their relationship are also developing in a trend that is in compliance with the rule of life quantification technology. the symmetrical equilibrium quantification has been Monitoring and treatment have been implemented for more than 300 days, and altogether more than 240 pairs of life information at symmetrical parts have been obtained. For data, please refer to Table 12. As to 10-day average value for life information on symmetrical parts, 10-day average body temperature and 10-day average body temperature difference have been changed from 36.55 and 0.095 to 36.72 and −0.14 respectively accompanied by the treatment. The left pulse is 86.86 and the right pulse is 86.71, becoming basically approximate. 10-day average body temperature is rising; 10-day average body temperature difference is turning to negative by 0.235; the patient has a good appetite accompanied by slight increase in body weight. (see Table 12). With the treatment, the life information about the symmetrical positions was as follows: the ten day average body temperature increased from 36.55° C. to 36.72° C., and the ten day average body temperature difference decreased from 0.095° C. to −0.14° C. Temperature and 10-day average body temperature difference have been changed from 36.55 to 36.72° C. at the beginning; the ten-day average body temperature difference ranges from 0.095 to −0.14° C. at the beginning The left pulse is 86.86 and the right pulse is 86.71, becoming basically approximate. 10-day average body temperature is rising; 10-day average body temperature difference is turning to negative by 0.235; the patient has a good appetite accompanied by slight increase in body weight. (see Table 12).

TABLE 12
Treatment of Advanced Colorectal Cancer in the First 10 Days

| | | | | | | Blood | | | |
|---|---|---|---|---|---|---|---|---|---|
| | | | | P-time/ | | pressure | | | |
| | Temperature ° C. | | | min | | (mmHg) | | | |
| Date | L | R | Difference | L | R | L | R | Sleep | Emotion |
| 29-Aug | 36.87 | 37.08 | −0.21 | | | | | | |
| 30=Aug | 36.8 | 36.95 | −0.15 | | | | | 6 h | crying |
| 31-Aug | 36.85 | 36.7 | 0.15 | | | | | 4 | sad |
| 1-Sep | 36.4 | 36.1 | 0.3 | | | | | | |
| 1-Sep | 36.6 | 36.4 | 0.2 | | | | | | hopeful |
| 2-Sep | 36.6 | 36.45 | 0.15 | | | | | 5 | hopeful |
| 3-Sep | 36.15 | 36.1 | 0.05 | 68 | | 98/80 | | | tired sad |
| 14-Sep | 36.75 | 36.8 | −0.05 | 85 | | 94/78 | | | very tired |
| 5-Sep | 35.9 | 36.15 | −0.25 | 75 | | 126/85 | | 8 | feel better |
| 17-Sep | 36.9 | 36.4 | 0.5 | 87 | | 150/93 | | | |
| 6-Sep | 36.9 | 36.4 | 0.5 | 80 | | | | | |
| 7-Sep | 36.4 | 36.45 | −0.05 | 91 | | 135/82 | | | |
| | 36.59 | 36.5 | 0.095 | | | | | | |

### Example 20. Treatment of a Colorectal Cancer Patient

The colorectal cancer is in positive value, which gradually turns to negative accompanied by life maintenance and good turn of state of disease following treatment by means of symmetrical equilibrium quantitative therapy. This has manifested the originality of symmetrical equilibrium quantitative diagnosis and treatment.

According to calculation based on data in the first 10 days before confirmation of physique of patients of colorectal cancer, it is discovered that the date corresponding to the maximum positive value is February 17 of lunar calendar for the female patient (4,1-large intestine, 0.17° C.). and July 17 of lunar calendar for the male patient. As to the two patients, the organ corresponding to Heavenly Stems of traditional Chinese medicine is just the large intestine, indicating troubles to the large intestine.

Measure variations to symmetrical body temperature, pulse and blood pressure as related to a certain syndrome before and after treatment to realize digitalized survey of curative effect. In an embodiment, a woman has a heat physique characterized by cold in the upper and heat in the lower. She is allergic to climate, and has suffered from nasal obstruction and rhinocnesmus for a week. She once received acupuncture therapy at my clinic. It is applicable to monitor treatment of allergy by means of acupuncture through test of life information at symmetrical parts. Reduction in two-side life information difference is correlated to curative effect. In an embodiment, a man has a cold physique characterized by heat in the upper and cold in the lower. Two months ago, Low back pain caused by handling injury, he started to suffer a lumbago with intensity up to 5-8/10; the state of illness is varied, sometimes good, and sometimes bad, which may become even worse in case of sports and bending. Diagnosis result is stated as follows: L4-5 and L5-S1 broad annulus fibrosis and bulging. Bilateral neural foraminal stenosis. Subligamentous herniated discs at both levels. Annular tears are present at these 2-disc levels. It is applicable to monitor treatment of pains by means of acupuncture based on test of life information at symmetrical parts. Two-side life information difference increases in direction that is opposite to that of the physique (for instance, the patient has a physique of positive value); After treatment, a negative difference occurred, and the increase of the negative difference was positively correlated with the curative effect. In an embodiment, a man has a cold physique characterized by heat in the upper and cold in the lower. He has been beset with restricted motion of neck, right shoulder and upper arm as well as motion obstruction for 2 months. Two-side life information difference increases in a direction that is opposite to that of the physique (for instance, the patient has a cold physique of positive value). Body temperature difference has resulted in negative difference following treatment, of which increase is positively correlated with curative effect. Pulse difference has been changed from 6 times/minute at the beginning to once/minute. Accordingly, syndrome has also disappeared. (see Table 13).

TABLE 13
Treatment of Neck, Right Shoulder and Upper Arm

| Date | | T- | T-Right | | | | | |
|---|---|---|---|---|---|---|---|---|
| 2010 | Time | Left ° C. | ° C. | ° C. | L-P | R-p | Difference | effect |
| 6-22 | 10:15a | 36.81 | 36.6 | 0.21 | 96 | 90 | 6 | Degree of restriction |
| | | | | | | | | before acupuncture is up |
| | | | | | | | | to 5/10 |
| | 11:30a | 36.57 | 36.42 | 0.15 | 92 | 89 | 3 | Degree of restriction after |
| | | | | | | | | acupuncture is 2/10 |
| 6-23 | 7:30a | 36.2 | 35.97 | 0.23 | 95 | 91 | 4 | Degree of restriction |
| | | | | | | | | before 2^{nd} acupuncture is |
| | | | | | | | | 2/10 |
| | 8:23a | 37.31 | 37.4 | 0.1 | 86 | 82 | 4 | Degree of restriction after |
| | | | | | | | | acupuncture is 1-2/10 |
| | 9:00a | 36.61 | 36.69 | 0.08 | 81 | 78 | 3 | Shoulder fully alleviated, |
| | | | | | | | | neck: 1-2/10 |
| 6-24 | 6:14p | 36.67 | 36.45 | −0.05 | 76 | 73 | 3 | Shoulder fully alleviated, |
| | | | | | | | | neck: 1-2/10 |
| | 7:00p | 36.61 | 36.67 | −0.06 | 75 | 74 | 1 | Completely alleviated |

### Example 21. Treating a Man has a Cold Physique Characterized by Heat in the Upper Body and Cold in the Lower Body

In an embodiment, a man has a cold physique characterized by heat in the upper and cold in the lower. 2 months ago, he suddenly had a pain to right shoulder that was unavailable for movement at that time; furthermore, he could not turn his head. He once received treatment at hospital for 3 times, but failed. Therefore, he came here for help. (see Table 14).

TABLE 14
Treatment of Pain to Right Shoulder

| Date | | T- | T- | | | | |
|---|---|---|---|---|---|---|---|
| 2009 | Time | Left ° C. | Right ° C. | | L-P | R-p | effect |
| 30- | 2.26p | 36.8 | 36.78 | 0.02 | | | Pain at diagnosis: |
| Sep | | | | | | | 8/10 |
| | 2.52p | 36.67 | 36.66 | 0.01 | | | Pain alleviated after |
| | | | | | | | acupuncture |
| | 3.38p | 36.45 | 36.69 | −0.24 | | | Pain after 30-minute |
| | | | | | | | indwelling: 3/10 |

### Example 22. Test of Curative Effect

It aims at assessment and quantification of living environment and impact of drugs on human health. In an embodiment, a man has a physique characterized by heat in the upper and cold in the lower. Following exposure to the sun in the outdoor open space (air temperature is 37° C., and ground temperature is 49° C.), he felt gum swelling and pain to the right upper posterior opsigenes; he also felt an approximately 5/10 pain to gum of right upper tooth when licking with tongue. According to measurement of body temperature at both armpits and pulse of both wrists (see the following table), temperature difference was up to 0.5° C. 10 minutes later, two-side pulse difference was up to 4, indicating serious imbalance. Toothache was persistent, and became severe gradually. 4 hours later, he felt 8/10 pain to gum of right upper tooth when licking with tongue; meanwhile, gum was swelling++—indicating that heat in the lower was expanding from the bottom to the top. At that time, the patient got a fever with average value of body temperature up to 37.36; however, temperature difference and pulse difference decreased to −0.09 and 2 times respectively. Despite of the fact that syndrome was getting worse, symmetrical life information difference was witnessing a good turn, indicating that body immunity was being enhanced. For this reason, conditions will be better after recovery. I requested the patient to take Cepsule capsules by 0.25 g×3 (Cephalexin) before rest. On the 20th day, the patient waked up and felt obvious alleviation of gum pain; the degree of pain to the gum of the right upper tooth when licking with tongue was 2/10 approximately. The patient took Cepsule capsules by 0.25 g×2. Tid; temperature difference and pulse difference were −0.18 and 2 respectively. On the 21^{st} day, the patient waked up, and found that gum swelling and pain disappeared; he only felt an approximately 0-1/10 pain to gum of the right upper tooth when licking with the tongue. The patient then took Cepsule capsules by 0.25 g×2. Tid; temperature difference were up to −0.04; blood pressure variation rule was uniform, indicating that this drug was effective. (see Table 15).

TABLE 15
Test of Curative Effect

| Date | | L arm | R arm | Temperature | L | R | Pulse | BP | BP | BP |
|---|---|---|---|---|---|---|---|---|---|---|
| 2010 | Time | T ° C. | T ° C. | difference ° C. | pulse | pulse | difference | L | R | R |
| 19-Jul | 3:43 p | 36.71 | 36.79 | −0.08 | 92 | 89 | 3 | | | |
| | 4:5 p | 36.9 | 36.4 | 0.5 | 102 | 100 | 2 | | | |
| | 5:04 p | 37.17 | 36.77 | 0.4 | 100 | 104 | −4 | | | |
| | 8:53 p | 37.31 | 37.4 | −0.09 | 102 | 100 | 2 | | | |
| 19-Jul | 7:43 a | 36.3 | 36.27 | 0.03 | 86 | 86 | 0 | | | |
| | 12:32 p | | | | | | | 85 | 139 | 86 |
| | | | | | | | | 92 | 3 | 149 |
| | | 36.02 | 36.24 | −0.22 | 89 | 86 | 3 | 139 | 125 | 99 |
| | 2:26 p | 36.03 | 36.28 | −0.25 | 98 | 95 | 3 | 93 | 131 | 85 |
| | 2:31 | | | | 95 | 89 | 6 | 90 | 138 | 99 |
| | 2:26 p | 36.08 | 36.48 | −0.4 | 92 | 94 | −2 | 100 | 133 | 86 |
| | 3:39 p | | | | | | | 85 | 125 | 91 |
| | | | | | | | | 94 | −1 | 141 |
| 19-Jul | 3:43 p | 36.71 | 36.79 | −0.08 | 92 | 89 | 3 | 13 | 143 | 104 |
| | | | | | | | | 7 | | |
| | 4:52 p | 36.9 | 36.4 | 0.5 | 102 | 100 | 2 | 87 | 127 | 87 |
| | 5:04 p | 37.17 | 36.77 | 0.4 | 100 | 104 | −4 | 85 | 142 | 85 |
| | 6:53 p | 37.31 | 37.4 | −0.09 | 102 | 100 | 2 | 84 | 135 | 100 |
| | 7:53 p | 36.64 | 36.4 | 0.24 | 78 | 88 | −10 | 95 | 143 | 102 |
| | 8:53 p | 37.31 | 37.4 | −0.09 | 102 | 100 | 2 | | | |
| 20-Jul | 5:39 a | 36.08 | 36.26 | −0.18 | 82 | 80 | 2 | 80 | 125 | 79 |
| 21-Jul | 7.23 a | 36 | 36.04 | −0.04 | 82 | 79 | 3 | 128 | | |
| | 10:25 a | 36.14 | 36.09 | 0.05 | 95 | 94 | 1 | 79 | 119 | 79 |
| | 3:13 p | 36.4 | 36.5 | −0.1 | 81 | 84 | −3 | 75 | 122 | 80 |

### Example 23. Symmetrical Balance Method Aims to Classify Various Diseases of Western Medicine into Cold and Heat

Symmetrical balance aims to integrate the pathological process of various diseases in Western medicine. Observe its dynamics and development trends through the changing trend of cold and heat, which may cause Western medicine to adopt completely opposite treatment plans for the same disease—It has discovered underlying causes as to why the curative ratio of modern western medicine cannot exceed 36% in treatment of various chronic diseases. The following paragraphs take diabetes as an instance for statement. Diabetes is defined as follows in Western medicine: Diabetes mellitus (DM, DiabetesMellitus) the word is to describe a multi-cause of metabolic disease characterized by chronic high blood sugar, accompanied by insulin (Insulin) secretion and/or the role of defects caused by sugar, fat and protein metabolism. Therefore, any diabetic patient is treated based on the guiding principle of high blood glucose and insufficient Insulin secretion. Take insulin, the hormone that highs blood sugar, as the foundation of treatment. In fact, insulin is only a bioactive substance, which can produce an effect on human body. However, different physiques may result in different or even completely opposite physiological effect. Symmetrical equilibrium quantification is expected to divide diabetic patients into heat and cold physiques through testing. For the same diabetes, completely different treatment approaches are to be taken in view of different physiques. Diabetes of cold physique: The result of abnormal heat metabolism may result in heat distribution characterized by cold in the upper and heat in the lower. Therefore, in the treatment of Dafa, the guidelines of reducing the calories of the lower body and increasing the calories of the upper body are adopted everywhere and all the time, in order to cooperate with the treatment of drugs. Furthermore, various physiochemical approaches are used to promote this process. a. drink hot water other than cold one—Drinking hot water is favorable for increase of heat in the upper body and promotion of thermodynamic equilibrium. b. Wear thicker for the upper body and less for the lower body. c. Only do or do more exercises and motions to lower body to ensure heat dissipation from lower body; do less or do no exercises or motions to upper body to facilitate reduction of heat dissipation. d. Take various external cooling measures to lower body, such as ice bag, such cooling devices as freezer, cool air, cold water and so on to facilitate heat dissipation from lower body; as to upper body, various external heating approaches are used to ensure heat dissipation from upper body, such as various electric heating devices, hot water, heating installation and so on. e. In external environment (including region as well as selection of design of residence), it is applicable to facilitate heat dissipation from lower body and increase of heat in upper body through design and intervention as possible. f. As to diets, it is recommended to eat meats, fruits, vegetables, beverages and fats that are favorable for increase of heat in upper body and heat dissipation in lower body. g. As to selection of Chinese traditional herbal medicines and western drugs, it is recommended to take Chinese traditional herbal medicines and western drugs that favorable for increase of heat in upper body and heat dissipation in lower body. However, it is just opposite for diabetic patients of cold physique.

### Example 24. Type I Diabetes Patients

Type I diabetes of cold in the upper and heat in the lower: It is a male patient born in 1969 with height and weight up to 1.72 m and 60.5 k (11.5 k lower than the standard weight). He was diagnosed by hospital as diabetes at the age of 25 in view of excessive drinking, urination and food, obvious weight loss and Type 1 diabetes. by laboratory test. Before using insulin (10 u×2), he had been taking hypoglycemic drugs for 2 years (without any effect). 8 years ago, aforesaid symptoms became even more worse, and the dosage of insulin was increased to 20 u×2 after he was diagnosed by hospital as type I diabetes. The patient has a family history of diabetes. On May 17, 2009, he came to seek my medical advice. The assay results on that day are stated as follows: glucose 197, AIC 9.8%, T-ch 4.04 mmol and TG 0.56 mmol; insulin 10 u Bid in, and METFORMIN HYDROCHLORIDE TABLETS (0.5 g Bid) were used. 10-day average symmetrical life information difference-temperature difference during the period from May 17, 2009 to May 26, 2009 was −0.18. Thus, the heat physique of cold in the upper and heat in the lower was confirmed. Therefore, I implemented treatment to the patient with aforesaid methods, and adjusted his sleep orientation—head oriented towards the southeast with adequate sunshine and feet oriented towards the northwest that is well ventilated. According to measurement of two walls, temperature difference was up to 1° C. From May 28, the patient kept walking for 2-3 hours per day. Meanwhile, he selected to step into the ice bucket for cooling and drink 50-60° C. hot water to maintain thermodynamic equilibrium. The patient used Insulin (10.u) before breakfast and dinner per day for 14 days, commencing from May 16. No other drugs were used. From the 12th day, it was changed to injection (10.u) before breakfast; such injection was stopped before dinner. Despite of this, the patient witnessed a reduction in blood glucose, from 197 on May 17 to 121 on June 10 and 81 on June 11. Viewing from the following table, it can be seen that following additional reversed temperature regulation of 1-3 hours per time or squatting or walking for 1-3 hours per day, temperature was increasing towards positive difference. There exists obvious correlation between the two. (See Table 16)

TABLE 16
Diabetes of Heat physique (Cold in the Upper and Heat in the Lower)

| | L | R | | | | | | | | |
|---|---|---|---|---|---|---|---|---|---|---|
| | armpit | armpit | | | | | | | | |
| | ° C. | ° C. | | L | R | | L arm | L arm | R arm | R arm |
| 05-17 | 36.2 | 36.5 | −0.3 | | | | | | | |
| 05-18 | 36 | 36.3 | −0.3 | | | | | | | |
| 05-19 | 36.2 | 36.4 | −0.2 | | | | | | | |
| 05-20 | 36.2 | 36.4 | −0.2 | | | | | | | |
| 05-21 | 35.9 | 35.9 | 0 | | | | | | | |
| 05-22 | 36.2 | 36.4 | −0.2 | | | | | | | |
| 05-23 | 36.2 | 36.3 | −0.1 | | | | | | | |
| 05-24 | 36.1 | 36.3 | −0.2 | | | | | | | |
| 05-25 | 36.7 | 36.8 | −0.1 | | | | | | | |
| 05-26 | 36.8 | 37 | −0.2 | | | | | | | |
| | 36.25 | 36.43 | −0.2 | | | | | | | |
| 05-27 | 36.37 | 36.64 | −0.3 | | | | | | | |
| 05-28 | 36.03 | 35.96 | 0.07 | 56 | 60 | −4 | 100 | 65 | 110 | 60 |
| | 35.96 | 36.89 | −0.9 | | | 0 | 120 | 70 | 115 | 65 |
| | 36.56 | 36.65 | −0.1 | | | 0 | | | | |
| | 36.24 | 36.22 | 0.02 | | | 0 | | | | |
| | 36.05 | 36.1 | −0.1 | | | 0 | | | | |
| 05-29 | 36.75 | 36.77 | −0 | | | 0 | 100 | 75 | 95 | 70 |
| | 36.32 | 36.38 | −0.1 | 37 | 60 | −23 | 102 | 72 | 106 | 68 |
| 05-30 | 36.16 | 36.24 | −0.1 | 52 | 55 | −3 | 100 | 60 | 100 | 55 |
| | 36.42 | 36.59 | −0.2 | | | 0 | | | | |
| | 36.56 | 36.55 | 0.01 | | | 0 | | | | |
| | 36.47 | 36.52 | −0.1 | | | 0 | | | | |
| | 36.12 | 36.26 | −0.1 | | | 0 | | | | |
| | 36.29 | 36.34 | −0.1 | | | 0 | | | | |
| | 36.32 | 36.34 | −0 | | | 0 | | | | |
| | 36.72 | 36.6 | 0.12 | | | 0 | | | | |
| | 36.29 | 36.1 | 0.19 | | | 0 | | | | |
| 05-31 | 36.45 | 36.27 | 0.18 | 68 | 67 | 1 | 100 | 70 | 100 | 70 |
| | 36.46 | 36.29 | 0.17 | 70 | 67 | 3 | 100 | 74 | 104 | 72 |
| | 36.13 | 36.37 | −0.2 | 71 | 72 | −1 | 100 | 72 | 102 | 70 |
| | 36.45 | 36.43 | 0.02 | 65 | 77 | −12 | 98 | 76 | 98 | 68 |
| | 36.48 | 36.76 | −0.3 | | | 0 | | | | |
| | 36.57 | 36.6 | −0 | 64 | 70 | −6 | 100 | 70 | 97 | 68 |
| 05-31 | 35.8 | 35.96 | −0.2 | | | 0 | | | | |
| 06-01 | 35.78 | 35.81 | −0 | 58 | 62 | −4 | 100 | 60 | 90 | 60 |
| | 36.74 | 36,72 | 0.02 | 67 | 58 | 9 | 120 | 80 | 105 | 70 |
| | 36.49 | 36.59 | −0.1 | | | 0 | | | | |
| | 35.78 | 35.81 | −0 | 60 | 64 | −4 | 110 | 68 | 110 | 60 |
| | | | | 61 | 65 | −4 | | | | |
| | | | 0 | | | 0 | | | | |
| 06-02 | 35.72 | 35.49 | 0.23 | 73 | 64 | 9 | 105 | 80 | 110 | 75 |
| | 36.31 | 36.43 | −0.1 | | | 0 | | | | |
| | 36.56 | 36.37 | 0.19 | | | 0 | | | | |
| | 36.37 | 36.18 | 0.19 | | | 0 | | | | |
| | 36.57 | 36.6 | −0 | | | 0 | | | | |
| 06-03 | 35.89 | 35.94 | −0.1 | | | 0 | | | | |
| | 36.26 | 36.46 | −0.2 | 60 | 63 | −3 | 110 | 80 | 105 | 70 |
| 06-04 | 35.65 | 35.82 | −0.2 | 64 | 62 | 2 | 100 | 70 | 104 | 70 |
| | 35.64 | 35.99 | −0.4 | 79 | 78 | 1 | 115 | 70 | 105 | 65 |
| | 36.47 | 36.26 | 0.21 | 72 | 64 | 8 | 110 | 78 | 100 | 70 |
| | | | 0 | 73 | 65 | 8 | 110 | 80 | 105 | 70 |
| | 36.56 | 36.52 | 0.04 | | | 0 | | | | |
| 06-05 | 36.03 | 36.12 | −0.1 | 65 | 60 | 5 | 110 | 70 | 100 | 65 |
| | 36.33 | 36.28 | 0.05 | | | 0 | | | | |
| 06-06 | 36.12 | 36.17 | −0.1 | | | 0 | 110 | 80 | 100 | 80 |
| | | | 0 | | | 0 | 106 | 78 | 116 | 88 |
| 06-07 | 35.87 | 36.09 | −0.2 | | | 0 | | | | |
| | 36.44 | 36.61 | −0.2 | | | 0 | | | | |
| 06-08 | 35.81 | 35.91 | −0.1 | | | 0 | | | | |
| | 36.51 | 36.71 | −0.2 | | | 0 | | | | |
| | 36.56 | 36.86 | −0.3 | | | 0 | | | | |
| | 36.75 | 36.87 | −0.1 | | | 0 | | | | |
| | 36.49 | 36.59 | −0.1 | | | 0 | | | | |
| 06-09 | 35.66 | 35.87 | −0.2 | | | 0 | 100 | 60 | 100 | 55 |
| | 36.31 | 36.14 | 0.17 | | | 0 | | | | |
| | 36.66 | 36.85 | −0.2 | | | 0 | 114 | 72 | 102 | 60 |
| 06-10 | 35.69 | 35.89 | −0.2 | | | 0 | 120 | 80 | 115 | 80 |
| | 36.59 | 36.69 | −0.1 | 70 | 68 | 2 | 110 | 75 | 108 | 70 |
| | 36.14 | 36.16 | −0 | 61 | 65 | −4 | 110 | 78 | 90 | 78 |
| 06-11 | 36.09 | 36.01 | 0.08 | 70 | 65 | 5 | 110 | 70 | 100 | 70 |
| | 36.82 | 36.82 | 0 | 74 | 64 | 10 | 110 | 60 | 110 | 65 |
| | | | | 69 | 65 | 4 | 109 | 73.81 | 104 | 70.7 |

For diabetes of heat physique characterized in heat in the upper and cold in the lower, treatment principles are just opposite to aforesaid patient. In an embodiment, an elderly female patient (born in 1942, height: 1.60 m, body weight: 71 kg) has a family history of diabetes. She has suffered from high blood glucose for 16 years. On May 17, 2009 she came to seek my medical advice. assay results on the day are stated as follows: glucose157, AIC7.5%, Tch6.01 mmol and TG2.5 mmol. 10-day average symmetrical life information difference-temperature difference during the period from May 17, 2009 to May 26, 2009 was 0.01. Thus, the cold physique of heat in the upper and cold in the lower was confirmed. Therefore, I implemented treatment to the patient with aforesaid methods, and adjusted her sleep orientation—head oriented towards the southeast with adequate sunshine and feet oriented towards the northwest that is well ventilated. As measured then, temperature difference between south and north walls was up to 1° C. From May 28, I requested the patient to pull pulley, life both hands and shake hands for 2 hours. Meanwhile, she was asked to immerse both feet into the foot bucket containing 42° C. hot water added with Chinese traditional herbal medicines while drinking 0° C. chilled water to maintain thermodynamic equilibrium. This lasted for 10 days, commencing from May 5. During this period, the patient stopped take drugs for oral administration and Insulin per day. From the 12th day, it was changed to injection before breakfast. Despite of this, the patient witnessed a reduction in blood glucose, from 157 at the beginning to 127 on May 2. Viewing from the following table, it can be seen that following reversed temperature regulation for 1-3 hours per time and pulley pull as well as lifting and shaking of hands for 1-3 hours per day, temperature difference was increasing towards negative difference. (See Table 17)

TABLE 17
Diabetes of Heat physique: Treatment to Syndrome of Heating in the Upper
and Cold in the Lower

| | Body | | | | | | | | | | | |
|---|---|---|---|---|---|---|---|---|---|---|---|---|
| | temperature ° C. | | | Pulse | | | BP | | | BP | | |
| | L | R | Temperature | (time/minute) | | | L | R | | L | | |
| Date | armpit | armpit | difference | L | R | | arm | arm | Difference | arm | R arm | Difference |
| 05-17 | 36.1 | 36.3 | −0.2 | | | | | | | | 70 | |
| 05-18 | 36.3 | 36.5 | −0.2 | | | | | | | | | |
| 05-19 | 36.1 | 36 | 0.1 | | | | | | | | | |
| 05-20 | 36.1 | 36 | 0.1 | | | | | | | | | |
| 05-21 | 36.1 | 36.2 | −0.1 | | | | | | | | | |
| 05-22 | 36 | 36 | 0 | | | | | | | | | |
| 05-23 | 36.2 | 36.1 | 0.1 | | | | | | | | | |
| 05-24 | 36.1 | 36 | 0.1 | | | | | | | | | |
| 05-25 | 36.1 | 36 | 0.1 | | | | | | | | | |
| 05-26 | 36.3 | 36.2 | 0.1 | | | | | | | | | |
| 05-27 | 36.1 | 36.2 | −0.1 | | | | | | | | | |
| 05-28 | 34.98 | 35.59 | −0.61 | | | 0 | | | 0 | | | 0 |
| 05-29 | 35.33 | 35.75 | −0.42 | 68 | 65 | 3 | 150 | 85 | 65 | 155 | 85 | 70 |
| 05-30 | 35.16 | 36.44 | −1.28 | | | 0 | | | 0 | | | 0 |
| 05-31 | 35.04 | 35.48 | −0.44 | | | 0 | | | 0 | | | 0 |
| 06-01 | 36.14 | 36.15 | −0.01 | 67 | 70 | −3 | 140 | 70 | 70 | 140 | 80 | 60 |
| 06-02 | 35.85 | 36.61 | −0.76 | 63 | 68 | −5 | 160 | 85 | 75 | 150 | 80 | 70 |
| 06-03 | 36.19 | 36.05 | 0.14 | 68 | 72 | −4 | 155 | 80 | 75 | 140 | 75 | 65 |
| 06-04 | 36.45 | 36.29 | 0.16 | 78 | 80 | −2 | 141 | 80 | 61 | 130 | 80 | 50 |
| 06-05 | 36.44 | 36.05 | 0.39 | 69 | 76 | −7 | 150 | 85 | 65 | 140 | 70 | 70 |
| | 35.768 | 36.06 | −0.13 | 69 | 72 | | 149.3 | 80.833 | 45.7 | 143 | 78.33 | 42.8 |

Integration of symmetrical body temperature and modern medical instruments will form a data correlation for precise definition of intervention orientation and quantity and improvement of curative effect without damage, pain and side effect.

### Example 25. Stress Echocardiography

Stress echocardiography (FIG. 14-15) was carried out at TIEN CARDIOLOGY ASSOCIATE, PC, New York on Aug. 10, 2020. At 08-1018:02:21.253, EKG was started (BP:L 145/90.70.R.161/108.94, 36.04-35.77° C., temperature difference: +0.27° C.). Firstly, the doctor placed 10 electrodes connected to ECG on the chest of the patient for pressure echocardiography (FIG. 16-20) in an attempt to check motions on the treadmill. Phase time: 00:25; speed: level: METS: 11; phase 33.4 MPH 14.0% HR: 142 BPM BP: level ST (J+60); ST gradient: QBS; amplitude: 1.1 mm 0.1 mv/sec0.7 mv 2.4 mv/s1.9 mv; recovery phase time: after 25-minute exercise, The temperature drop on the right side was significantly greater than that on the left side. Positive temperature difference increases gradually body temperature was 35.02-33.79° C.; temperature difference was +1.23° C.; R and L body temperature decreased by 1.98° C. and 1.02° C. respectively; 07:17; phase: 18:24; phase time: 07:17; speed: METS: HR: 0.MPH 92 BPM BP (42-minute exercise); body temperature was 36.24-34.90° C.; temperature difference was +1.34; R and L body temperature increased by 1.01° C. and 1.22° C. respectively; the treatment was terminated due to dyspnea and fatigue on the part of the patient

Diagnosis abstract: Duke treadmill score (DTS) is −11.6, indicating high risk. Good athletic ability is at Bruce Phase III. Exercise started at 6:30, and was terminated due to dyspnea accompanied by no chest pain on the part of patient. Datum line was HR70 BPM with maximum HR153 BPM up to 102% THR (Target Heart Rate) and METS 10 (Energy consumed during running, a unit indicating how many times the intensity of physical activity is equivalent to that at static status). Ischemic STT segment depressed without VTNF impression: ETT (Exercise Tolerance Test) when work load is satisfactory. (according to cardiology study, it was positive—positive indicator for conventional ETT lies in ST segment depression). The stress echo report is to be submitted separately. These data prove that when the temperature difference increases by more than 0.6° C., the ST segment is depressed, and clinical symptoms such as palpitation, chest pain, and dyspnea appear, the possibility of myocardial ischemia must be considered.

When dyspnea and fatigue were found, the left and right body temperatures rose above 1° C. in one hour; temperature difference was over 1° C. If left body temperature is high, it means the physique of heat in the upper and cold in the lower; it is applicable to heat the lower body, and vice versa. Thus, treatment orientation can be defined.

MetS is a unit indicating how many times the intensity of physical activity is equivalent to that at static status. It is 1 mets in case of sitting in quiet. As indicated by pressure echocardiography for examination of motions on the treadmill, right body temperature gradually decreased while temperature difference and Mets were gradually increasing. When exercise was carried out for 22 minutes, body temperature was 35.02-33.79° C., and temperature difference was +1.23° C.; R and L body temperature decreased by 1.98° C. and 1.02° C. respectively. METS: 11; we can see clearly the correlation between Mets and decrease of right-left body temperature and increase of temperature difference. When implementing remote monitoring of the intensity of physical activities, the impact on health is favorable for monitoring the intensity of health activity.

Real-time monitoring of symmetrical body temperature (see Table 18) can serve as an objective of reference for datum line HR, maximum HR, THR and blood pressure as well as STT segment. Furthermore, it is also applicable to use the method of treating cold syndrome with hot natured drugs and vice versa) to regulate and control variation to heart rate, blood pressure and STT segment. Datum line HR 70 BPM in this case and maximum HR153 BPM are up to 102% THR; for variation to blood pressure and ST-T segment before and after exercise on the treadmill, please see attachments. We can see clearly the correlation between Mets and decrease of right-left body temperature and increase of temperature difference. When implementing remote monitoring of the intensity of physical activities, the impact on health is favorable for monitoring the intensity of health activity.

TABLE 18
Phase Time Based Data on Body Temperature and Temperature Difference

| | L | R | Temper- |
|---|---|---|---|
| | Temper- | Temper- | ature |
| Date/time | ature | ature | difference |
| 08-10 19:52:29 | 36.21 | 36.08 | +0.13 |
| 08-10 19:51:27 | 36.19 | 36.03 | +0.16 |
| 08-10 19:50:23 | 36.16 | 35.96 | +0.20 |
| 08-10 19:49:20 | 36.14 | 35.99 | +0.15 |
| 08-10 19:48:18 | 36.11 | 35.96 | +0.15 |
| 08-10 19:47:15 | 36.10 | 35.90 | +0.20 |
| 08-10 19:46:11 | 36.07 | 35.81 | +0.26 |
| 08-10 19:45:07 | 36.04 | 35.78 | +0.26 |
| 08-10 19:44:05 | 36.03 | 35.70 | +0.33 |
| 08-10 19:43:03 | 36.06 | 35.71 | +0.35 |
| 08-10 19:41:59 | 36.08 | 35.67 | +0.41 |
| 08-10 19:40:56 | 36.14 | 35.58 | +0.56 |
| 08-10 19:39:53 | 36.32 | 35.53 | +0.79 |
| 08-10 19:38:50 | 36.27 | 35.28 | +0.99 |
| 08-10 19:37:48 | 36.22 | 35.26 | +0.96 |
| 08-10 19:36:44 | 36.17 | 35.19 | +0.98 |
| 08-10 19:35:41 | 36.11 | 35.07 | +1.04 |
| 08-10 19:34:39 | 36.04 | 34.90 | +1.14 |
| 08-10 19:33:36 | 35.97 | 34.77 | +1.20 |
| 08-10 19:32:32 | 35.90 | 34.58 | +1.32 |
| 08-10 19:31:30 | 35.87 | 34.48 | +1.39 |
| 08-10 19:30:27 | 35.94 | 34.74 | +1.20 |
| 08-10 19:29:24 | 36.00 | 34.89 | +1.11 |
| 08-10 19:28:21 | 36.01 | 34.81 | +1.20 |
| 08-10 19:27:18 | 36.04 | 34.84 | +1.20 |
| 08-10 19:26:15 | 36.16 | 34.73 | +1.43 |
| 08-10 19:25:13 | 36.15 | 34.74 | +1.41 |
| 08-10 19:24:09 | 36.15 | 34.84 | +1.31 |
| 08-10 19:23:06 | 36.20 | 35.06 | +1.14 |
| 08-10 19:22:04 | 36.17 | 34.93 | +1.24 |
| 08-10 19:21:02 | 36.21 | 35.13 | +1.08 |
| 08-10 19:19:57 | 36.21 | 35.02 | +1.19 |
| 08-10 19:18:55 | 36.22 | 34.99 | +1.23 |
| 08-10 19:17:52 | 36.24 | 34.90 | +1.34 |

07:17 phase: phase time: 07:17; speed: level: METS:
HR: 0. MPH 92 BPM BP (42 minutes)

| | | | |
|---|---|---|---|
| 08-10 19:16:50 | 36.21 | 35.04 | +1.17 |
| 08-10 19:15:46 | 36.18 | 35.00 | +1.18 |
| 08-10 19:14:43 | 36.14 | 34.97 | +1.17 |
| 08-10 19:13:40 | 36.10 | 34.99 | +1.11 |
| 08-10 19:12:38 | 36.06 | 34.92 | +1.14 |
| 08-10 19:11:34 | 36.04 | 35.19 | +0.85 |
| 08-10 19:10:31 | 36.01 | 35.06 | +0.95 |
| 08-10 19:09:29 | 35.97 | 34.96 | +1.01 |
| 08-10 19:08:26 | 35.94 | 34.70 | +1.24 |
| 08-10 19:07:22 | 35.94 | 34.92 | +1.02 |
| 08-10 19:06:20 | 35.93 | 34.79 | +1.14 |
| 08-10 19:05:17 | 35.91 | 34.72 | +1.19 |
| 08-10 19:04:15 | 35.90 | 34.71 | +1.19 |
| 08-10 19:03:11 | 35.87 | 34.61 | +1.26 |
| 08-10 19:02:08 | 35.83 | 34.50 | +1.33 |
| 08-10 19:01:06 | 35.77 | 34.36 | +1.41 |
| 08-10 19:00:03 | 35.76 | 34.27 | +1.49 |
| 08-10 18:58:59 | 35.78 | 34.43 | +1.35 |
| 08-10 18:57:56 | 35.79 | 34.22 | +1.57 |
| 08-10 18:56:53 | 35.78 | 34.14 | +1.64 |
| 08-10 18:55:50 | 35.83 | 34.31 | +1.52 |
| 08-10 18:54:47 | 35.84 | 34.21 | +1.63 |
| 08-10 18:53:44 | 35.81 | 34.17 | +1.64 |
| 08-10 18:52:41 | 35.81 | 34.05 | +1.76 |
| 08-10 18:51:39 | 35.77 | 34.34 | +1.43 |
| 08-10 18:50:35 | 35.75 | 34.06 | +1.69 |
| 08-10 18:49:32 | 35.70 | 33.91 | +1.79 |
| 08-10 18:48:30 | 35.67 | 33.97 | +1.70 |
| 08-10 18:47:27 | 35.64 | 33.78 | +1.86 |
| 08-10 18:46:23 | 35.64 | 33.97 | +1.67 |
| 08-10 18:45:20 | 35.71 | 33.92 | +1.79 |
| 08-10 18:44:17 | 35.75 | 33.88 | +1.87 |
| 08-10 18:43:14 | 35.78 | 33.93 | +1.85 |
| 08-10 18:42:10 | 35.79 | 33.88 | +1.91 |
| 08-10 18:41:08 | 35.79 | 34.20 | +1.59 |
| 08-10 18:40:05 | 35.74 | 34.33 | +1.41 |
| 08-10 18:39:03 | 35.74 | 34.32 | +1.42 |
| 08-10 18:37:59 | 35.81 | 34.39 | +1.42 |
| 08-10 18:36:56 | 35.85 | 34.66 | +1.19 |
| 08-10 18:35:54 | 35.78 | 34.78 | +1.00 |
| 08-10 18:34:51 | 35.69 | 34.66 | +1.03 |
| 08-10 18:33:47 | 35.60 | 34.45 | +1.15 |
| 08-10 18:32:45 | 35.51 | 34.35 | +1.16 |
| 08-10 18:31:42 | 35.42 | 34.25 | +1.17 |
| 08-10 18:30:40 | 35.34 | 34.18 | +1.16 |
| 08-10 18:29:36 | 35.27 | 34.24 | +1.03 |
| 08-10 18:28:33 | 35.17 | 33.91 | +1.26 |
| 08-10 18:27:31 | 35.15 | 34.21 | +0.94 |
| 08-10 18:26:28 | 35.00 | 34.02 | +0.98 |
| 08-10 18:25:24 | 35.02 | 33.79 | +1.23 |

06:25 phase: phase time: 00:25; speed: level: METS: 11; phase: 33.4
MPH14.0% HR: 142BPM BP: ST (J + 60)ST gradient

| | | | |
|---|---|---|---|
| 08-10 18:24:22 | 34.91 | 33.77 | +1.14 |

QRSamplitude-1.1 mm 0.1 mv/sec 0.7 mv 2.4 mv/s
1.9 mv, recovery phase time:

| | | | |
|---|---|---|---|
| 08-10 18:23:19 | 34.80 | 33.77 | +1.03 |
| 08-10 18:22:16 | 34.68 | 33.60 | +1.08 |
| 08-10 18:21:13 | 34.50 | 33.92 | +0.58 |
| 08-10 18:20:10 | 34.35 | 34.09 | +0.26 |
| 08-10 18:19:07 | 34.36 | 33.79 | +0.57 |
| 08-10 18:18:04 | 34.50 | 33.52 | +0.98 |
| 08-10 18:17:01 | 34.36 | 33.00 | +1.36 |
| 08-10 18:15:59 | 34.33 | 33.12 | +1.21 |
| 08-10 18:14:56 | 34.50 | 33.46 | +1.04 |
| 08-10 18:13:52 | 34.74 | 33.91 | +0.83 |
| 08-10 18:12:50 | 34.99 | 33.67 | +1.32 |
| 08-10 18:11:47 | 35.17 | 33.85 | +1.32 |
| 08-10 18:10:44 | 35.57 | 35.03 | +0.54 |
| 08-10 18:09:41 | 35.85 | 35.74 | +0.11 |
| 08-10 18:08:38 | 36.02 | 35.68 | +0.34 |
| 08-10 18:07:35 | 36.08 | 35.58 | +0.50 |
| 08-10 18:06:33 | 36.08 | 35.45 | +0.63 |
| 08-10 18:05:29 | 36.06 | 35.39 | +0.67 |
| 08-10 18:04:26 | 36.05 | 35.44 | +0.61 |
| 08-10 18:03:24 | 36.05 | 35.60 | +0.45 |
| 08-10 18:02:21 | 36.04 | 35.77 | +0.27 |

08-10 18:02:21, start EKG.BP.145/90.70.161/108.94

Such sports related physiological instruments and equipment as COSMED Quark PFT ergo cardiopulmonary function tester, COSMED K5 telemetry metabolism tester and intelligent muscle oxygen monitor can provide such symmetrical physiological data as right-left body temperature, pulse, blood pressure, blood oxygen and blood glucose as well as correlation between increase and decrease of their differences before and after sports. When implementing remote monitoring of the intensity of physical activities, the impact on health is favorable for monitoring the intensity of health activity.

Such cold-heat testing instruments as infrared thermal imager for medical purpose can provide distribution of thermal field at lesion site, infer its circulation and metabolic state, and judge the nature and intensity of the lesion and the area affected on this basis. Human body is a biological heating mass. As anatomical structure, tissue metabolism, blood circulation and nerve state are varied, different temperature at various body parts will form different heat fields. As to assessment of recovery of physiological functions and blood circulation, it can realize positioning to a specific organ for assessment. However, precision of non-contact temperature measurement at present is below ±0.2 due to difficulties with functional measurement. While implementing tests with the help of infrared thermal imager, we may proceed with real-time measurement of user's physiological data, such as symmetrical body temperature, pulse, blood pressure, blood oxygen and blood glucose. This aims to establish a correlation in terms of data, which can improve diagnosis precision of infrared thermal imager.

### Example 26. Stipulate More Scientific Criteria of Screening, Examination and Discharge for COVID-19

In an embodiment, the symmetrical equilibrium IOT in individualized remote health management system for dynamic observation according to the present invention aims to stipulate more scientific criteria for screening, examination and discharge of COVID-19 population; it is also expected to proceed with remote health management of sequela in an attempt to reduce recurrence rate, improve overall quality and production efficiency. Symmetrical temperature measurement is an effective method for “early discovery”

COVID-19 is a global infectious disease. To focus on epidemics, it is essential to pay attention to the source of infection. “Early discovery and isolation are more important than anything”, especially any person without such syndromes as fever, namely Asymptomatic Carrier. Measurement of body temperature is the most sensitive and common approach for identification of Asymptomatic Carriers. As indicated by The Lancet, 83% COVID-19 have the syndrome of fever while remaining 17% patients have no fever at all. This is an important reason for missed diagnosis of potential virus carriers. Minimization of missed diagnosis of such asymptomatic patient is an important measure for minimization of mobile source of infection. Our discoveries obtained through 12-year study of symmetrical body temperature: Two-side symmetrical body temperature difference of a person in good health is below 0.2° C.; however, such difference is to be up to 0.3-1.8° C. in the state of disease; taking temperature measurement for instance, one side of lower temperature has no fever, which is contrary to the other side; if two-side body temperature difference is over 0.3° C., it means that the body is imbalanced. This is a positive indicator indicating a state of recessive disease. It is also an important reason for missed diagnosis.

Precautions for positive temperature measurement and handling: 1) body temperature is abnormal: for patients to whom no treatment measures are taken, it is still necessary to proceed with observation if temperature on one side is over 37.3° C. or two-side temperature difference is over 0.3° C., even though it incurred by other causes. Selection of items related to patients will not take too much time, which can minimize missed diagnosis of source of infection. 2) It is essential to proceed with measurement before any treatment measure is taken; body temperature is an extremely sensitive indicator, which may result in variation to temperature difference. 3) Basal body temperature after wake-up in the morning is relatively reliable, which can be used to define the physique of a patient. Therefore, it is essential to measure basal body temperature every day. 4) gradual reduction in basal body temperature difference on each day is a reflection of good turn to state of disease, and vice versa. 5) Symmetrical temperature measurement after three meals and before sleep serves as an observation of dynamic functions of suspected patients, which can improve positive detection rate. 6) Syndromes and selection of epidemiological history table: If there is any history of travel or residence in epidemics area, if there is any personal contact with infected persons, if there is an epidemiological link with COVID-19 infection, and if there is a cough. Where any of such items is identified, corresponding patient is to be deemed as the patient under observation, who is requested to subject to the following “isolated examination”

### Example 27. Design of Symmetrical Thermal Imaging Monitor

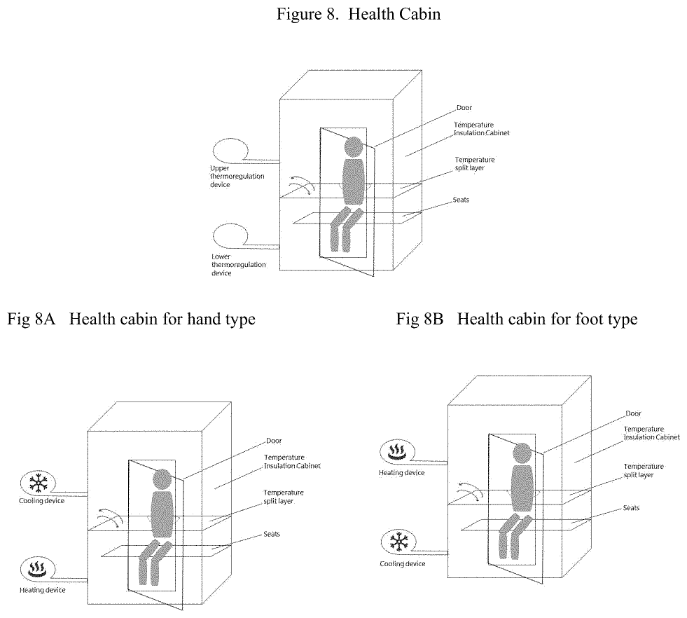

We designed a “symmetrical infrared body temperature rapid screening instrument”, which can simultaneously measure the symmetrical body temperatures of temples, ear sockets and wrists on both sides. Body heat was determined by symmetrical body temperatures and temperature difference. Compared with the traditional one-sided measurement, it can reduce the number of missed fever patients and reduce the spread of infectious sources. The method of manually holding a non-contact infrared forehead thermometer is as follows: first measure the body temperature of the left ear fossa or temple (the two signs are symmetrical), and then measure the right body temperature. When the temperature on one side exceeds 37.3° C. and the temperature difference on both sides exceeds 0.3° C., positive shall be considered (see FIG. 8) 1) In April 2020, after consulting the data from Wuhan, we monitored 10 patients with infrared imaging (these 10 COVID-19 patients came from the shelter hospital, had no fever, negative in nucleic acid examination, and were transferred to the regional hospital for isolation). The total number of test times was 20. Each time, the tested positions were left/right temples, left/right Taiyuan acupoint, both sides of forehead and bilateral radial artery pulsation parts. After that, we found that the bilateral temperature difference of the patients was 100%, and at least one side was between ±0.35-1° C. and 1.5° C. These contents are the objective basis for isolation, indicating that the temperature difference is more accurate than the body temperature. The temperature difference of 0.35-1.5° C. in different parts of forehead/wrists is related to the patient's physique and other medical diseases. It is a more sensitive biomarker indicating physiological and pathological conditions than unilateral body temperature. These data show that after discharge, the patients bodies had not fully recovered its balance, but through these data, mild and some asymptomatic patients can be found as soon as possible. When any patient has no fever but temperature difference, it is necessary to continue to observe the patient in order to reduce the source of infection. The calculation results of one embodiment are shown in Table 19.

TABLE 19
Temperature Measurement and Calculations

| Patient | Patient | | L | R | | | L | R | |
|---|---|---|---|---|---|---|---|---|---|
| Number | Age | Gender | temple | temple | Difference | Ophryon | Taiyuan | Taiyuan | Difference |
| 1 | 30 | Male | 34.22 | 34.12 | −0.1 | 33.82 | 33.22 | 34.22 | −1 |
| 1 | 30 | Male | 32.92 | 31.4 | −1.52 | 33.22 | 31.4 | 32.11 | −0.71 |
| 2 | 40 | Male | 33.32 | 33.42 | 0.1 | 33.62 | 28.83 | 29.14 | −0.31 |
| 2 | 40 | Male | 33.42 | 34.41 | 0.99 | 33.92 | 30.89 | 31.1 | −0.21 |
| 3 | 48 | Male | 32.21 | 32.01 | −0.2 | 32.11 | 31.2 | 31.3 | −0.1 |
| 3 | 48 | Male | 31.1 | 31.3 | 0.2 | 30.89 | 31.3 | 30.79 | 0.51 |
| 4 | 51 | Male | 33.12 | 32.82 | −0.3 | 33.12 | 31.91 | 32.01 | −0.1 |
| 4 | 51 | Male | 31.5 | 31.81 | 0.31 | 32.21 | 30.99 | 29.66 | 1.33 |
| 5 | 49 | Female | 29.86 | 28.93 | −0.93 | 29.55 | 29.45 | 29.04 | 0.41 |
| 5 | 49 | Female | 31.61 | 31.5 | −0.11 | 32.52 | 31.81 | 32.42 | −0.61 |
| 5 | 49 | Female | 32.31 | 30.79 | −1.52 | 31.91 | 29.04 | 29.14 | −0.1 |
| 6 | 51 | Female | 31.61 | 33.42 | 1.81 | 33.32 | 32.82 | 33.12 | −0.3 |
| 7 | 55 | Female | 33.92 | 34.22 | 0.3 | 33.32 | 33.32 | 33.82 | −0.5 |
| 7 | 55 | Female | 29.45 | 29.55 | 0.1 | 29.55 | 30.69 | 31.5 | −0.81 |
| 8 | 60 | Female | 31.1 | 32.62 | 1.52 | 31.1 | 31.81 | 31.1 | 0.71 |
| 8 | 60 | Female | 31.91 | 32.31 | 0.4 | 32.92 | 31.2 | 29.86 | 1.34 |
| 9 | 65 | Female | 34.61 | 34.22 | −0.39 | 33.32 | 34.12 | 34.12 | 0 |
| 9 | 65 | Female | 30.89 | 30.38 | −0.51 | 32.82 | 30.99 | 32.01 | −1.02 |
| 10 | 73 | Female | 29.66 | 31.1 | 1.44 | 30.17 | 29.55 | 29.35 | 0.2 |
| 10 | 73 | Female | 27.99 | 29.97 | 1.98 | 30.07 | 28.2 | 26.83 | 1.37 |
| 10 | 73 | Female | 34.41 | 34.02 | −0.39 | 31.2 | 27.89 | 28.31 | −0.42 |

The monitoring design of symmetrical thermal imager is as follows: 1. intelligent temperature measurement algorithm: when screening body temperatures, it is necessary to determine a body part with index significance. This part shall provide reliable data in the symmetrical parts of the person, and then the machine learning technology is used to automatically and accurately find the temperature collection areas of two symmetrical parts such as temples and ears from the thermal imaging side view, so as to calculate the temperature of each side; 2. connect the infrared thermometer with the computer monitoring system, so as to display the left and right measured temperatures on the computer display screen, and calculate the symmetrical temperature difference. When the temperature difference between two sides is more than 0.3° C., or the temperature on one side is higher than 37.3° C., the system will give an early warning; and 3. focus on prevention and prevent disease before it happens: use the preventive symmetrical temperature measurement APP to help people measure the temperature symmetrically, especially the basic body temperature. Basic body temperature measurement must be taken for isolated persons with normal body temperature for 10 consecutive days. When any body temperature exceeds 37.3° C. and the temperature difference exceeds 0.3° C., it shall be considered positive and the patient shall be subject to special examination. The treatment direction can be determined by monitoring the difference of symmetrical body surface life information every day and before and after treatment. When the balance coefficient of life information on the symmetrical body surfaces is reduced or reverse conversion occurs, it indicates that the method is effective or the patient is improved. By combining the qualitative, semi-quantitative and quantitative methods with the above monitoring contents, and expressing the results in numbers, a large database of COVID-19 can be developed. At the same time, the application of statistics and AI to monitor physique can determine the impact of curative effect, rehabilitation direction, health status and life style on it.

What is claimed is:

1. A method for individualized remote health management through dynamic observation of variations to an individual patient's physiological indicators, comprising the steps of: (i) measuring said physiological indicators from symmetric positions on the right and left sides of said individual patient at the same time point; (ii) sending data on said individual patient's physiological indicators for synchronous AI real-time processing; (iii) comparing said data in step (ii) with reference to perfectly healthy individuals; (iv) engaging in real-time targeted health intervention when the data from Step (ii) deviate from that of the reference, wherein said physiological indicators comprise body temperature, pulse, blood pressure, blood oxygen and blood glucose.

2. The method of claim 1, wherein said physiological indicator is symmetrical basic body temperature, wherein said symmetrical basic body temperature is measured by a data acquisition device for 10 days before making a decision on health intervention.

3. The method of claim 2, wherein said data acquisition device is (i) a mechanical or (ii) electronic temperature sensor with symmetrical left and right contact with the human body to accurately observe and record the human body temperature under stable conditions, or (iii) a non-contact symmetrical left and right infrared thermometer to quickly screen said individual patient with unbalanced body temperature.

4. The system according to claim 1, characterized in that the said the data acquisition device is a syndrome input device to detect acute and subacute attacks of chronic diseases which may be accompanied by said syndromes by detecting dynamic variation to left and right body temperature, pulse, blood pressure, blood oxygen and/or blood glucose.

5. The method of claim 1, characterized in establishing a standard for establishing human health status by using symmetrical life information on human body surface, wherein healthy people has a temperature difference of less than 0.2° C., the same blood pressures, and the same pulse; wherein sub-healthy people has a temperature difference of more than 0.2° C., and the pulse difference is 1; wherein people in disease stage has a temperature difference of 0.3-0.35° C., and the pulse difference of 2; and wherein severely ill patient has a temperature difference of more than 0.45° C. or the pulse difference of more than 3.

6. The method of claim 5, characterized in that the data acquisition device automatically determines whether said physiological indicators of said individual patient fall into the range for severely ill patient.

7. The method of claim 2, wherein said data acquisition device, together with a smartphone APP, can take food photos and upload them to the server for calculation of calories and analyzation of food types through AI, and suggest whether the food is suitable for said individual patient according to the food's properties, and the individual patient's personal profile.

8. The method of claim 1, wherein said data acquisition device comprises a synchronous environment and personal environment information acquisition device which is used to measure the environment and personal environment information of the user's location while the symmetrical balance acquisition device measures the physiological data, and to send the information through the Internet to a data processing platform; wherein said data processing platform quantifies the relationship between the local environment and personal environment, and dynamically expresses the symmetrical balance of the relationship between the dynamic symmetrical physiological data difference of health impact and the physiological state information corresponding to environmental information.

9. The method of claim 2, wherein said data acquisition device combines symmetrical balanced physique, dynamic data, modern medical instrument monitoring and monitoring movement, so as to find the direction of targeted health intervention.

10. The method of claim 1, characterized in that according to the changes of body temperature in symmetrical balanced physique, dynamic data and rehabilitation, physical therapy, targeted therapy and monitoring, said data acquisition device finds the direction of targeted rehabilitation according to temperature difference, and determines the prescription of targeted intervention with artificial intelligence, so as to integrate information acquisition, testing, diagnosis and intervention.

11. The method of claim 2, characterized by screening, checking and formulating scientific discharging standards for COVID-19 patients through the infrared thermometer, and carrying out remote health management for sequelae so as to reduce recurrence rate, alleviate sequelae, improve overall quality and increase production efficiency.

12. The method of claim 1, characterized in determining the individualized advantages and disadvantages and degree to health by calculating the balance coefficient and comparing the balance coefficient before and after a specific behavior, so as to realize individualized health management and form a health intervention method for dynamically observing physiological data.

13. The method of claim 12, characterized in that the balance coefficient refers to the direction and value of the difference between dynamic symmetrical physiological data, and the direction and value of the increase or decrease of the information difference are the objective standard for health intervention, wherein the symptoms of chronic diseases, acute diseases and sub-acute diseases, take 1 to 10 as the reference for degree, from 0 for feeling good to 10 for unbearable with the middle of 5, wherein these data values correspond to symmetrical life information and observe the quantitative relationship between physiological data, physiological data difference and symptom intensity.

14. The method of claim 8, characterized in that the diagnosis method includes using heavenly stem data, wherein ten days constitute a heavenly stem (1-10-20-30 of the lunar calendar) to represent internal organs, wherein said method is suitable for accurately determining human physique according to 10 day basic body temperature and specific mathematical model.

15. The method of claim 12, wherein the behaviors include the specific behaviors such as lifestyle, environmental factors, psychological activities, and intervention means; wherein the increased balance coefficient after behavior indicates that the behavior is not suitable for the patients and shall be stopped and the decreased balance coefficient indicates that the behavior is beneficial to the patients and can continue.

16. The method of claim 12 wherein t the individualized health management includes using the data provided by the symmetrical life information, the direction and value of the increase or decrease of the physiological data difference before and after the health intervention are the objective standards for the health intervention, and the dynamic evaluation is carried out by using the signs, physique, lifestyle and environmental data; wherein an increased balance coefficient after intervention indicates that the behavior is not suitable for the patients and shall be stopped and a decreased balance coefficient indicates that the behavior is beneficial to the patients and can continue.

17. The method of claim 12, wherein the diagnosis and treatment system measures symmetrical life information before and after different health intervention methods to find the best individualized health intervention measures according to the differences and physical coupling degree.

18. The method of claim 2, wherein a wireless symmetrical thermometer for body temperature is used; wherein artificial intelligence calculation, the thermometer overcomes the temperature change caused by movement and turning over of symmetrical body temperature of armpits; wherein, the accuracy of collecting symmetrical body temperature is ensured through the fixing and thermal insulation functions of the wearable device; wherein gyroscopes and acceleration sensors are used to determine the start and end time of lifestyle as well as movement direction and state, so as to ensure the accuracy of individualized testing.

19. The method of claim 1, wherein intelligent health balance cabin is used; wherein according to the treatment needs, said intelligent health balance cabin is divided into the following types: type A is used for patients with upper cold and lower hot, and type B is used for patients with upper hot and lower cold; wherein the functions are as follows: a. the upper and lower temperature areas are used to adjust the temperature independently according to the prescription; b. humidity; c. wind force and wind direction (air conditioning), d. concentration and proportion of air components; e. sound; f. vision; g. the amount of trace elements, natural animal and plant products, chemicals and other products in contact with human body; h. contact tightness, pressure and quantity of the above items.

20. The method of claim 19, further comprising the use of the internal environment and emotions such as cold, heat, water, electrolyte and interstitial pressure of the human body are affected and adjusted, and the surrounding environment is changed so that the patient does not contact with adverse stimulating factors, but only with the external environment conducive to the body, so as to control different items.
